SEQUENCE LISTING (104 sequences)

SEQ ID NO: 1 - protein, 116 aa, Artificial sequence
  synthesized
  misc_feature: mAb035 VH
QVQLQQSGAELVRPGASVTLSCKASDYTFTDYEMHWVYQTPVHGLEWIGAIDPETGGTAYNQKFKDKAILTADKSSNTAYMELRSLTSEDSAVYFCTKTGYQFDYWGQGTTLTVSS

SEQ ID NO: 2 - DNA, 348 bp, Artificial sequence
  synthesized
  misc_feature: mAb035 VH
caggttcaactgcagcagtctggggctgagctggtgaggcctggggcttcagtgacgctgtcctgcaaggcttcggactacacatttactgactatgaaatgcactgggtgtaccagacacctgtacatggcctggaatggattggagctattgatcctgaaactggtggtactgcctacaatcagaagttcaaggacaaggccatactgactgcagacaaatcctccaacacagcctacatggagctccgcagcctgacatctgaggactctgccgtctatttctgtacaaaaactgggtaccagtttgactactggggccaaggcaccactctcacagtctcctca

SEQ ID NO: 3 - protein, 5 aa, Artificial sequence
  synthesized
  misc_feature: mAb035 VH-CDR1
DYEMH

SEQ ID NO: 4 - protein, 17 aa, Artificial sequence
  synthesized
  misc_feature: mAb035 VH-CDR2
AIDPETGGTAYNQKFKD

SEQ ID NO: 5 - protein, 7 aa, Artificial sequence
  synthesized
  misc_feature: mAb035 VH-CDR3
TGYQFDY

SEQ ID NO: 6 - protein, 107 aa, Artificial sequence
  synthesized
  misc_feature: mAb035 VL
DIVMTQSQKFMSTSVGDRVSVTCKASQNVGSNVAWYQQKPGQSPKALIYSASYRYSGVPDRFAGSGSGTDFTLTISNVQSEDLADYFCQQYNSHPYTFGGGTKLEIK

SEQ ID NO: 7 - DNA, 321 bp, Artificial sequence
  synthesized
  misc_feature: mAb035 VL
gacattgtgatgacccagtctcaaaaattcatgtccacatcagtaggagacagggtcagcgtcacctgcaaggccagtcagaatgtgggtagtaatgtagcctggtatcaacagaaaccaggacaatctcctaaagcactgatttattcggcatcctaccggtacagtggagtccctgatcgcttcgcaggcagtggatctgggacagatttcactctcaccatcagcaatgtgcagtctgaagacctggcagattatttctgtcagcaatacaacagccatccgtacacgttcggaggggggaccaagctggaaataaaa

SEQ ID NO: 8 - protein, 11 aa, Artificial sequence
  synthesized
  misc_feature: mAb035 VL-CDR1
KASQNVGSNVA

SEQ ID NO: 9 - protein, 7 aa, Artificial sequence
  synthesized
  misc_feature: mAb035 VL-CDR2
SASYRYS

SEQ ID NO: 10 - protein, 9 aa, Artificial sequence
  synthesized
  misc_feature: mAb035 VL-CDR3
QQYNSHPYT

SEQ ID NO: 11 - protein, 112 aa, Artificial sequence
  synthesized
  misc_feature: mAb053 VH
QVQLKQSGPGLVQPSQSLSIICTVSGFSLSDYGVMWVRQSPGKGLEWLGVIWSGGSTDYNAAFTSRLSISRDNSKRQVIFKMNSLQAEDTAIHYCVRETMDYWGQGTSVTVS

SEQ ID NO: 12 - DNA, 339 bp, Artificial sequence
  synthesized
  misc_feature: mAb053 VH
caggtgcagctgaagcagtcaggacctggcctagtgcagccctcacagagcctgtccatcatatgcacagtatctggtttctcattaagtgactatggtgtaatgtgggttcgccagtctccaggaaagggtctggagtggctgggagtgatatggagtggtggaagtacagactataatgcagctttcacatccagactgagcatcagcagggacaattccaagagacaagtcatctttaaaatgaacagtctacaagctgaggacacagccatacattactgtgtcagagaaacgatggactactggggtcaaggaacctcagtcaccgtctcctca

SEQ ID NO: 13 - protein, 5 aa, Artificial sequence
  synthesized
  misc_feature: mAb053 VH-CDR1
DYGVM

SEQ ID NO: 14 - protein, 16 aa, Artificial sequence
  synthesized
  misc_feature: mAb053 VH-CDR2
VIWSGGSTDYNAAFTS

SEQ ID NO: 15 - protein, 5 aa, Artificial sequence
  synthesized
  misc_feature: mAb053 VH-CDR3
ETMDY

SEQ ID NO: 16 - protein, 107 aa, Artificial sequence
  synthesized
  misc_feature: mAb053 VL
DIQMTQTTSSLSASLGDRVTISCRASQDISNYLNWYQQRPDGTVKRLIYYTSRLHSGVPSRFSGSGSGTDYSLTISSLEQEDIATYFCQQAHTLPWTFGGGTKLEIK

SEQ ID NO: 17 - DNA, 321 bp, Artificial sequence
  synthesized
  misc_feature: mAb053 VL
gatatccagatgacacagactacatcctccctgtctgcctctctgggagacagagtcaccatcagttgcagggcaagtcaggacattagcaattatttaaactggtatcagcagagaccagatggaactgttaaacgcctaatctactacacatcaagattacactcaggagtcccatcaaggttcagtggcagtgggtctggaacagattattctctcaccattagcagcctggagcaagaagacattgccacttacttttgccaacaggctcatacgcttccgtggacgttcggtggaggcaccaagttggagatcaaa

SEQ ID NO: 18 - protein, 11 aa, Artificial sequence
  synthesized
  misc_feature: mAb053 VL-CDR1
RASQDISNYLN

SEQ ID NO: 19 - protein, 7 aa, Artificial sequence
  synthesized
  misc_feature: mAb053 VL-CDR2
YTSRLHS

SEQ ID NO: 20 - protein, 9 aa, Artificial sequence
  synthesized
  misc_feature: mAb053 VL-CDR3
QQAHTLPWT

SEQ ID NO: 21 - protein, 112 aa, Artificial sequence
  synthesized
  misc_feature: mAb041 VH
EVQLVESGGGLVKPGGSLKLSCAVSGFTFSNSAMSWVRQTPEKRLEWVATITDGGDYTYYSDNVKGRFTISRDNAKNNLYLQMSHLKSEDTAMYYCVRGISYWGQGTLVTVS

SEQ ID NO: 22 - DNA, 339 bp, Artificial sequence
  synthesized
  misc_feature: mAb041 VH
gaagtgcagctggtggagtctgggggaggcttagtgaagcctggagggtccctgaaactctcctgtgcagtctctggattcactttcagtaactctgccatgtcttgggttcgccagactccggaaaagaggctggagtgggtcgcaaccattactgatggtggtgattacacctactattcagacaatgtaaagggccgattcaccatctccagagacaatgccaagaacaacctgtacctgcaaatgagccatctgaagtctgaggacacagccatgtattactgtgtaagagggatttcttactggggccaagggactctggtcactgtctctgca

SEQ ID NO: 23 - protein, 5 aa, Artificial sequence
  synthesized
  misc_feature: mAb041 VH-CDR1
NSAMS

SEQ ID NO: 24 - protein, 17 aa, Artificial sequence
  synthesized
  misc_feature: mAb041 VH-CDR2
TITDGGDYTYYSDNVKG

SEQ ID NO: 25 - protein, 4 aa, Artificial sequence
  synthesized
  misc_feature: mAb041 VH-CDR3
GISY

SEQ ID NO: 26 - protein, 111 aa, Artificial sequence
  synthesized
  misc_feature: mAb041 VL
DIVLTQSPASLAVSLGQRATISCRASKSVSTSSDSYMHWYQQKPGQPPKLLIKYASNLESGVPVRFSGSGSGTDFTLNIHPVEEEDAATYYCQHSREFPWTFGGGTKLEIK

SEQ ID NO: 27 - DNA, 333 bp, Artificial sequence
  synthesized
  misc_feature: mAb041 VL
gacattgtgctgacacagtctcctgcttccttagctgtatctctggggcagagggccaccatctcctgcagggccagcaaaagtgtcagtacatctagcgatagttacatgcactggtaccaacagaaaccaggacagccgcccaaactcctcatcaagtatgcatccaacctagaatctggggtccctgtcaggttcagtggcagtgggtctgggacagacttcaccctcaacatccatcctgtggaggaggaggatgctgcaacatattattgtcagcacagtagggagtttccgtggacgttcggtggaggcaccaagctggaaatcaaa

SEQ ID NO: 28 - protein, 15 aa, Artificial sequence
  synthesized
  misc_feature: mAb041 VL-CDR1
RASKSVSTSSDSYMH

SEQ ID NO: 29 - protein, 7 aa, Artificial sequence
  synthesized
  misc_feature: mAb041 VL-CDR2
YASNLES

SEQ ID NO: 30 - protein, 9 aa, Artificial sequence
  synthesized
  misc_feature: mAb041 VL-CDR3
QHSREFPWT

SEQ ID NO: 31 - protein, 116 aa, Artificial sequence
  synthesized
  misc_feature: mAb058 VH
EVQLQQSGPVLVKPGAAVKMSCKASGYIFTDYYIHWVKLSRGESLEWIGLINPYNGGTNYNQQFTGKATLTVDKSSSTAYVELNSLTSGDSAVYYCVRRTGYFDYWGQGTTLTVSS

SEQ ID NO: 32 - DNA, 348 bp, Artificial sequence
  synthesized
  misc_feature: mAb058 VH
gaggtccagctgcaacagtctggacctgtactggtgaagcctggggctgcagtgaagatgtcctgtaaggcttctggatacatattcactgactactatatacactgggtgaagctgagccgtggagagagccttgagtggattggacttattaatccttacaacggtggtactaactacaaccaacagttcacgggcaaggccacattgactgttgacaagtcctccagcacagcctacgtggagctcaacagcctgacatctggggactctgcagtctattattgtgtacggaggactggatactttgactactggggccaaggcaccactctcacagtctcctca

SEQ ID NO: 33 - protein, 5 aa, Artificial sequence
  synthesized
  misc_feature: mAb058 VH-CDR1
DYYIH

SEQ ID NO: 34 - protein, 17 aa, Artificial sequence
  synthesized
  misc_feature: mAb058 VH-CDR2
LINPYNGGTNYNQQFTG

SEQ ID NO: 35 - protein, 7 aa, Artificial sequence
  synthesized
  misc_feature: mAb058 VH-CDR3
RTGYFDY

SEQ ID NO: 36 - protein, 112 aa, Artificial sequence
  synthesized
  misc_feature: mAb058 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQTTHVPYTFGGGTKLEIK

SEQ ID NO: 37 - DNA, 336 bp, Artificial sequence
  synthesized
  misc_feature: mAb058 VL
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtccccgacaggttcagtggcagtggatcagggacagatttcacactcaagatcagcagagtggaggctgaggatctgggagtttatttctgctctcaaactacacatgttccgtacacgttcggaggggggaccaagctggaaataaaa

SEQ ID NO: 38 - protein, 16 aa, Artificial sequence
  synthesized
  misc_feature: mAb058 VL-CDR1
RSSQSLVHSNGNTYLH

SEQ ID NO: 39 - protein, 7 aa, Artificial sequence
  synthesized
  misc_feature: mAb058 VL-CDR2
KVSNRFS

SEQ ID NO: 40 - protein, 9 aa, Artificial sequence
  synthesized
  misc_feature: mAb058 VL-CDR3
SQTTHVPYT

SEQ ID NO: 41 - protein, 119 aa, Artificial sequence
  synthesized
  misc_feature: mAb034 VH
EVQLQQSGPVLVKPGASVKMSCKASGYTFTDFYMNWMRQSHGKSLEWIGVIDPYNGNTSYNQKFKGKATLTVDKSSSTAYMELNSLTSEDSAVYYCARGNYYYYAMDYWGQGTSVTVSS

SEQ ID NO: 42 - DNA, 357 bp, Artificial sequence
  synthesized
  misc_feature: mAb034 VH
gaggtccagctgcaacagtctggacctgtgctggtgaagcctggggcttcagtgaagatgtcctgtaaggcttctggatacacattcactgacttctatatgaactggatgaggcagagccatggaaagagccttgagtggattggagttattgatccttacaacggtaatactagctacaaccagaagttcaagggcaaggccacattgactgttgacaagtcctccagcacagcctacatggagctcaacagcctgacatctgaggactctgcagtctattactgtgcaagaggtaactattattactatgctatggactactggggtcaaggaacttcagtcaccgtctcctca

SEQ ID NO: 43 - protein, 5 aa, Artificial sequence
  synthesized
  misc_feature: mAb034 VH-CDR1
DFYMN

SEQ ID NO: 44 - protein, 17 aa, Artificial sequence
  synthesized
  misc_feature: mAb034 VH-CDR2
VIDPYNGNTSYNQKFKG

SEQ ID NO: 45 - protein, 10 aa, Artificial sequence
  synthesized
  misc_feature: mAb034 VH-CDR3
GNYYYYAMDY

SEQ ID NO: 46 - protein, 111 aa, Artificial sequence
  synthesized
  misc_feature: mAb034 VL
DIVLTQSPASLAVSLGQRATISCRASESVSIHGTHLMYWYQQKPGQPPKLLIYAASNLESGVPARFSGSGSETDFTLNIHPVEEEDAATFFCQQSIEDPPTFGGGTKLEIK

SEQ ID NO: 47 - DNA, 333 bp, Artificial sequence
  synthesized
  misc_feature: mAb034 VL
gacattgtgctgacccaatctccagcttctttggctgtgtctctagggcagagggccaccatctcctgcagagccagtgaaagtgtcagtattcatggtactcatttaatgtactggtaccaacagaaaccaggacagccacccaaactcctcatctatgctgcatccaacctagaatctggagtccctgccaggttcagtggcagtgggtctgagacagacttcaccctcaacatccatcctgtggaggaggaggatgctgcaacctttttctgtcagcaaagtattgaggatcctcccacgttcggaggggggaccaagctggaaataaaa

SEQ ID NO: 48 - protein, 15 aa, Artificial sequence
  synthesized
  misc_feature: mAb034 VL-CDR1
RASESVSIHGTHLMY

SEQ ID NO: 49 - protein, 7 aa, Artificial sequence
  synthesized
  misc_feature: mAb034 VL-CDR2
AASNLES

SEQ ID NO: 50 - protein, 9 aa, Artificial sequence
  synthesized
  misc_feature: mAb034 VL-CDR3
QQSIEDPPT

SEQ ID NO: 51 - protein, 117 aa, Artificial sequence
  synthesized
  misc_feature: mAb049 VH
EVQLQQSGPELVKPGASVKISCKASGHTLTDYYMNWVKQSHGKSPEWIGDINPNNDGIDYNQKFRGKAILTVDKSSSSFYMELRSLTSEDSAVYYCVGFVRGYFVSWGQGTTLTVSS

SEQ ID NO: 52 - DNA, 351 bp, Artificial sequence
  synthesized
  misc_feature: mAb049 VH
gaggtccagctgcaacaatctggacctgagctggtgaagcctggggcttcagtgaagatttcctgtaaggcttctggacacacgctcactgactactacatgaactgggtgaagcagagccatggaaagagccctgagtggattggagatattaatcctaataatgatggtattgactacaaccagaagttcaggggcaaggccatattgactgtagacaagtcctccagctcattttatatggaactccgcagtctgacatctgaggactctgcagtctattattgtgtggggttcgtacgaggctactttgtctcctggggccaaggcaccactctcacagtctcctca

SEQ ID NO: 53 - protein, 5 aa, Artificial sequence
  synthesized
  misc_feature: mAb049 VH-CDR1
DYYMN

SEQ ID NO: 54 - protein, 17 aa, Artificial sequence
  synthesized
  misc_feature: mAb049 VH-CDR2
DINPNNDGIDYNQKFRG

SEQ ID NO: 55 - protein, 8 aa, Artificial sequence
  synthesized
  misc_feature: mAb049 VH-CDR3
FVRGYFVS

SEQ ID NO: 56 - protein, 106 aa, Artificial sequence
  synthesized
  misc_feature: mAb049 VL
DIQMTQTTSSLSASLGDRVTINCRASQDISNYLNWYQQKPDGTVKLLIYYTSRLRSGVPSRFSGSGSGTDYSLTINNLDQEDFATYFCQQGNTLPWTFGGGTKLEI

SEQ ID NO: 57 - DNA, 321 bp, Artificial sequence
  synthesized
  misc_feature: mAb049 VL
gatatccagatgacacagactacatcctccctgtctgcctctctgggagacagagtcaccatcaattgcagggcaagtcaggacattagcaattatttaaactggtatcagcagaaaccagatggaactgttaaactcctgatctactacacatcaagattacgctcaggagtcccatcaaggttcagtggcagtgggtctggaacagattattctcttaccattaacaacctggaccaagaagattttgccacttacttttgtcaacagggtaatacgcttccgtggacgttcggtggaggcaccaagctggaaatcaaa

SEQ ID NO: 58 - protein, 11 aa, Artificial sequence
  synthesized
  misc_feature: mAb049 VL-CDR1
RASQDISNYLN

SEQ ID NO: 59 - protein, 7 aa, Artificial sequence
  synthesized
  misc_feature: mAb049 VL-CDR2
YTSRLRS

SEQ ID NO: 60 - protein, 9 aa, Artificial sequence
  synthesized
  misc_feature: mAb049 VL-CDR3
QQGNTLPWT

SEQ ID NO: 61 - protein, 116 aa, Artificial sequence
  synthesized
  misc_feature: mAb064 VH
QVQLKESGPGLVAPSQTLSITCSVSGFSLTNYAVSWVRQPPGKGLEWLGVMWGDGNTNYHSTLISRLSISKDNSKSQVFLKLNSLQTDDTATYYCARSSGWYFDVWGTGTTVTVSS

SEQ ID NO: 62 - DNA, 348 bp, Artificial sequence
  synthesized
  misc_feature: mAb064 VH
caggtgcagctgaaggagtcaggacctggcctggtggcgccctcacagaccctgtccatcacatgctctgtctcaggtttctcattaaccaactatgctgtaagctgggttcgccagcctccaggaaagggtctggagtggctgggagtaatgtggggtgacgggaacacaaattatcattcaactctcatatccagactgagcattagtaaggataactccaagagccaagttttcttaaaactgaacagtctacaaactgatgacacagccacgtactactgtgcccgtagtagcggctggtacttcgatgtctggggcacagggaccacggtcaccgtctcctca

SEQ ID NO: 63 - protein, 5 aa, Artificial sequence
  synthesized
  misc_feature: mAb064 VH-CDR1
NYAVS

SEQ ID NO: 64 - protein, 16 aa, Artificial sequence
  synthesized
  misc_feature: mAb064 VH-CDR2
VMWGDGNTNYHSTLIS

SEQ ID NO: 65 - protein, 8 aa, Artificial sequence
  synthesized
  misc_feature: mAb064 VH-CDR3
SSGWYFDV

SEQ ID NO: 66 - protein, 107 aa, Artificial sequence
  synthesized
  misc_feature: mAb064 VL
DIQMTQTTPSLSVTLGDRVTIRCRASQDINNYLNWYQQKPDGTVKLLIYYTSESRSGVPSRFSGSGSGTDYSLTISNLEQEDIATYFCQQGYALPPAFGGGTKLEIK

SEQ ID NO: 67 - DNA, 321 bp, Artificial sequence
  synthesized
  misc_feature: mAb064 VL
gatatccagatgacacagactacaccctccctgtctgtgactctgggagacagagtcaccatcagatgcagggcaagtcaggacattaacaattatttaaattggtatcagcagaaaccagatggaactgttaaactcctgatctactatacatcagaatcacgttcaggagtcccatcaaggttcagtggcagtgggtctggaacagattattctctcaccattagcaacctggaacaagaagatattgccacttacttttgccaacagggttatgcgcttcctccggcgttcggtggaggcaccaagctggaaataaaa

SEQ ID NO: 68 - protein, 11 aa, Artificial sequence
  synthesized
  misc_feature: mAb064 VL-CDR1
RASQDINNYLN

SEQ ID NO: 69 - protein, 7 aa, Artificial sequence
  synthesized
  misc_feature: mAb064 VL-CDR2
YTSESRS

SEQ ID NO: 70 - protein, 9 aa, Artificial sequence
  synthesized
  misc_feature: mAb064 VL-CDR3
QQGYALPPA

SEQ ID NO: 71 - protein, 118 aa, Artificial sequence
  synthesized
  misc_feature: mAb033 VH
QVQLQQSGAELVRPGTAVRVSCKASGYAFTNYFIEWVKQRPGQGLEWIGVINPGSGDTYYSEKFQGKATLTADKSSSTAYMQLSSLTSEDSAVYFCAREDDYSWFTYWGQGTLVTVSA

SEQ ID NO: 72 - DNA, 354 bp, Artificial sequence
  synthesized
  misc_feature: mAb033 VH
caggtccagctgcagcagtctggagctgagttggtaaggcctgggactgcagtgagggtgtcctgcaaggcttctggatacgccttcactaattactttatagagtgggtaaagcagaggcctggacagggccttgagtggattggagtgattaatcctggaagtggtgatacttactacagtgagaagttccagggcaaggcaacactgactgcagacaaatcctccagcactgcctacatgcagctcagcagtctgacatctgaggactctgcggtctatttctgtgcaagagaggatgattactcctggtttacttactggggccaagggactctggtcactgtctctgca

SEQ ID NO: 73 - protein, 5 aa, Artificial sequence
  synthesized
  misc_feature: mAb033 VH-CDR1
NYFIE

SEQ ID NO: 74 - protein, 17 aa, Artificial sequence
  synthesized
  misc_feature: mAb033 VH-CDR2
VINPGSGDTYYSEKFQG

SEQ ID NO: 75 - protein, 9 aa, Artificial sequence
  synthesized
  misc_feature: mAb033 VH-CDR3
EDDYSWFTY

SEQ ID NO: 76 - protein, 111 aa, Artificial sequence
  synthesized
  misc_feature: mAb033 VL
DIVLTQSPASLAVSLGQRATISCRASENVDNYGISFMHWYRQKPGQPPKLLIYRASNLESGIPARFSGSGSRTDFTLTINPVETDDVATYYCQQSNKDPYTFGGGTKLEIK

SEQ ID NO: 77 - DNA, 333 bp, Artificial sequence
  synthesized
  misc_feature: mAb033 VL
gacattgtgctgacccaatctccagcttctttggctgtgtctctagggcagagggccaccatctcctgcagagccagcgaaaatgttgataattatggcattagttttatgcactggtaccggcagaaaccaggacagccacccaaactcctcatctatcgtgcatccaacctagaatctgggatccctgccaggttcagtggcagtgggtctaggacagacttcaccctcaccattaatcctgtggagactgatgatgttgcaacctattactgtcagcaaagtaataaggatccgtacacgttcggaggggggaccaaactggaaataaaa

SEQ ID NO: 78 - protein, 15 aa, Artificial sequence
  synthesized
  misc_feature: mAb033 VL-CDR1
RASENVDNYGISFMH

SEQ ID NO: 79 - protein, 7 aa, Artificial sequence
  synthesized
  misc_feature: mAb033 VL-CDR2
RASNLES

SEQ ID NO: 80 - protein, 9 aa, Artificial sequence
  synthesized
  misc_feature: mAb033 VL-CDR3
QQSNKDPYT

SEQ ID NO: 81 - protein, 117 aa, Artificial sequence
  synthesized
  misc_feature: mAb047 VH
EVQLVESGGGLVKPGGSLKLSCAASGFTFSDYGMYWIRQAPEKGLEWVAYISNGGSSIYYADTVKGRFTVSRDNAKNTLFLQMTSLRSEDTAIYYCARGGELGFAYWGQGTLVTVSA

SEQ ID NO: 82 - DNA, 351 bp, Artificial sequence
  synthesized
  misc_feature: mAb047 VH
gaggtgcagctggtggagtctgggggaggcttagtgaagcctggagggtccctgaaactctcctgtgcagcctctggattcactttcagtgactatggaatgtactggattcgtcaggctccagagaaggggctggagtgggttgcatacattagtaatggcggtagttccatctactatgcagacacagtgaagggccgattcaccgtctccagagacaatgccaagaacaccctgttcctgcaaatgaccagtctgaggtctgaggacacggccatatattactgtgcaaggggaggagaactcgggtttgcttactggggccaagggactctggtcactgtctctgca

SEQ ID NO: 83 - protein, 5 aa, Artificial sequence
  synthesized
  misc_feature: mAb047 VH-CDR1
DYGMY

SEQ ID NO: 84 - protein, 17 aa, Artificial sequence
  synthesized
  misc_feature: mAb047 VH-CDR2
YISNGGSSIYYADTVKG

SEQ ID NO: 85 - protein, 8 aa, Artificial sequence
  synthesized
  misc_feature: mAb047 VH-CDR3
GGELGFAY

SEQ ID NO: 86 - protein, 105 aa, Artificial sequence
  synthesized
  misc_feature: mAb047 VL
DIQMTQSPASLSVSVGETVTITCRASENIYSNLAWYQQKQGKSPQLLVYAAINLVEGVPSRFSGSGSGTQYSLKINSLQSEDFGSYFCQHFWGLPYTFGGGTKLE

SEQ ID NO: 87 - DNA, 321 bp, Artificial sequence
  synthesized
  misc_feature: mAb047 VL
gacatccagatgactcagtctccagcctccctatctgtatctgtgggagaaactgtcaccatcacgtgtcgagcaagtgaaaatatttacagtaatctagcatggtatcagcagaaacagggaaaatctcctcagctcctggtctatgctgcaataaatttagtagaaggtgtgccatcaaggttcagtggcagtggatcgggcacgcagtattccctcaagatcaacagcctgcagtctgaagattttgggagttatttctgtcaacatttttggggtcttccgtacacgttcggaggggggaccaagctggaaagaaat

SEQ ID NO: 88 - protein, 11 aa, Artificial sequence
  synthesized
  misc_feature: mAb047 VL-CDR1
RASENIYSNLA

SEQ ID NO: 89 - protein, 7 aa, Artificial sequence
  synthesized
  misc_feature: mAb047 VL-CDR2
AAINLVE

SEQ ID NO: 90 - protein, 9 aa, Artificial sequence
  synthesized
  misc_feature: mAb047 VL-CDR3
QHFWGLPYT

SEQ ID NO: 91 - protein, 112 aa, Artificial sequence
  synthesized
  misc_feature: mAb055 VH
QVQLKQSGPGLLQPSQSLSITCTVSGFSLDEYGVHWFRQSPGKGLEWLGVIWSGGSTDYNAAFISRLSIRKDNSKSQIFFNMNSLQADDTAIYYCVREEWEYWGQGTTLTVS

SEQ ID NO: 92 - DNA, 339 bp, Artificial sequence
  synthesized
  misc_feature: mAb055 VH
caggtgcagctgaagcagtcaggacctggcctactgcagccttcacagagcctgtccatcacctgcacagtctctggtttctcgttagatgagtatggtgtacattggtttcgtcagtctccaggaaagggtctggagtggctgggagtgatatggagtggtggaagcacagactacaatgcagctttcatatccagactgagcatcagaaaggacaattccaagagccaaattttctttaatatgaacagtctacaagctgatgacacagccatatattactgtgtcagagaggaatgggaatattggggccaaggcacaactctcacagtctcctca

SEQ ID NO: 93 - protein, 5 aa, Artificial sequence
  synthesized
  misc_feature: mAb055 VH-CDR1
EYGVH

SEQ ID NO: 94 - protein, 16 aa, Artificial sequence
  synthesized
  misc_feature: mAb055 VH-CDR2
VIWSGGSTDYNAAFIS

SEQ ID NO: 95 - protein, 5 aa, Artificial sequence
  synthesized
  misc_feature: mAb055 VH-CDR3
EEWEY

SEQ ID NO: 96 - protein, 107 aa, Artificial sequence
  synthesized
  misc_feature: mAb055 VL
DIQMTQTTSSLSASLGDRVTISCRPSQDIDNYLNWFQQRPDGTIKLLIYYTSRLHSGVPSRFSGSGSGTDYSLTINNLEQEDIATYFCQQAHTLPYTFGGGTKLEIK

SEQ ID NO: 97 - DNA, 321 bp, Artificial sequence
  synthesized
  misc_feature: mAb055 VL
gatatccagatgacacagactacatcctccctgtctgcctctctgggagacagagtcaccatcagttgcaggccaagtcaggacattgacaattatttaaactggtttcagcagagaccagatggaactattaaactcctgatctattatacatcaagactacactcaggagtcccatcgaggttcagtggcagtgggtctggaacagattattctctcaccattaacaacctggaacaagaagatattgccacttacttttgccaacaggctcatacgcttccgtacacgttcggaggggggaccaagctggaaataaaa

SEQ ID NO: 98 - protein, 11 aa, Artificial sequence
  synthesized
  misc_feature: mAb055 VL-CDR1
RPSQDIDNYLN

SEQ ID NO: 99 - protein, 7 aa, Artificial sequence
  synthesized
  misc_feature: mAb055 VL-CDR2
YTSRLHS

SEQ ID NO: 100 - protein, 9 aa, Artificial sequence
  synthesized
  misc_feature: mAb055 VL-CDR3
QQAHTLPYT

SEQ ID NO: 101 - protein, 116 aa, Artificial sequence
  synthesized
  misc_feature: Humab035-24-VH
QVQLVQSGAEVKKPGASVKVSCKASDYTFTDYEMHWVRQAPGQGLEWIGAIDPETGGTAYNQKFKDRVTMTADTSTSTVYMELSSLRSEDTAVYYCAKTGYQFDYWGQGTTVTVSS

SEQ ID NO: 102 - DNA, 348 bp, Artificial sequence
  synthesized
  misc_feature: Humab035-24-VH
caggtgcagctggtgcagtcaggagcagaagtgaagaagccaggcgcaagcgtgaaggtgtcttgcaaggccagcgactacaccttcaccgactacgagatgcattgggtccggcaggctccaggacagggactcgagtggattggcgctatcgacccagagacaggcggcacagcctataaccagaagttcaaggaccgcgtgaccatgaccgccgatacaagcaccagcaccgtgtacatggagctgagcagcctgagaagcgaggacaccgccgtgtactattgcgccaagaccggctaccagttcgactattggggccagggaaccacagtgacagtgtccagc

SEQ ID NO: 103 - protein, 107 aa, Artificial sequence
  synthesized
  misc_feature: Humab035-24-VL
DIQMTQSPSSVSASVGDRVTITCKASQNVGSNVAWYQQKPGKAPKALIYSASYRYSGVPSRFSGSGSGTDFTLTISSLQPEDFATYYCQQYNSHPYTFGQGTKLEIK

SEQ ID NO: 104 - DNA, 321 bp, Artificial sequence
  synthesized
  misc_feature: Humab035-24-VL
gacatccagatgacccagagccctagcagcgtgtccgctagcgtgggagatagagtgaccatcacttgcaaggccagccagaacgtgggcagcaacgtggcttggtatcagcagaagccagggaaggcccctaaggccctgatctacagcgccagctacagatacagcggcgtgccttctagattcagcggcagcggaagcggcaccgatttcaccctgaccatcagcagcctgcagccagaggacttcgccacctactactgccagcagtacaacagccacccctacaccttcggacagggcaccaagctggagatcaag

(19) United States
(12) Patent Application Publication
(10) Pub. No.: US 2022/0017629 A1
(43) Pub. Date: Jan. 20, 2022

(54) OX40 ANTIBODY, PREPARATION METHOD THEREOF AND USE THEREOF

(71) Applicants: SHANGHAI PHARMAEXPLORER CO., LTD., Shanghai (CN); PHARMAEXPLORER LIMITED, Tortola (VG)

(72) Inventors: Xiaojuan CHAI, Shanghai (CN); Wenfang XIN, Shanghai (CN); Yanyun CAO, Shanghai (CN); Hui XU, Shanghai (CN); Yu ZHANG, Shanghai (CN); Ke XIA, Shanghai (CN); Qing DUAN, Shanghai (CN); Tatchi Teddy YANG, Shanghai (CN); Lile LIU, Shanghai (CN)

(21) Appl. No.: 17/295,225

(22) PCT Filed: Nov. 19, 2019
(86) PCT No.: PCT/CN2019/119515

§ 371 Date: Sep. 28, 2021

(30) Foreign Application Priority Data
Nov. 20, 2018 (CN) 201811386274.3

(51) Int. Cl. C07K 16/28; A61K 31/337; A61K 31/282; A61K 39/395; A61P 35/00

(52) U.S. Cl. CPC C07K 16/2878; A61K 31/337; A61K 2039/505; A61K 39/3955; A61P 35/00; A61K 31/282

(57) ABSTRACT

Provided are a murine or human-mouse chimeric antibody targeting OX40 and a preparation method therefor, wherein the antibody binds to the OX40 antigen and has an anti-tumor activity and other activities.

### TECHNICAL FIELD

The present invention belongs to the field of biomedicine, in particular to an OX40 antibody, preparation method thereof and use thereof.

### BACKGROUND

OX40 (also known as CD134, TNFRSF4 or ACT35) is a tumor necrosis factor receptor superfamily (TNFRSF) member and a type I transmembrane glycoprotein. OX40 is mainly expressed in activated CD4+ T cell and CD8+ T cell, regulatory T cell (Treg) and natural killer cell (NK). The ligand of OX40 in the body is OX40L (also known as CD152 or TNFSF4), which exists as a trimer. OX40/OX40L signal plays a very important role in the activation and proliferation of T cells. Studies have shown that OX40 antibody can effectively promote the activation and proliferation of T cells, block the immunosuppressive effect of Treg cells, and activate the immune system.

Clinical sample studies have found that OX40 is expressed in tumor infiltrating lymphocytes (TILs), such as breast cancer, melanoma, B-cell lymphoma, head and neck cancer, and colon cancer.

In mice tumor models such as CT26 colon cancer, N2C breast cancer, A20 lymphoma, and B16-F10 melanoma, the mouse OX40 antibody (OX86) can effectively control the growth of tumors and improve the survival rate of mice. In addition, the combination of OX40 antibody and other immune checkpoint-related antibodies, such as 4-1BB antibody, PD-L1 antibody or CTLA-4 antibody, has good anti-tumor activity and can significantly improve the survival rate. Therefore, the development of OX40 antibody has very important clinical significance.

At present, there is no better OX40 antibody that can properly regulate the immune system and be used in tumor immunotherapy, and achieves the effect of high antibody titer and low toxic side effects. Therefore, it is urgent to solve the above problems.

### SUMMARY OF THE INVENTION

In order to overcome the shortcomings of current lack of superior OX40 antibodies, the present invention provides an OX40 antibody with high affinity and strong specificity, and a preparation method thereof and use thereof.

In a first aspect of the present invention, it provides a heavy chain variable region of an antibody having a complementarity determining region CDR selected from the group consisting of:

a VH-CDR1 as shown in SEQ ID NO. 10n+3,

a VH-CDR2 as shown in SEQ ID NO. 10n+4, and

a VH-CDR3 as shown in SEQ ID NO. 10n+5;

wherein each n is independently 0, 1, 2, 3, 4, 5, 6, 7, 8 or 9;

wherein any one of the above amino acid sequences further includes a derivative sequence that is optionally added, deleted, modified, and/or substituted for at least one amino acid and can retain the binding affinity of OX40.

In another preferred embodiment, the heavy chain variable region has the amino acid sequence as shown in SEQ ID NO. 101 or 10n+1, wherein n is 0, 1, 2, 3, 4, 5, 6, 7, 8, or 9.

In another preferred embodiment, the heavy chain variable region has the amino acid sequence as shown in SEQ ID NO. 101.

In another preferred embodiment, the heavy chain variable region has the amino acid sequence as shown in SEQ ID NO. 1.

In another preferred embodiment, the heavy chain variable region has the amino acid sequence as shown in SEQ ID NO. 11.

In another preferred embodiment, the heavy chain variable region has the amino acid sequence as shown in SEQ ID NO. 21.

In another preferred embodiment, the heavy chain variable region has the amino acid sequence as shown in SEQ ID NO. 31.

In a second aspect of the present invention, it provides a heavy chain of an antibody having the heavy chain variable region of claim 1.

In another preferred embodiment, the heavy chain further comprises a heavy chain constant region.

In another preferred embodiment, the heavy chain constant region is of human or murine origin.

In another preferred embodiment, the heavy chain constant region is a human antibody heavy chain IgG1 constant region.

In a third aspect of the present invention, it provides a light chain variable region of an antibody having a complementarity determining region CDR selected from the group consisting of:

a VL-CDR1 as shown in SEQ ID NO. 10n+8,

a VL-CDR2 as shown in SEQ ID NO. 10n+9, and

a VL-CDR3 as shown in SEQ ID NO. 10n+10;

wherein each n is independently 0, 1, 2, 3, 4, 5, 6, 7, 8 or 9;

wherein any one of the above amino acid sequences further includes a derivative sequence that is optionally added, deleted, modified, and/or substituted for at least one amino acid and can retain the binding affinity of OX40.

In another preferred embodiment, the light chain variable region has the amino acid sequence as shown in SEQ ID NO. 103 or 10n+6, wherein n is 0, 1, 2, 3, 4, 5, 6, 7, 8, or 9.

In another preferred embodiment, the light chain variable region has the amino acid sequence as shown in SEQ ID NO. 103.

In another preferred embodiment, the light chain variable region has the amino acid sequence as shown in SEQ ID NO. 6.

In another preferred embodiment, the light chain variable region has the amino acid sequence as shown in SEQ ID NO. 16.

In another preferred embodiment, the light chain variable region has the amino acid sequence as shown in SEQ ID NO. 26.

In another preferred embodiment, the light chain variable region has the amino acid sequence as shown in SEQ ID NO. 36.

In a fourth aspect of the present invention, it provides a light chain of an antibody having the light chain variable region of claim 3.

In another preferred embodiment, the light chain further comprises a light chain constant region.

In another preferred embodiment, the light chain constant region is of human or murine origin.

In another preferred embodiment, the light chain constant region is a human antibody light chain kappa constant region.

In a fifth aspect of the present invention, it provides an antibody having:

(1) a heavy chain variable region according to the first aspect of the present invention; and/or

(2) a light chain variable region according to the third aspect of the present invention;

or the antibody has the heavy chain according to the second aspect of the present invention; and/or the light chain according to the fourth aspect of the present invention,

wherein any one of the above amino acid sequences further includes a derivative sequence that is optionally added, deleted, modified, and/or substituted for at least one amino acid and can retain the binding affinity of OX40.

In another preferred embodiment, the amino acid sequence of any of the above-mentioned CDRs contains a derivative CDR sequence of 1, 2, or 3 amino acids that has been added, deleted, modified, and/or substituted, and so that the derivative antibody composed of VH and VL containing the derivative CDR sequence can retain the affinity for binding to OX40.

In another preferred embodiment, the ratio (F1/F0) of the affinity F1 of the derivatized antibody binding to OX40 and the affinity F0 of the corresponding non-derivatized antibody binding to OX40 is 0.5-2, preferably 0.7-1.5, and more preferably 0.8-1.2.

In another preferred embodiment, the number of added, deleted, modified and/or substituted amino acids is 1-5 (such as 1-3, preferably 1-2, more preferably 1).

In another preferred embodiment, the derivative sequence that has been added, deleted, modified and/or substituted at least one amino acid and can retain OX40 binding affinity is an amino acid sequence with homology or sequence identity of at least 96%.

In another preferred embodiment, the antibody further comprises a heavy chain constant region and/or light chain constant region.

In another preferred embodiment, the heavy chain constant region is of human origin, and/or the light chain constant region is of human origin.

In another preferred embodiment, the heavy chain constant region is a human antibody heavy chain IgG1 constant region, and the light chain constant region is a human antibody light chain kappa constant region.

In another preferred embodiment, the heavy chain variable region of the antibody further comprises a human framework region, and/or the light chain variable region of the antibody further comprises a human framework region.

In another preferred embodiment, the heavy chain variable region of the antibody further comprises a framework region of murine origin, and/or the light chain variable region of the antibody further comprises a framework region of murine origin.

In another preferred embodiment, the antibody is selected from the group consisting of an animal-derived antibody, chimeric antibody, humanized antibody, fully human antibody, and a combination thereof.

In another preferred embodiment, the ratio (Z1/Z0) of the immunogenicity Z1 of the chimeric antibody in humans and the immunogenicity Z0 of non-chimeric antibody (such as murine antibodies) in humans is 0-0.5, preferably 0-0.2, more preferably 0-0.05 (e.g., 0.001-0.05).

In another preferred embodiment, the antibody is a partially or fully humanized, or fully human monoclonal antibody.

In another preferred embodiment, the antibody is a double-chain antibody or a single-chain antibody.

In another preferred embodiment, the antibody is a full-length protein of an antibody, or an antigen binding fragment.

In another preferred embodiment, the antibody is a bispecific antibody or a multispecific antibody.

In another preferred embodiment, the antibody is in the form of a drug conjugate.

In another preferred embodiment, the antibody has one or more characteristics selected from the group consisting of:

(a) inhibiting the migration or metastasis of tumor cells;

(b) inhibiting tumor growth.

In another preferred embodiment, the antibody has a heavy chain variable region according to the first aspect of the present invention and a light chain variable region according to the third aspect of the present invention;

wherein the heavy chain variable region and the light chain variable region comprise a CDR selected from the group consisting of:

| VH-CDR1 | VH-CDR2 | VH-CDR3 | VL-CDR1 | VL-CDR2 | VL-CDR3 |
|---|---|---|---|---|---|
| Sequence | Sequence | Sequence | Sequence | Sequence | Sequence |
| Number | Number | Number | Number | Number | Number |
| 3 | 4 | 5 | 8 | 9 | 10 |
| 13 | 14 | 15 | 18 | 19 | 20 |
| 23 | 24 | 25 | 28 | 29 | 30 |
| 33 | 34 | 35 | 38 | 39 | 40 |
| 43 | 44 | 45 | 48 | 49 | 50 |
| 53 | 54 | 55 | 58 | 59 | 60 |
| 63 | 64 | 65 | 68 | 69 | 70 |
| 73 | 74 | 75 | 78 | 79 | 80 |
| 83 | 84 | 85 | 88 | 89 | 90 |
| 93 | 94 | 95 | 98 | 99 | 100 |

wherein any one of the above amino acid sequences also includes a derivative sequence that is optionally added, deleted, modified, and/or substituted for at least one amino acid and can retain the binding affinity of OX40.

In another preferred embodiment, the antibody has a heavy chain variable region according to the first aspect of the present invention and a light chain variable region according to the third aspect of the present invention;

Wherein the heavy chain variable region comprises the following three complementarity determining region CDRs:

a VH-CDR1 as shown in SEQ ID NO. 3,

a VH-CDR2 as shown in SEQ ID NO. 4, and

a VH-CDR3 as shown in SEQ ID NO. 5;

the light chain variable region comprises the following three complementarity determining region CDRs:

a VL-CDR1 as shown in SEQ ID NO. 8,

a VL-CDR2 as shown in SEQ ID NO. 9, and

a VL-CDR3 as shown in SEQ ID NO. 10;

or

the heavy chain variable region comprises the following three complementarity determining region CDRs:

a VH-CDR1 as shown in SEQ ID NO. 13,

a VH-CDR2 as shown in SEQ ID NO. 14, and

a VH-CDR3 as shown in SEQ ID NO. 15;

The light chain variable region comprises the following three complementarity determining region CDRs:

a VL-CDR1 as shown in SEQ ID NO. 18,

a VL-CDR2 as shown in SEQ ID NO. 19, and

a VL-CDR3 as shown in SEQ ID NO. 20;

or

the heavy chain variable region comprises the following three complementarity determining region CDRs:

a VH-CDR1 as shown in SEQ ID NO. 23,

a VH-CDR2 as shown in SEQ ID NO. 24, and

a VH-CDR3 as shown in SEQ ID NO. 25;

the light chain variable region comprises the following three complementarity determining region CDRs:

a VL-CDR1 as shown in SEQ ID NO. 28,

a VL-CDR2 as shown in SEQ ID NO. 29, and

a VL-CDR3 as shown in SEQ ID NO. 30;

or

the heavy chain variable region comprises the following three complementarity determining region CDRs:

a VH-CDR1 as shown in SEQ ID NO. 33,

a VH-CDR2 as shown in SEQ ID NO. 34, and

a VH-CDR3 as shown in SEQ ID NO. 35;

The light chain variable region comprises the following three complementarity determining region CDRs:

a VL-CDR1 as shown in SEQ ID NO. 38,

a VL-CDR2 as shown in SEQ ID NO. 39, and

a VL-CDR3 as shown in SEQ ID NO. 40.

In another preferred embodiment, the heavy chain variable region of the antibody contains the amino acid sequence as shown in SEQ ID NO. 101, 1, 11, or 21; and/or the light chain variable region of the antibody contains the amino acid sequence as shown in SEQ ID NO. 103, 6, 16 or 26.

In another preferred embodiment, the heavy chain variable region of the antibody comprises the amino acid sequence as shown in SEQ ID NO. 101; and the light chain variable region of the antibody comprises the amino acid sequence as shown in SEQ ID NO. 103.

In another preferred embodiment, the heavy chain variable region of the antibody comprises the amino acid sequence as shown in SEQ ID NO. 1; and the light chain variable region of the antibody comprises the amino acid sequence as shown in SEQ ID NO. 6.

In another preferred embodiment, the heavy chain variable region of the antibody comprises the amino acid sequence as shown in SEQ ID NO. 11; and the light chain variable region of the antibody comprises the amino acid sequence as shown in SEQ ID NO. 16.

In another preferred embodiment, the heavy chain variable region of the antibody comprises the amino acid sequence as shown in SEQ ID NO. 21; and the light chain variable region of the antibody comprises the amino acid sequence as shown in SEQ ID NO. 26.

In another preferred embodiment, the heavy chain variable region of the antibody comprises the amino acid sequence as shown in SEQ ID NO. 31; and the light chain variable region of the antibody comprises the amino acid sequence as shown in SEQ ID NO. 36.

In another preferred embodiment, the antibody is selected from the group consisting of:

| Antibody | | VH sequence | VL Sequence |
|---|---|---|---|
| number | Clone | Number | Number |
| 1 | Humab035-24 | 101 | 103 |
| 2 | Mab035 | 1 | 6 |
| 3 | Mab053 | 11 | 16 |
| 4 | Mab041 | 21 | 26 |
| 5 | Mab058 | 31 | 36 |
| 6 | Mab034 | 41 | 46 |
| 7 | Mab049 | 51 | 56 |
| 8 | Mab064 | 61 | 66 |
| 9 | Mab033 | 71 | 76 |
| 10 | Mab047 | 81 | 86 |
| 11 | Mab055 | 91 | 96. |

In another preferred embodiment, the amino acid sequence of the variable region of the heavy chain has at least 80%, 85%, 90%, 91%, 92%, 93%, 94%, 95%, 96%, 97%, 98% or 99% sequence homology or sequence identity with the amino acid sequence as shown in SEQ ID NO. 101, 1, 11 or 21 in the sequence listing.

In another preferred embodiment, the amino acid sequence of the light chain variable region has at least 80%, 85%, 90%, 91%, 92%, 93%, 94%, 95%, 96%, 97%, 98% or 99% sequence homology or sequence identity with the amino acid sequence as shown in SEQ ID NO. 103, 6, 16 or 26 in the sequence listing.

In a sixth aspect of the present invention, it provides a recombinant protein comprising:

(i) a heavy chain variable region according to the first aspect of the present invention, a heavy chain according to the second aspect of the present invention, a light chain variable region according to the third aspect of the present invention, a light chain according to the fourth aspect of the present invention, or an antibody according to the fifth aspect of the present invention; and

(ii) an optional tag sequence to assist in expression and/or purification.

In another preferred embodiment, the tag sequence comprises 6His tag.

In another preferred embodiment, the recombinant protein (or polypeptide) comprises a fusion protein.

In another preferred embodiment, the recombinant protein is a monomer, dimer, or multimer.

In another preferred embodiment, the recombinant protein comprises:

(i) an antibody selected from the group consisting of,

| Antibody | | VH Sequence | VL Sequence |
|---|---|---|---|
| number | Clone | Number | Number |
| 1 | Humab035-24 | 101 | 103 |
| 2 | Mab035 | 1 | 6 |
| 3 | Mab053 | 11 | 16 |
| 4 | Mab041 | 21 | 26 |
| 5 | Mab058 | 31 | 36 |
| 6 | Mab034 | 41 | 46 |
| 7 | Mab049 | 51 | 56 |
| 8 | Mab064 | 61 | 66 |
| 9 | Mab033 | 71 | 76 |
| 10 | Mab047 | 81 | 86 |
| 11 | Mab055 | 91 | 96 |

and

(ii) an optional tag sequence to assist in expression and/or purification.

In a seventh aspect of the present invention, it provides a polynucleotide encoding a polypeptide selected from the group consisting of:

(1) a heavy chain variable region according to the first aspect of the present invention, a heavy chain according to the second aspect of the present invention, a light chain variable region according to the third aspect of the present invention, a light chain according to the fourth aspect of the present invention, or an antibody according to the fifth aspect of the present invention; and

(2) the recombinant protein according to the sixth aspect of the present invention.

In another preferred embodiment, the polynucleotide encoding the variable region of the heavy chain is shown in SEQ ID NO. 102, 2, 12, 22, 32, 42, 52, 62, 72, 82, or 92; and/or, the polynucleotide encoding the variable region of the light chain is shown in SEQ ID NO. 104, 7, 17, 27, 37, 47, 57, 67, 77, 87 or 97.

In another preferred embodiment, the polynucleotide encoding the heavy chain variable region sequence and the polynucleotide encoding the light chain variable region sequence are selected from the group consisting of:

| | Sequence numbering | Sequence numbering |
|---|---|---|
| | of polynucleotide | of polynucleotide |
| Clone | encoding VH | encoding VL |
| Humab035-24 | 102 | 104 |
| Mab035 | 2 | 7 |
| Mab053 | 12 | 17 |
| Mab041 | 22 | 27 |
| Mab058 | 32 | 37 |
| Mab034 | 42 | 47 |
| Mab049 | 52 | 57 |
| Mab064 | 62 | 67 |
| Mab033 | 72 | 77 |
| Mab047 | 82 | 87 |
| Mab055 | 92 | 97. |

In an eighth aspect of the present invention, it provides a vector comprising the polynucleotide according to any one of the seventh aspects of the present invention.

In another preferred embodiment, the vector includes: a bacterial plasmid, bacteriophage, yeast plasmid, plant cell virus, mammalian cell virus such as adenovirus, retrovirus, or other vectors.

In a ninth aspect of the present invention, it provides a genetically engineered host cell comprising the vector according to the eighth aspect of the present invention or the genome integrated with the polynucleotide according to the seventh aspect of the present invention.

In a tenth aspect of the present invention, it provides an antibody conjugate comprising:

(a) an antibody portion, which is selected from the group consisting of a heavy chain variable region according to the first aspect of the present invention, a heavy chain according to the second aspect of the present invention, a light chain variable region according to the third aspect of the present invention, a light chain according to the fourth aspect of the present invention, or an antibody according to the fifth aspect of the present invention, and a combination thereof; and

(b) a coupling portion coupled to the antibody portion, the coupling portion is selected from the group consisting of a detectable marker, drug, toxin, cytokine, radionuclide, enzyme, and a combination thereof.

In another preferred embodiment, the antibody portion is coupled to the coupling portion by a chemical bond or linker.

In an eleventh aspect of the present invention, it provides an immune cell that expresses or is exposed outside the cell membrane with the antibody according to the fifth aspect of the present invention.

In another preferred embodiment, the immune cell comprises a NK cell, T cell.

In another preferred embodiment, the immune cell is derived from human or non-human mammals (such as mice).

In a twelfth aspect of the present invention, it provides a pharmaceutical composition comprising:

(i) an active ingredient, the active ingredient is selected from the group consisting of: the heavy chain variable region according to the first aspect of the present invention, the heavy chain according to the second aspect of the present invention, the light chain variable region according to the third aspect of the present invention, the light chain according to the fourth aspect of the present invention, or the antibody according to the fifth aspect of the present invention, the recombinant protein according to the sixth aspect of the present invention, the antibody conjugate according to the tenth aspect of the present invention, the immune cell according to the eleventh aspect of the present invention, and a combination thereof; and

(ii) a pharmaceutically acceptable carrier.

In another preferred embodiment, the pharmaceutical composition is a liquid formulation.

In another preferred embodiment, the pharmaceutical composition is an injection.

In another preferred embodiment, the pharmaceutical composition comprises 0.01-99.99% of the antibody according to the fifth aspect of the present invention, the recombinant protein according to the sixth aspect of the present invention, and the antibody conjugate according to the tenth aspect of the present invention, the immune cell according to the eleventh aspect of the present invention, and a combination thereof, and 0.01-99.99% of the pharmaceutical carrier, and the percentage is the mass percentage of the pharmaceutical composition.

In a thirteenth aspect of the present invention, it provides a use of an active ingredient selected from the group consisting of the heavy chain variable region according to the first aspect of the present invention, and the heavy chain according to the second aspect of the present invention, the light chain variable region according to the third aspect of the present invention, the light chain according to the fourth aspect of the present invention, or the antibody according to the fifth aspect of the present invention, the recombinant protein according to the sixth aspect of the present invention, the antibody conjugate according to the tenth aspect of the present invention, the immune cell according to the eleventh aspect of the present invention, and a combination thereof, wherein the active ingredient is used for (a) preparing a diagnostic reagent or kit; and/or (b) preparing a drug for preventing and/or treating diseases related to abnormal expression or function of OX40.

In another preferred embodiment, the diagnostic reagent is a detective slip or a detection plate.

In another preferred embodiment, the diseases related to the abnormal expression or function of OX40 are selected from the group consisting of: cancer, autoimmune diseases.

In another preferred embodiment, the cancer is selected from the group consisting of: breast cancer, melanoma, B-cell lymphoma, head and neck cancer, colon cancer, prostate cancer, lung cancer.

In another preferred embodiment, the diagnostic reagent or kit is used for:

(1) detecting the OX40 protein in a sample; and/or

(2) detecting endogenous OX40 protein in a tumor cell; and/or

(3) detecting a tumor cell expressing OX40 protein;

the drug is used to prevent and/or treat diseases related to abnormal expression or function of OX40, and the diseases related to abnormal expression or function of OX40 are selected from the group consisting of: cancer, autoimmune diseases.

In another preferred embodiment, the cancer is selected from the group consisting of: breast cancer, melanoma, B-cell lymphoma, head and neck cancer, colon cancer, prostate cancer, lung cancer.

In another preferred embodiment, the antibody is in the form of a drug conjugate (ADC).

In another preferred embodiment, the diagnostic reagent or kit is used for diagnosing OX40 related diseases.

In another preferred embodiment, the diagnostic reagent or kit is used for detecting OX40 protein in a sample.

In a fourteenth aspect of the present invention, it provides a method for detecting (including diagnostic or non-diagnostic) OX40 protein in a sample in vitro, the method comprising the steps:

(1) in vitro, contacting the sample with the antibody according to the fifth aspect of the present invention;

(2) detecting the formation of an antigen-antibody complex, wherein the formation of a complex indicates the presence of OX40 protein in the sample.

In a fifteenth aspect of the present invention, it provides a composition for detecting OX40 protein in a sample in vitro, which comprises the antibody according to the fifth aspect of the present invention, the recombinant protein according to the sixth aspect of the present invention, the antibody conjugate according to the tenth aspect of the present invention, the immune cell according to the eleventh aspect of the present invention, and a combination thereof as an active ingredient.

In a sixteenth aspect of the present invention, it provides a detection plate comprising a substrate (support plate) and a test strip, and the test strip contains the antibody according to the fifth aspect of the present invention, the recombinant protein according to the sixth aspect of the present invention, and the antibody conjugate according to the tenth aspect of the present invention, the immune cell according to the eleventh aspect of the present invention, and a combination thereof.

In a seventeenth aspect of the present invention, it provides a kit comprising:

(1) a first container containing the antibody of the present invention; and/or

(2) a second container containing a secondary antibody against the antibody of the present invention;

or,

The kit contains the detection plate according to the sixteenth aspect of the present invention.

In an eighteenth aspect of the present invention, it provides a method for preparing a recombinant polypeptide, the method comprising:

(a) culturing the host cell according to the ninth aspect of the present invention under conditions suitable for expression;

(b) isolating the recombinant polypeptide from the culture, the recombinant polypeptide being an antibody according to the fifth aspect of the present invention or a recombinant protein according to the sixth aspect of the present invention.

In a nineteenth aspect of the present invention, it provides a pharmaceutical combination comprising:

(I) a first active ingredient comprising the antibody 1 according to the fifth aspect of the present invention, or the recombinant protein according to the sixth aspect of the present invention, or the antibody conjugate according to the tenth aspect of the present invention, or the immune cell according to the eleventh aspect of the present invention, or the pharmaceutical composition according to the twelfth aspect of the present invention, and a combination thereof;

(ii) a second active ingredient comprising a second antibody, or a chemotherapeutic agent.

In another preferred embodiment, the second antibody is selected from the group consisting of: CTLA4 antibody, PD-1 antibody, PD-L1 antibody, 4-1BB antibody.

In another preferred embodiment, the chemotherapeutic agent is selected from the group consisting of docetaxel, carboplatin, and a combination thereof.

In a twentieth aspect of the present invention, it provides a use of the combination of the antibody of the fifth aspect of the present invention, or the recombinant protein according to the sixth aspect of the present invention, or the antibody conjugate according to the tenth aspect of the present invention, or the immune cell according to the eleventh aspect of the present invention, and/or the pharmaceutical composition according to the twelfth aspect of the present invention and a second antibody or a chemotherapeutic agent in the preparation of a medicament for treating diseases related to abnormal OX40 expression or function.

In another preferred embodiment, the second antibody is selected from the group consisting of: CTLA4 antibody, PD-1 antibody, PD-L1 antibody, 4-1BB antibody.

In a twenty-first aspect of the present invention, it provides a method for treating diseases related to abnormal expression or function of OX40, administering to a subject in need an effective amount of the antibody according to the fifth aspect of the present invention, or the recombinant protein according to the sixth aspect of the present invention, or the antibody conjugate according to the tenth aspect of the present invention, or the immune cell according to the eleventh aspect of the present invention, or the pharmaceutical composition according to the twelfth aspect of the present invention, and a combination thereof.

In another preferred embodiment, the diseases related to the abnormal expression or function of OX40 are selected from the group consisting of cancer and autoimmune diseases.

In another preferred embodiment, the cancer is selected from the group consisting of: breast cancer, melanoma, B-cell lymphoma, head and neck cancer, colon cancer, prostate cancer, lung cancer.

In another preferred embodiment, the method further comprises before, during, and/or after the administration of the first active ingredient, a safe and effective amount of the second antibody is administered to the subject.

In another preferred embodiment, the second antibody is selected from the group consisting of: PD-1 antibody, CTLA4 antibody, PD-L1 antibody, 4-1BB antibody.

It should be understood that, within the scope of the present invention, the technical features specifically described above and below (such as the Examples) can be combined with each other, thereby constituting a new or preferred technical solution which needs not be described one by one.

### DESCRIPTION OF DRAWINGS

FIG. 1 shows the binding activity of OX40 ECD-hFc and OX40L protein. Wherein The X-axis represents the concentration of OX40L protein, and the Y-axis represents the OD450 nm value.

FIG. 2 shows the results of FACS analysis of the HEK293-hOX40 cell line.

FIG. 3 shows the results of FACS analysis of the CHOK1-hOX40 cell line.

FIG. 4 shows the results of FACS analysis of the CHOK1-cOX40 cell line.

FIG. 5 shows the result of ELISA detection of the serum antibody titer of mice after immunization with OX40 ECD-hFc protein.

FIG. 6 shows the results of FACS detection of the serum antibody titer of mice after immunization with OX40 ECD-hFc protein.

FIG. 7 shows the results of FACS detection of antibody titer in mouse serum after HEK293-hOX40 cell immunization.

FIG. 8 shows the results of FACS detection of antibody titer in mouse serum after pCP-hOX40 plasmid immunization.

FIGS. 9a and 9b show the FACS detection of the binding reaction between OX40 antibody and CHOK1-hOX40.

FIGS. 10a and 10b show the FACS detection of the binding reaction between OX40 antibody and CHOK1-cOX40.

FIG. 11 shows the FACS detection of hOX40 expression in Jurkat-NF-kB Luc-hOX40 cells.

FIG. 12 shows the luciferase method to detect the expression of NF-kB RE-luciferase in Jurkat-NF-kB Luc-hOX40 cells.

FIGS. 13a and 13b show the NF-kB reporter gene experiment to detect the activation of the OX40 antibody on the NF-kB signaling pathway, wherein blank is a blank control; Ionomycin is ionomycin (IONO).

FIG. 14 shows the results of FACS analysis of the CHOK1-hOX40L cell line.

FIGS. 15a and 15b show the FACS detection of the inhibitory effect of OX40 antibody on the binding of OX40 and OX40L proteins.

FIG. 16 shows the FACS detection of the binding reaction of OX40 human-mouse chimeric antibody and CHOK1-hOX40.

FIG. 17 shows the FACS detection of the binding reaction of OX40 human-mouse chimeric antibody and CHOK1-cOX40.

FIG. 18 shows the NF-kB reporter gene experiment to detect the activation of the OX40 human-mouse chimeric antibody on the NF-kB signaling pathway.

FIGS. 19a and 19b show the body weight changes of mice in each group after administration.

FIG. 20 shows the changes in tumor volume of mice in each group after administration.

FIG. 21 shows the survival curve of mice in each group after administration.

FIG. 22 shows the FACS detection of the binding reaction of OX40 humanized antibody with CHOK1-hOX40.

FIG. 23 shows the NF-kB reporter gene experiment to detect the activation of the OX40 humanized antibody on the NF-kB signaling pathway.

FIG. 24 is a T cell activation test to detect the activation of OX40 humanized antibody on memory CD4 positive T cells.

### DETAILED DESCRIPTION

Through extensive and in-depth research, the inventors of the present invention have used human OX40 protein, cell lines stably expressing OX40 protein, and plasmids containing cDNA encoding OX40 protein as immunogens. Using hybridoma technology, they have unexpectedly obtained a group of mouse-derived or human-mouse chimeric OX40 antibodies with a completely new amino acid sequence (for example, mab035, mab053, mab041, and mab058). The OX40 antibody of the present invention is an OX40 agonistic antibody with excellent biological characteristics. It has high affinity with human OX40 protein and has similar binding ability with monkey OX40 protein; it can activate NF-kB signal pathway; It can significantly increase the expression of IFN gamma in memory CD4+ T cells, activate the immune system, and prolong the life cycle of tumor mice, so that it can be used in the treatment of tumors. The antibody of the present invention can be used in the preparation of drugs for treating tumors, autoimmune diseases and the like. The present invention has been completed on this basis.

### Terms

In the present invention, “VH-CDR1” and “CDR-H1” can be used interchangeably, both refer to the CDR1 of the variable region of the heavy chain; “VH-CDR2” and “CDR-H2” can be used interchangeably, both refer to the CDR2 of the variable region of the heavy chain; “VH-CDR3” and “CDR-H3” can be used interchangeably, both refer to the CDR3 of the variable region of the heavy chain. “VL-CDR1” and “CDR-L1” can be used interchangeably, both refer to the CDR1 of the variable region of the light chain; “VL-CDR2” and “CDR-L2” can be used interchangeably, both refer to the CDR2 of the variable region of the light chain; “VL-CDR3” and “CDR-L3” can be used interchangeably, both refer to the CDR3 of the variable region of the light chain.

Antibody

As used herein, the term “antibody” or “immunoglobulin” is a heterotetrameric glycoprotein of about 150,000 daltons with the same structural characteristics, which is composed of two identical light chains (L) and two identical heavy chains (H). Each light chain is connected to the heavy chain by a covalent disulfide bond, and the number of disulfide bonds between the heavy chains of different immunoglobulin isotypes is different. Each heavy and light chain also has regularly spaced intrachain disulfide bonds. Each heavy chain has a variable region (VH) at one end, followed by multiple constant regions. Each light chain has a variable region (VL) at one end and a constant region at the other end; the constant region of the light chain is opposite to the first constant region of the heavy chain, and the variable region of the light chain is opposite to the variable region of the heavy chain. Special amino acid residues form an interface between the variable regions of the light and heavy chains.

As used herein, the term “variable” means that certain parts of the variable region of the antibody are different in sequence, which forms the binding and specificity of various specific antibodies to their specific antigens. However, the variability is not evenly distributed throughout the variable regions of antibodies. It is concentrated in three fragments called complementarity determining regions (CDR) or hypervariable regions in the variable regions of the light and heavy chains. The more conserved part of the variable region is called the framework region (FR). The variable regions of the natural heavy chain and light chain each contain four FR regions, which are roughly in a β-folded configuration, connected by three CDRs forming a connecting loop, and in some cases can form a partial β-folded structure. The CDRs in each chain are closely joined together by the FR region and form the antigen binding site of the antibody together with the CDRs of the other chain (see Kabat et al., NIH Publ. No. 91-3242, Volume I, pages 647-669 (1991)). Constant regions do not directly participate in the binding of antibodies to antigens, but they exhibit different effector functions, such as participating in antibody-dependent cytotoxicity of antibodies.

The “light chains” of vertebrate antibodies (immunoglobulins) can be classified into one of two distinct categories (called κ and λ) based on the amino acid sequence of their constant regions. According to the amino acid sequence of the constant region of their heavy chains, immunoglobulins can be divided into different types. There are mainly five classes of immunoglobulins: IgA, IgD, IgE, IgG and IgM, some of which can be further divided into subclasses (isotypes), such as IgG1, IgG2, IgG3, IgG4, IgA and IgA2. The heavy chain constant regions corresponding to different classes of immunoglobulins are called α, δ, ε, γ, and μ, respectively. The subunit structures and three-dimensional configurations of different classes of immunoglobulins are well known to those skilled in the art.

Generally, the antigen-binding properties of antibodies can be described by 3 specific regions located in the variable regions of the heavy and light chains, called variable regions (CDR), which are divided into 4 framework regions (FR), the amino acid sequences of the 4 FRs are relatively conservative and do not directly participate in the binding reaction. These CDRs form a circular structure, and the β-sheet formed by the FRs in between are close to each other in space structure, and the CDRs on the heavy chain and the corresponding CDRs on the light chain constitute the antigen binding site of the antibody. The amino acid sequences of antibodies of the same type can be compared to determine which amino acids constitute the FR or CDR regions.

The invention includes not only complete antibodies, but also fragments of antibodies with immunological activity or fusion proteins formed by antibodies and other sequences. Therefore, the present invention also includes fragments, derivatives and analogues of the antibodies.

In the present invention, antibodies include murine, chimeric, humanized or fully human antibodies prepared by techniques well known to those skilled in the art. Recombinant antibodies, such as chimeric and humanized monoclonal antibodies, including human and non-human parts, can be obtained by standard DNA recombination techniques, and they are all useful antibodies. A chimeric antibody is a molecule in which different parts are derived from different animal species, for example, a chimeric antibody having a variable region from a mouse monoclonal antibody and a constant region from a human immunoglobulin (see, for example, U.S. Pat. Nos. 4,816,567 and 4,816,397, which is hereby incorporated by reference in its entirety). Humanized antibodies refer to antibody molecules derived from non-human species, with one or more complementarity determining regions (CDRs) derived from non-human species and framework regions derived from human immunoglobulin molecules (see U.S. Pat. No. 5,585,089, this article is hereby incorporated by reference in its entirety). These chimeric and humanized monoclonal antibodies can be prepared using DNA recombination techniques well known in the art.

In the present invention, antibodies can be monospecific, bispecific, trispecific, or more multispecific.

In the present invention, the antibody of the present invention also includes a conservative variant thereof, which means that compared with the amino acid sequence of the antibody of the present invention, there are at most 10, preferably at most 8, more preferably at most 5, and most preferably at most 3 amino acids are replaced by amino acids with similar or similar properties to form a polypeptide. These conservative variant polypeptides are best produced according to Table 1 through amino acid substitutions.

TABLE 1

| | | Preferred |
|---|---|---|
| Initial residue | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

Anti-OX40 Antibody

In the present invention, the antibody is an anti-OX40 antibody. The present invention provides a high-specificity and high-affinity antibody against OX40, which comprises a heavy chain and a light chain, the heavy chain contains a heavy chain variable region (VH) amino acid sequence, and the light chain contains a light chain variable region (VL) amino acid sequence.

Preferably,

The variable region of the heavy chain (VH) has a complementarity determining region CDR selected from the group consisting of:

The VH-CDR1 as shown in SEQ ID NO. 10n+3,

The VH-CDR2 as shown in SEQ ID NO. 10n+4, and

The VH-CDR3 as shown in SEQ ID NO. 10n+5;

wherein each n is independently 0, 1, 2, 3, 4, 5, 6, 7, 8 or 9;

The variable region of light chain (VL) has a complementarity determining region CDR selected from the group consisting of:

VL-CDR1 as shown in SEQ ID NO. 10n+8,

VL-CDR2 as shown in SEQ ID NO. 10n+9, and

VL-CDR3 as shown in SEQ ID NO. 10n+10;

wherein, each n is independently 0, 1, 2, 3, 4, 5, 6, 7, 8 or 9;

wherein, any one of the above amino acid sequences further includes a derivative sequence that is optionally added, deleted, modified and/or substituted for at least one amino acid and can retain the binding affinity of OX40.

Preferably, the heavy chain variable region (VH) includes the following three complementarity determining region CDRs:

The VH-CDR1 as shown in SEQ ID NO. 10n+3,

The VH-CDR2 as shown in SEQ ID NO. 10n+4, and

The VH-CDR3 as shown in SEQ ID NO. 10n+5;

The light chain variable region (VL) includes the following three complementarity determining region CDRs:

VL-CDR1 as shown in SEQ ID NO. 10n+8,

VL-CDR2 as shown in SEQ ID NO. 10n+9, and

VL-CDR3 as shown in SEQ ID NO. 10n+10;

Each n is independently 0, 1 or 2; preferably n is 0 or 1; Wherein, any one of the above amino acid sequences further includes a derivative sequence that is optionally added, deleted, modified and/or substituted for at least one amino acid and can retain the binding affinity of OX40.

In another preferred embodiment, the sequence formed by adding, deleting, modifying and/or substituting at least one amino acid sequence is preferably an amino acid sequence with homology or sequence identity of at least 80%, preferably at least 85%, more preferably, at least 90%, and most preferably at least 95%.

Methods of determining sequence homology or identity well-known to those of ordinary skill in the art include, but are not limited to: Computational Molecular Biology, Lesk, edited by A. M., Oxford University Press, New York, 1988; Biocomputing: Information Biocomputing: Informatics and Genome Projects, Smith, edited by D. W., Academic Press, New York, 1993; Computer Analysis of Sequence Data, Part 1, Griffin, A. M. and Griffin, edited by H. G., Humana Press, New Jersey, 1994; Sequence Analysis in Molecular Biology, von Heinje, G., Academic Press, 1987 and Sequence Analysis Primer, Gribskov, M. and Devereux, J. Ed. M Stockton Press, New York, 1991 and Carillo, H. and Lipman, D., SIAM J. Applied Math., 48:1073 (1988). The preferred method for determining identity is to obtain the largest match between the tested sequences. The method for determining identity is compiled in a publicly available computer program. Preferred computer program methods for determining the identity between two sequences include but are not limited to: GCG package (Devereux, J. et al., 1984), BLASTP, BLASTN and FASTA (Altschul, S, F. et al., 1990). The public can obtain the BLASTX program from NCBI and other sources (BLAST Handbook, Altschul, S. et al., NCBI NLM NIH Bethesda, Md. 20894; Altschul, S. et al., 1990). The well-known Smith Waterman algorithm can also be used to determine identity.

Preferably, the antibody as described herein is one or more of an antibody full-length protein, an antigen-antibody binding domain protein fragment, a bispecific antibody, a multispecific antibody, a single chain antibody (Single chain antibody fragment, ScFv), single domain antibody (SdAb) and Single-domain antibody, and monoclonal or polyclonal antibodies prepared from the above antibodies. The monoclonal antibody can be developed by a variety of approaches and technologies, including hybridoma technology, phage display technology, single lymphocyte gene cloning technology, etc. The mainstream is to prepare monoclonal antibodies from wild-type or transgenic mice through hybridoma technology.

The antibody full-length protein is a conventional antibody full-length protein in the art, which comprises a heavy chain variable region, a light chain variable region, a heavy chain constant region and a light chain constant region. The heavy chain variable region and light chain variable region of the protein, the human heavy chain constant region and the human light chain constant region constitute a fully human antibody full-length protein. Preferably, the antibody full-length protein is IgG1, IgG2, IgG3 or IgG4. The antibody of the present invention may be a double-chain or single-chain antibody, and may be selected from the group consisting of animal-derived antibodies, chimeric antibodies, humanized antibodies, more preferably humanized antibodies, human-animal chimeric antibodies, more preferably a fully humanized antibody.

The antibody derivatives of the present invention may be single-chain antibodies, and/or antibody fragments, such as: Fab, Fab′, (Fab′)2 or other known antibody derivatives in the field and the like, and any one or more of IgA, IgD, IgE, IgG and IgM antibodies or antibodies of other subtypes.

The single chain antibody is a conventional single chain antibody in the art, which comprises a heavy chain variable region, a light chain variable region and a short peptide of 15-20 amino acids.

Wherein the animal is preferably a mammal, such as a mouse.

The antibody of the present invention may be a chimeric antibody, a humanized antibody, a CDR grafted and/or modified antibody targeting OX40 (such as human OX40)

In the above content of the present invention, the number of added, deleted, modified and/or substituted amino acids is preferably no more than 40% of the total number of amino acids in the initial amino acid sequence, more preferably no more than 35%, more preferably 1-33%, more preferably 5-30%, more preferably 10-25%, more preferably 15-20%.

In the above content of the present invention, more preferably, the number of added, deleted, modified and/or substituted amino acids can be 1-7, more preferably 1-5, more preferably 1-3, more preferably 1-2.

In another preferred example, the heavy chain variable region of the antibody comprises the amino acid sequence as shown in SEQ ID NO. 101, 1, 11 or 21.

In another preferred example, the light chain variable region of the antibody contains the amino acid sequence as shown in SEQ ID NO. 103, 6, 16 or 26.

In another preferred example, the amino acid sequence of the variable region of the heavy chain (VH) of the antibody targeting OX40, and/or the amino acid sequence of the variable region of the light chain are shown in Table 2 below:

TABLE 2

| Antibody | VH sequence | VL sequence |
|---|---|---|
| number | Number | Number |
| 1 | 101 | 103 |
| 2 | 1 | 6 |
| 3 | 11 | 16 |
| 4 | 21 | 26 |

In another preferred example, the antibodies targeting OX40 are Humab035-24, mab035, mab053, mab041, mab058, mab034, mab049, mab064, mab033, mab047, and mab055.

In another preferred example, the antibodies targeting OX40 are Humab035-24, Mab035, Mab053, Mab041.

In another preferred example, the antibody targeting OX40 is Humab035-24.

In another preferred example, the antibody targeting OX40 is mab035.

In another preferred example, the antibody targeting OX40 is mab053.

In another preferred example, the antibody targeting OX40 is mab041.

Recombinant Protein

The invention also provides a recombinant protein comprising one or more of the heavy chain CDR1 (VH-CDR1), the heavy chain CDR2 (VH-CDR2) and the heavy chain CDR3 (VH-CDR3) of the OX40 antibody, and/or one or more of the light chain CDR1 (VL-CDR1), light chain CDR2 (VL-CDR2) and light chain CDR3 (VL-CDR3) of the OX40 antibody,

the sequence of the heavy chain CDR1-3 is as follows:

The VH-CDR1 as shown in SEQ ID NO. 10n+3,

The VH-CDR2 as shown in SEQ ID NO. 10n+4, and

The VH-CDR3 as shown in SEQ ID NO. 10n+5;

the sequence of the light chain CDR1-3 is as follows:

VL-CDR1 as shown in SEQ ID NO. 10n+8,

VL-CDR2 as shown in SEQ ID NO. 10n+9, and

VL-CDR3 as shown in SEQ ID NO. 10n+10;

each n is independently 0, 1, 2, 3, 4, 5, 6, 7, 8 or 9; preferably n is 0 or 1;

wherein, any one of the above amino acid sequences further includes a derivative sequence that is optionally added, deleted, modified and/or substituted for at least one amino acid and can retain the binding affinity of OX40.

In another preferred embodiment, the sequence formed by adding, deleting, modifying and/or substituting at least one amino acid sequence is preferably an amino acid sequence with homology or sequence identity of at least 80%, preferably at least 85%, more preferably, at least 90%, and most preferably at least 95%.

In another preferred embodiment, the recombinant protein of the present invention includes the heavy chain variable region of the OX40 antibody and/or the light chain variable region of the OX40 antibody. The heavy chain variable region of the antibody contains the amino acid sequence as shown in SEQ ID NO. 101, 1, 11 or 21; the light chain variable region of the antibody contains the amino acid sequence as shown in SEQ ID NO. 103, 6, 16 or 26.

In another preferred embodiment, the recombinant protein of the present invention includes the heavy chain variable region of the OX40 antibody and the light chain variable region of the OX40 antibody. The heavy chain variable region has the amino acid sequence as shown in SEQ ID NO. 101 or 10n+1, the light chain variable region has the amino acid sequence as shown in SEQ ID NO. 103 or 10n+6, wherein n is 0, 1, 2, 3, 4, 5, 6, 7, 8, or 9.

In another preferred embodiment, the recombinant protein of the present invention includes the heavy chain variable region of the OX40 antibody and the light chain variable region of the OX40 antibody. The heavy chain variable region of the antibody contains the amino acid sequence as shown in SEQ ID NO. 101, 1, 11, or 21, and the light chain variable region of the antibody contains the amino acid sequence as shown in SEQ ID NO. 103, 6, 16 or 26.

In another preferred embodiment, the sequence numberings of the recombinant protein and the amino acid sequences of the heavy chain CDR1-3 and the light chain CDR1-3 included in the recombinant protein are shown in Table 3:

TABLE 3
Sequence numbering of the amino acid sequence
of heavy chain CDR1-3 and light chain CDR1-3

| Recombinant | Heavy chain protein | | | | Light chain protein | | | |
|---|---|---|---|---|---|---|---|---|
| protein | Variable | VH- | VH- | VH- | Variable | VL- | VL- | VL- |
| number | region | CDR1 | CDR2 | CDR3 | region | CDR1 | CDR2 | CDR3 |
| 1 | 101 | 3 | 4 | 5 | 103 | 8 | 9 | 10 |
| 2 | 1 | 3 | 4 | 5 | 6 | 8 | 9 | 10 |
| 3 | 11 | 13 | 14 | 15 | 16 | 18 | 19 | 20 |
| 4 | 21 | 23 | 24 | 25 | 26 | 28 | 29 | 30 |

Wherein, any one of the above amino acid sequences further includes a derivative sequence that is optionally added, deleted, modified and/or substituted for at least one amino acid and can retain the binding affinity of OX40.

Preferably, the recombinant protein further comprises an antibody heavy chain constant region and/or an antibody light chain constant region, the antibody heavy chain constant region is conventional in the art, preferably a rat-derived antibody heavy chain constant region or a human-derived antibody heavy chain constant region, more preferably a human-derived antibody heavy chain constant region. The antibody light chain constant region is conventional in the art, preferably a rat-derived light chain antibody constant region or a human-derived antibody light chain constant region, and more preferably a human-derived antibody light chain constant region.

The recombinant protein is a conventional protein in the art, preferably one or more of an antibody full-length protein, an antigen-antibody binding domain protein fragment, a bispecific antibody, a multispecific antibody, a single chain antibody (single chain antibody fragment, scFv), single domain antibody (SdAb) and single-domain antibody, and monoclonal or polyclonal antibodies prepared from the above antibodies. The monoclonal antibody can be developed by a variety of approaches and technologies, including hybridoma technology, phage display technology, single lymphocyte gene cloning technology, etc. The mainstream is to prepare monoclonal antibodies from wild-type or transgenic mice through hybridoma technology.

The antibody full-length protein is a conventional antibody full-length protein in the art, which comprises a heavy chain variable region, a light chain variable region, a heavy chain constant region and a light chain constant region. The heavy chain variable region and light chain variable region of the protein, the human heavy chain constant region and the human light chain constant region constitute the full-length protein of the fully human antibody. Preferably, the antibody full-length protein is IgG1, IgG2, IgG3 or IgG4.

The single chain antibody is a conventional single chain antibody in the art, which comprises a heavy chain variable region, a light chain variable region and a short peptide of 15-20 amino acids.

The antigen-antibody binding domain protein fragment is a conventional antigen-antibody binding domain protein fragment in the art, which includes a light chain variable region, a Fd segment of a light chain constant region and heavy chain constant region. Preferably, the antigen-antibody binding domain protein fragment is Fab and F(Ab′).

The single domain antibody is a conventional single domain antibody in the art, which includes a heavy chain variable region and a heavy chain constant region.

The single-region antibody is a conventional single-region antibody in the art, which only includes the heavy chain variable region.

The preparation method of the recombinant protein is the conventional preparation method in the field. The preparation method is preferably: It is isolated and obtained from the expression transformant that recombinantly expresses the protein or obtained by artificially synthesizing the protein sequence. The method of separating and obtaining from the expression transformant that recombinantly express the protein is preferably as follows: cloning the nucleic acid molecule encoding the protein and carrying the point mutation into a recombinant vector, and transforming the obtained recombinant vector into the transformant to obtain the recombinant expression transformant and the recombinant protein can be obtained by separation and purification by culturing the obtained recombinant expression transformant.

Nucleic Acid

The present invention also provides a nucleic acid encoding the above-mentioned antibody (e.g., anti-OX40 antibody) or recombinant protein or the variable region of the heavy chain or the variable region of the light chain of an anti-OX40 antibody.

Preferably, the nucleotide sequence of the nucleic acid encoding the heavy chain variable region is as shown in the sequence listing of SEQ ID NO. 102, 2, 12, 22, 32, 42, 52, 62, 72, 82 or 92; and/or, the nucleotide sequence of the nucleic acid encoding the light chain variable region is as shown in the sequence listing of SEQ ID NO. 104, 7, 17, 27, 37, 47, 57, 67, 77, 87 or 97.

Preferably, the nucleotide sequence of the nucleic acid encoding the heavy chain variable region is as shown in the sequence listing of SEQ ID NO. 102, 2, 12 or 22; and the nucleotide sequence of the nucleic acid encoding the light chain variable region is as shown in the sequence listing of SEQ ID NO. 104, 7, 17 or 27.

The preparation method of the nucleic acid is a conventional preparation method in the field, and preferably comprises the following steps: obtaining a nucleic acid molecule encoding the above protein by a gene cloning technology, or obtaining a nucleic acid molecule encoding the above protein by an artificial full sequence synthesis method.

Those skilled in the art know that the base sequence encoding the amino acid sequence of the above-mentioned protein can be replaced, deleted, changed, inserted or added as appropriate to provide a polynucleotide homolog. The polynucleotide homologs of the present invention can be prepared by replacing, deleting or adding one or more bases of the gene encoding the protein sequence within the scope of maintaining antibody activity.

Vector

The invention also provides a recombinant expression vector comprising the nucleic acid.

Wherein the recombinant expression vector can be obtained by conventional methods in the art, that is, the nucleic acid molecule of the present invention is connected to various expression vectors to be constructed. The expression vector is a variety of vectors conventional in the art, as long as it can accommodate the aforementioned nucleic acid molecules. The vectors preferably include various plasmids, cosmids, bacteriophages or viral vectors.

The invention also provides a recombinant expression transformant comprising the above-mentioned recombinant expression vector.

Wherein the preparation method of the recombinant expression transformant is the conventional preparation method in the field, preferably: It is prepared by transforming the above-mentioned recombinant expression vector into a host cell. The host cell is a variety of conventional host cells in the art, as long as the recombinant expression vector can replicate itself stably and the nucleic acid carried can be effectively expressed. Preferably, the host cell is E. coli TG1 or E. coli BL21 cell (expressing single-chain antibody or Fab antibody), or HEK293 or CHO cell (expressing full-length IgG antibody). The aforementioned recombinant expression plasmid is transformed into a host cell to obtain the preferred recombinant expression transformant of the present invention. The transformation method is a conventional transformation method in the field, preferably a chemical transformation method, a heat shock method or an electrotransformation method.

Antibody Preparation

The sequence of the DNA molecule of the antibody or its fragment of the present invention can be obtained by conventional techniques, such as PCR amplification or genomic library screening. In addition, the coding sequences of the light chain and the heavy chain can also be fused together to form a single chain antibody.

Once the relevant sequences are obtained, the relevant sequences can be obtained in large quantities by recombination method. It is usually cloned into a vector, then transferred into a cell, and then the relevant sequence is isolated from the host cell after proliferation by conventional methods.

In addition, the relevant sequences can also be synthesized by artificial synthesis, especially when the fragment length is short. Usually, by first synthesizing multiple small fragments, and then ligating to obtain very long fragments.

At present, the DNA sequence encoding the antibody (or fragment or derivative thereof) of the present invention can be obtained completely through chemical synthesis. The DNA sequence can then be introduced into various existing DNA molecules (or such as vectors) and cells known in the art. In addition, mutations can also be introduced into the protein sequence of the present invention through chemical synthesis.

The present invention also relates to a vector containing the above-mentioned appropriate DNA sequence and an appropriate promoter or control sequence. These vectors can be used to transform appropriate host cells so that they can express proteins.

The host cell may be a prokaryotic cell, such as a bacterial cell; or lower eukaryotic cells, such as yeast cells; or higher eukaryotic cells, such as mammalian cells. Preferred animal cells include (but are not limited to): CHO-S, HEK-293 cells.

Generally, the transformed host cell is cultured under conditions suitable for expression of the antibody of the present invention. Then the antibody of the present invention is purified by conventional immunoglobulin purification steps, such as protein A-Sepharose, hydroxyapatite chromatography, gel electrophoresis, dialysis, ion exchange chromatography, hydrophobic chromatography, molecular sieve chromatography or affinity chromatography and other conventional separation and purification methods well known to those skilled in the art.

The obtained monoclonal antibody can be identified by conventional means. For example, the binding specificity of monoclonal antibodies can be determined by immunoprecipitation or in vitro binding assays (such as radioimmunoassay (RIA) or enzyme-linked immunosorbent assay (ELISA)). The binding affinity of the monoclonal antibody can be determined, for example, by the Scatchard analysis of Munson et al., Anal. Biochem., 107:220 (1980).

The antibody of the present invention can be expressed in the cell, on the cell membrane, or secreted out of the cell. If necessary, the physical, chemical, and other characteristics can be used to separate and purify the recombinant protein through various separation methods. These methods are well known to those skilled in the art. Examples of these methods include, but are not limited to: conventional renaturation treatment, treatment with protein precipitation agent (salting out method), centrifugation, bacteria broken through osmosis, ultrasonic treatment, ultracentrifugation, molecular sieve chromatography (gel filtration), adsorption chromatography, ion exchange chromatography, high performance liquid chromatography (HPLC) and various other liquid chromatography techniques and combinations of these methods.

Antibody-Drug Conjugate (ADC)

The present invention also provides antibody-drug conjugate (ADC) based on the antibody of the present invention.

Typically, the antibody-drug conjugate comprises the antibody and an effector molecule, the antibody being coupled to the effector molecule, and preferably chemically coupled. Wherein the effector molecule is preferably a drug having therapeutic activity. In addition, the effector molecule may be one or more of toxic proteins, chemotherapeutic drugs, small molecule drugs or radionuclides.

The antibody of the present invention can be coupled with the effector molecule by a coupling agent. Examples of the coupling agent may be any one or more of a non-selective coupling agent, a coupling agent using a carboxyl group, a peptide chain, and a coupling agent using a disulfide bond. The non-selective coupling agent refers to a compound that makes the effector molecule and the antibody form a covalent bond, such as glutaraldehyde. The coupling agent using carboxyl groups can be any one or more of cis-aconitic acid anhydride coupling agents (such as cis-aconitic acid anhydride) and acyl hydrazone coupling agents (coupling sites are acyl hydrazones).

Certain residues on the antibody (such as Cys or Lys, etc.) are used to connect to a variety of functional groups, including imaging reagents (such as chromophores and fluorescent groups), diagnostic reagents (such as MRI contrast agents and radioisotopes), stabilizers (such as glycol polymers) and therapeutic agents. The antibody can be conjugated to the functional agent to form an antibody-functional agent conjugate. Functional agents (e.g., drugs, detection reagents, stabilizers) are coupled (covalently linked) to the antibody. The functional agent may be directly or indirectly linked to the antibody through a linker.

Antibodies can be conjugated with drugs to form antibody-drug conjugates (ADCs). Typically, the ADC contains a linker between the drug and the antibody. The linker can be a degradable or a non-degradable linker. Degradable linkers are typically easily degraded in the intracellular environment, for example, the linker is degraded at the target site, so that the drug is released from the antibody. Suitable degradable linkers include, for example, enzymatically degraded linkers, including peptidyl-containing linkers that can be degraded by intracellular proteases (such as lysosomal proteases or endosomal proteases), or sugar linkers, for example, a glucuronide-containing linker that can be degraded by glucuronidase. The peptidyl linker may include, for example, dipeptides such as valine-citrulline, phenylalanine-lysine or valine-alanine. Other suitable degradable linkers include, for example, pH-sensitive linkers (for example, linkers that are hydrolyzed at a pH of less than 5.5, such as hydrazone linkers) and linkers that degrade under reducing conditions (for example, disulfide bond linkers). Non-degradable linkers typically release the drug under conditions where the antibody is hydrolyzed by a protease.

Before being connected to the antibody, the linker has a reactive group capable of reacting with certain amino acid residues, and the connection is achieved through the reactive group. Sulfhydryl-specific reactive groups are preferred and include, for example, maleimide compounds, halogenated amides (such as iodine, bromine, or chloro); halogenated esters (such as iodine, bromine, or chloro); halogenated methyl ketones (such as iodine, bromine or chloro), benzyl halides (such as iodine, bromine or chloro); vinyl sulfone, pyridyl disulfide; mercury derivatives such as 3,6-Di-(mercury methyl) dioxane, and the counter ion is acetate, chloride or nitrate; and polymethylene dimethyl sulfide thiosulfonate. The linker may include, for example, maleimide linked to the antibody via thiosuccinimide.

The drug can be any cytotoxic, inhibiting cell growth or immunosuppressive drug. In embodiments, the linker connects the antibody and the drug, and the drug has a functional group that can be bonded to the linker. For example, the drug may have an amino group, a carboxyl group, a sulfhydryl group, a hydroxyl group, or a ketone group that can form a bond with the linker. In the case where the drug is directly connected to the linker, the drug has a reactive active group before being connected to the antibody.

Useful drug categories include, for example, anti-tubulin drugs, DNA minor groove binding reagents, DNA replication inhibitors, alkylating reagents, antibiotics, folate antagonists, antimetabolites, chemotherapy sensitizers, topoisomerase inhibitors, Vinca Alkaloids, etc. Examples of particularly useful cytotoxic drugs include, for example, DNA minor groove binding reagents, DNA alkylating reagents, and tubulin inhibitors. Typical cytotoxic drugs include, for example, auristatins, camptothecins, duocarmycins, etoposides, maytansines and maytansinoids (e.g., DM1 and DM4), taxanes, benzodiazepines or benzodiazepine containing drugs (e.g., pyrrolo[1,4] benzodiazepines (PBDs), indolinobenzodiazepines and oxazolidinobenzodiazepines and vinca alkaloids.

In the present invention, the drug-linker can be used to form ADC in one simple step. In other embodiments, bifunctional linker compounds can be used to form ADCs in a two-step or multi-step process. For example, the cysteine residue reacts with the reactive part of the linker in the first step, and in the subsequent step, the functional group on the linker reacts with the drug to form ADC.

Generally, the functional group on the linker is selected to facilitate the specific reaction with the appropriate reactive group on the drug moiety. As a non-limiting example, the azide-based moiety can be used to specifically react with the reactive alkynyl group on the drug moiety. The drug is covalently bound to the linker through the 1,3-dipolar cycloaddition between the azide and alkynyl groups. Other useful functional groups include, for example, ketones and aldehydes (suitable for reacting with hydrazides and alkoxyamines), phosphines (suitable for reacting with azides); isocyanates and isothiocyanates (suitable for reaction with amines and alcohols); and activated esters, such as N-hydroxysuccinimide ester (suitable for reaction with amines and alcohols). These and other ligation strategies, such as those described in “Bioconjugation Technology”, Second Edition (Elsevier), are well known to those skilled in the art. Those skilled in the art can understand that for the selective reaction between the drug moiety and the linker, when a complementary pair of reactive functional groups is selected, each member of the complementary pair can be used for both linkers and drugs.

The present invention also provides a method for preparing ADC, which may further include: combining an antibody with a drug-linker compound under conditions sufficient to form an antibody conjugate (ADC).

In certain embodiments, the methods of the invention include combining the antibody with a bifunctional linker compound under conditions sufficient to form an antibody-linker conjugate. In these embodiments, the method of the present invention further includes: binding the antibody linker conjugate to the drug moiety under conditions sufficient to covalently link the drug moiety to the antibody via a linker.

In some embodiment, the antibody-drug conjugate ADC is shown in the following molecular formula:

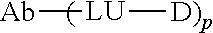

wherein

Ab is an antibody,

LU is a linker;

D is a drug;

and the subscript p is a value selected from 1 to 8.

Application

The present invention also provides the use of the antibody, antibody conjugate ADC, recombinant protein, and/or immune cell of the present invention, for example, for preparing diagnostic preparations or preparing medicines.

Preferably, the drug is a drug for preventing and/or treating diseases related to abnormal expression or function of OX40.

In the present invention, the diseases related to abnormal expression or function of OX40 are diseases related to abnormal expression or function of OX40 conventionally in the art. Preferably, the disease related to the abnormal expression or function of OX40 is cancer or autoimmune disease.

In the present invention, the cancer is a conventional cancer in the field, preferably breast cancer, melanoma, B-cell lymphoma, head and neck cancer, colon cancer, prostate cancer, and lung cancer. In the present invention, the autoimmune disease is a conventional autoimmune disease in the art, preferably rheumatoid arthritis, inflammatory hyperproliferative skin disease, multiple sclerosis, inflammatory bowel disease, asthma, autoimmune myocardioptis, systemic lupus erythematosus.

The uses of the antibodies, ADCs, recombinant proteins, and/or immune cells of the present invention include (but are not limited to):

(i) diagnosis, prevention and/or treatment of tumor occurrence, growth and/or metastasis, especially tumors with high OX40 expression. The tumors include (but are not limited to): breast cancer, melanoma, B-cell lymphoma, head and neck cancer, colon cancer, prostate cancer, lung cancer.

(ii) diagnosis, prevention and/or treatment of autoimmune diseases, including (but are not limited to): rheumatoid arthritis, inflammatory hyperproliferative skin disease, multiple sclerosis, inflammatory bowel disease, asthma, autoimmune myocardioptis, systemic lupus erythematosus.

Detection Purposes and Kits

The antibody or ADC of the present invention can be used in detection applications, for example, to detect samples, so as to provide diagnostic information.

In the present invention, the samples (specimen) used include cells, tissue samples and biopsy specimens. The term “biopsy” used in the present invention shall include all kinds of biopsy known to those skilled in the art. Therefore, the biopsy used in the present invention may include, for example, excision samples of tumors, tissue samples prepared by endoscopic methods or organ puncture or needle biopsy.

The samples used in the present invention include fixed or preserved cell or tissue samples.

The present invention also provides a kit containing the antibody (or fragment thereof) of the present invention. In a preferred embodiment of the present invention, the kit further includes a container, instructions for use, buffers, and the like. In a preferred embodiment, the antibody of the present invention can be immobilized on a detection plate.

Pharmaceutical Composition

The invention also provides a composition. In a preferred example, the composition is a pharmaceutical composition, which contains the above-mentioned antibody or active fragment or fusion protein or ADC thereof or corresponding immune cell, and a pharmaceutically acceptable carrier. Generally, these substances can be formulated in a non-toxic, inert and pharmaceutically acceptable aqueous carrier medium, wherein the pH is usually about 5-8, preferably about 6-8, although the pH value can vary with the nature of the substance being formulated and the condition to be treated.

The formulated pharmaceutical composition can be administered by conventional routes, including (but are not limited to): intratumoral, intraperitoneal, intravenous, or topical administration. Typically, the route of administration of the pharmaceutical composition of the present invention is preferably injection or oral administration. The injection administration preferably includes intravenous injection, intramuscular injection, intraperitoneal injection, intradermal injection, or subcutaneous injection. The pharmaceutical composition is a variety of conventional dosage forms in the art, preferably in the form of solid, semi-solid or liquid, and can be an aqueous solution, non-aqueous solution or suspension, and more preferably a tablet, capsule, or granule, injection or infusion, etc.

The antibody of the present invention can also be used for cell therapy by expressing the nucleotide sequence in a cell, for example, the antibody is used for chimeric antigen receptor T cell immunotherapy (CAR-T) and the like.

The pharmaceutical composition of the present invention is a pharmaceutical composition for preventing and/or treating diseases related to abnormal expression or function of OX40.

The pharmaceutical composition of the present invention can be directly used to bind OX40 protein molecules, and thus can be used to prevent and treat tumors and other diseases.

The pharmaceutical composition of the present invention contains a safe and effective amount (such as 0.001-99 wt %, preferably 0.01-90 wt %, more preferably 0.1-80 wt %) of the above-mentioned monoclonal antibody (or conjugate thereof) of the present invention and a pharmaceutical acceptable carrier or excipient. Such carriers include (but are not limited to): saline, buffer, glucose, water, glycerol, ethanol, and combinations thereof. The pharmaceutical preparation should match the mode of administration. The pharmaceutical composition of the present invention can be prepared in the form of injections, for example, it can be prepared by conventional methods with physiological saline or an aqueous solution containing glucose and other adjuvants. Pharmaceutical compositions such as injections and solutions should be manufactured under aseptic conditions. The dosage of the active ingredient is a therapeutically effective amount, for example, about 1 microgram/kg body weight to about 5 mg/kg body weight per day. In addition, the polypeptides of the present invention can also be used together with other therapeutic agents.

In the present invention, preferably, the pharmaceutical composition of the present invention further includes one or more pharmaceutical carriers. The pharmaceutical carrier is a conventional pharmaceutical carrier in the art, and the pharmaceutical carrier can be any suitable physiologically or pharmaceutically acceptable pharmaceutical excipient. The pharmaceutical excipients are conventional pharmaceutical excipients in the field, and preferably include pharmaceutically acceptable excipients, fillers or diluents. More preferably, the pharmaceutical composition includes 0.01-99.99% of the aforementioned protein and 0.01-99.99% of a pharmaceutical carrier, and the percentage is a mass percentage of the pharmaceutical composition.

In the present invention, preferably, the administration amount of the pharmaceutical composition is an effective amount, and the effective amount is an amount capable of alleviating or delaying the progression of the disease, degenerative or traumatic condition. The effective amount can be determined on an individual basis and will be partly based on consideration of the symptoms to be treated and the results sought. Those skilled in the art can determine the effective amount by using the aforementioned factors such as individual basis and using no more than conventional experiments.

When using the pharmaceutical composition, a safe and effective amount of the immunoconjugate is administered to the mammal, wherein the safe and effective amount is usually at least about 10 micrograms/kg body weight, and in most cases, it does not exceed about 50 mg/kg body weight, preferably the dosage is about 10 micrograms/kg body weight to about 20 mg/kg body weight. Of course, the specific dosage should also consider factors such as the route of administration, the patient's health status, etc., which are within the skill range of a skilled physician.

The present invention provides the application of the above-mentioned pharmaceutical composition in the preparation of drugs for preventing and/or treating diseases related to abnormal expression or function of OX40. Preferably, the disease related to the abnormal expression or function of OX40 is cancer or autoimmune disease.

Method and Composition for Detecting OX40 Protein in Sample

The present invention also provides a method for detecting OX40 protein in a sample (for example, detecting over-expressing OX40 cells), which includes the following steps: the above-mentioned antibody is contacted with the sample to be tested in vitro, and it is sufficient to detect whether the above-mentioned antibody binds to the sample to be tested to form an antigen-antibody complex.

The meaning of overexpression is conventional in the art, and refers to the overexpression of OX40 protein in RNA or protein in the sample to be tested (due to increased transcription, post-transcriptional processing, translation, post-translational processing and changes in protein degradation), as well as local overexpression and increased functional activity due to changes in protein transport mode (increased nuclear localization) (such as in the case of increased enzymatic hydrolysis of the substrate).

In the present invention, the detection method of whether the above-mentioned binding to form an antigen-antibody complex is a conventional detection method in the art, preferably a flow cytometry (FACS) detection.

The present invention provides a composition for detecting OX40 protein in a sample, which includes the above-mentioned antibody, recombinant protein, antibody conjugate, immune cell, or a combination thereof as an active ingredient. Preferably, it also includes a compound composed of the above-mentioned functional fragments of the antibody as an active ingredient.

On the basis of complying with common knowledge in the art, the above preferred conditions can be arbitrarily combined to obtain preferred examples of the present invention.

The main advantages of the present invention are:

(1) The protein of the present invention is the OX40 antibody, which has high affinity with human-derived OX40 protein, and has similar binding ability with monkey-derived OX40 protein, can activate the NF-kB signaling pathway, and can significantly increase the expression of IFN gamma in memory CD4+ T cells.

(2) Some of the OX40 antibodies of the present invention have different epitopes compared with the antibodies that have been developed so far.

(3) Some of the OX40 antibodies of the present invention can better activate the NF-kB signaling pathway and the function of T cells, and prolong the life cycle of MC38 tumor mice compared with the antibodies that have been developed so far.

The present invention will be further elaborated below in conjunction with specific embodiments. It should be understood that these embodiments are only used to illustrate the present invention and not to limit the scope of the present invention. The following examples do not specify the detailed conditions of the experimental methods, usually according to the conventional conditions such as the conditions described in the “Molecular Cloning Laboratory Guide” (translated by Huang Peitang et al., Beijing: Science Press, 2002) by Sambrook. J et al., Or in accordance with the conditions recommended by the manufacturer (such as product specifications). Unless otherwise stated, percentages and parts are calculated by weight. The experimental materials and reagents used in the following examples can be obtained from commercial channels unless otherwise specified.

The room temperature described in the embodiment is a conventional room temperature in the art, and is generally 10-30° C.

Unless otherwise specified, the PBS described in the examples is PBS phosphate buffer, pH 7.4.

### Example 1 Preparation of OX40 Antibody

1.1 Preparation of immunogen A

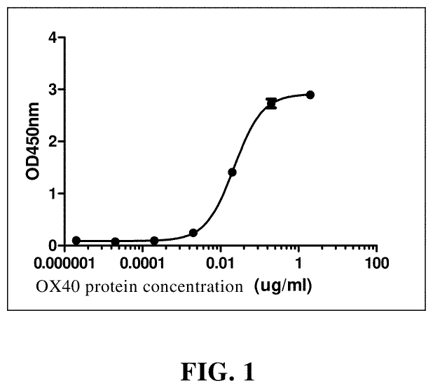

The nucleotide sequence containing the amino acid sequence Leu29-Ala216 (human-derived OX40 protein sequence from Uniprot database, protein sequence number P43489) encoding the extracellular region of human-derived OX40 protein was cloned into pTT5 vector with human IgG Fc fragment (hFc) and prepared plasmids according to established standard molecular biology methods. For specific methods, see Sambrook, J., Fritsch, E F, and Maniatis, T. (1989). Molecular Cloning: A Laboratory Manual, Second Edition (Plainview, N.Y.: Cold Spring Harbor Laboratory Press). Transfecting HEK293 cells with the prepared plasmid transiently (polyetherimide (PEI), purchased from Polysciences) and expanding the culture. After 4 days, the cell culture fluid was collected, and the cell components were removed by centrifugation to obtain the culture supernatant containing the extracellular region of OX40 protein. The culture supernatant was loaded onto a protein A affinity chromatography column (purchased from GE Healthcare), and at the same time, monitoring the change of UV absorption value (A280 nm) with UV detector. After loading the sample, washing the protein A affinity chromatography column with PBS until the UV absorption value returns to the baseline, and then eluted with 0.1M glycine hydrochloric acid (pH 2.5) to collect the extracellular domain of OX40 protein with hFc tag (OX40 ECD-hFc) eluted from the protein A affinity chromatography column, and dialyzed overnight in a refrigerator at 4° C. with PBS. The dialyzed protein was sterile filtered by 0.22 μm and stored at −80° C. to obtain purified immunogen A. Immunogen A undergoes a series of quality control tests before use, such as testing its protein concentration, purity, molecular weight, biological activity, etc. The results are shown in FIG. 1. FIG. 1 illustrates that the binding of OX40 and OX40L at the protein level varies with the concentration of OX40. The data in the figure is the OD450 nm value.

Among them, the biological activity of immunogen A is detected by ELISA, specifically:

Diluting OX40L recombinant protein (purchased from R&D) with PBS to 1 μg/mL, adding 100W/well to the ELISA microplate, incubated overnight at 4° C.; using ELISA blocking solution (containing 1% BSA, pH7.4 of PBS phosphate buffer, the percentage is the mass percentage.) After sealing at 37° C. for two hours, adding OX40 ECD-hFc (i.e., immunogen A) in gradient dilutions, and incubated at 37° C. for 1 hour; adding horseradish peroxidase labeled goat anti-human IgG (purchased from Sigma, A8667), after 30 minutes of incubation at 37° C.; adding 100 μL/well of TMB color developing solution (purchased from InnoReagents, EL0009), after 15 minutes of incubation at room temperature, adding 50 μL of 1N hydrochloric acid to terminate the chromogenic reaction. Reading the OD450 nm value with an ELISA plate reader.

1.2 Preparation of Immunogen B

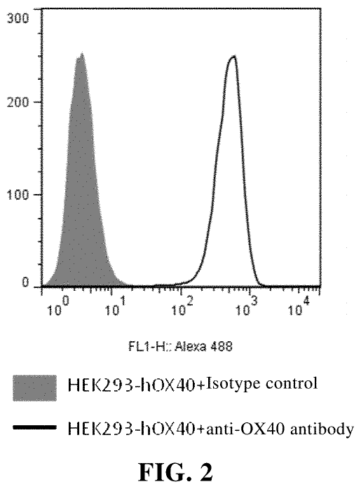

The nucleotide sequence encoding the full-length amino acid sequence of human-derived (human) OX40 (the sequence is from the Uniprot database, the protein sequence number is P43489) was cloned into the pLVX-IRES-Puro vector and the plasmid (herein referred to as pLVX-hOX40-IRES-Puro) was prepared. After plasmid transfection of HEK293 cell line, subcloning in 96-well culture plate by limiting dilution method, it was selectively cultured in DMEM medium containing 10% fetal bovine serum containing 0.5 μg/ml puromycin. After about 2 weeks of culture, some monoclonal wells were selected and expanded into a 24-well plate, and then expanded into a 6-well plate after about 3-4 days. The amplified clones were screened by flow cytometry (FACS) with OX40 antibody (purchased from R&D). Choosing a cell line with better growth, higher fluorescence intensity, and monoclonal cell line to continue to expand culture and freeze in liquid nitrogen to obtain immunogen B (HEK293-hOX40 cells). The FACS result is shown in FIG. 2. FIG. 2 shows that the HEK293 cell line stably expressing OX40 has been screened.

1.3 Preparation of Immunogen C

The cDNA encoding the full-length amino acid sequence of human OX40 (sequence is from Uniprot database, protein sequence number is P43489) was cloned into pCP vector (pCP-hOX40 plasmid) and coated into 1.0 μm gold colloidal bullet (purchased from Bio-rad), That is, immunogen C was obtained. Immunization with Helios Gene Gun (Helios Gene Gun System, Bio-rad, article number 165-2431). Among them, the method of coating the 1.0 μm gold colloidal bullet and immunization can be found in the instruction manual of Helios gene gun.

1.4 Construction of a Stable Cell Line Expressing OX40 Protein

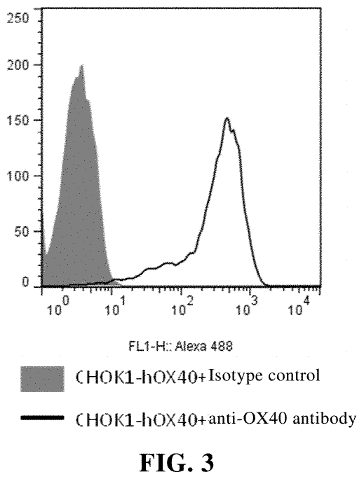

The pLVX-hOX40-IRES-Puro plasmid in Example 1.2 was transfected into the CHOK1 cell line, and subcloned in a 96-well culture plate by the limiting dilution method. Selectively cultivated in F12K medium containing 10% fetal bovine serum containing 6 μg/ml puromycin. After about 2 weeks, some monoclonal wells were selected and expanded into 24-well plates, and after about 3-4 days, they were expanded into 6-well plates. The amplified clones were screened by flow cytometry with OX40 antibody (purchased from R&D). The cell line with better growth, higher fluorescence intensity, and monoclonal cell line was selected to continue to expand and culture and cryopreserved in liquid nitrogen to obtain a stable CHOK1 cell line expressing human OX40 protein (herein referred to as CHOK1-hOX40 stable cell line). FIG. 3 shows that a stable CHOK1 cell line stably expressing human OX40 has been screened.

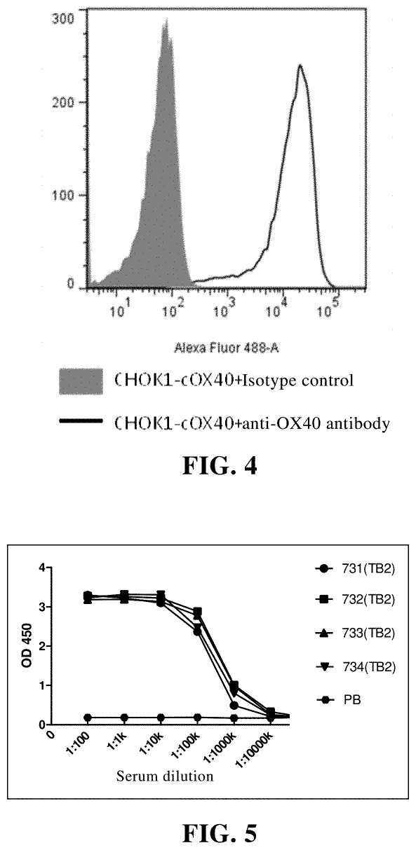

The nucleotide sequence encoding the full-length amino acid sequence of monkey-derived OX40 (the monkey-derived OX40 sequence is from NCBI database, the sequence number is XP_005545179.1) was cloned into the pLVX-IRES-Puro vector and the plasmid (herein referred to as pLVX-cOX40-IRES-Puro) was prepared. The pLVX-cOX40-IRES-Puro plasmid was transfected into the CHOK1 cell line, and the CHOK1 stable cell line expressing monkey OX40 (herein referred to as the CHOK1-cOX40 stable cell line) was screened. FIG. 4 shows that a stable CHOK1 cell line stably expressing monkey OX40 has been screened.

1.5 Preparation of Hybridoma Cells and Screening of Antibodies

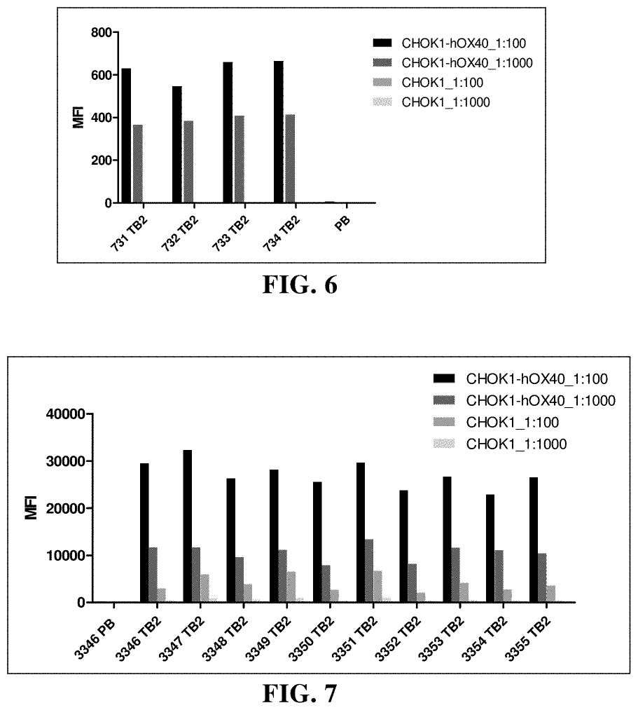

A. Immunogen A was used to immunize Balb/c and SJL mice (purchased from Shanghai SLAC) at the age of 6-8 weeks, and the mice were raised under SPF conditions. During the initial immunization, immunogen A protein was emulsified with Freund's complete adjuvant and injected intraperitoneally with 50 μg. When subjected to booster immunization, immunogen A protein was emulsified with Freund's incomplete adjuvant and injected intraperitoneally with 25 μg. There is an interval of 2 weeks between the initial immunization and the first booster immunization, and an interval of 3 weeks between each subsequent booster immunization. Blood was collected one week after each booster immunization, and the antibody titer and specificity of immunogen A in the serum were detected by ELISA and FACS. The results are shown in FIG. 5 and FIG. 6. FIGS. 5 and 6 show that the serum of mice immunized with OX40 ECD-hFc have different levels of binding to the immunogen. The antibody titer of serum detected by ELISA is more than 1:100,000, and the titer of serum antibody detected by FACS is more than 1:1000. Among them, TB2 refers to the mice serum seven days after the second booster immunization, and PB refers to the mice serum before the first immunization. The data in the figure are the OD450 nm value and the mean fluorescence intensity (MFI value), respectively.

B. Immunogen B was used to immunize Balb/c and SJL mice aged 6-8 weeks (purchased from Shanghai SLAC). After receiving the mice, they were raised under SPF conditions. The HEK293 cells (HEK293-hOX40) stably expressing human OX40 protein screened in the step (2) of Example 1 were expanded and cultured to 90% confluence in a cell culture dish, the medium was removed, washed with PBS, and adding cell digestion solution TrypLE (purchased from Invitrogen) and treated at 37° C. until the cells can be detached from the wall of the culture dish, and then collecting the cells. Washed twice with PBS, counting the cells and resuspended with PBS to 1-2×10⁷ cells per milliliter. Each mouse was intraperitoneally injected with 0.5 ml of cell suspension during each immunization. There is an interval of 2 weeks between the first and second immunizations, and an interval of 3 weeks between each subsequent immunization. Except for the first immunization, blood was collected one week after each immunization, and the antibody titer and specificity in the serum were detected by flow cytometry (FACS). The result is shown in FIG. 7. FIG. 7 shows that after the second booster immunization, the serum antibody titer detected by FACS can reach more than 1:1000. Among them, TB2 refers to the mice serum seven days after the second booster immunization, and PB refers to the mice serum before the first immunization. The data in the figure is the mean fluorescence intensity (MFI).

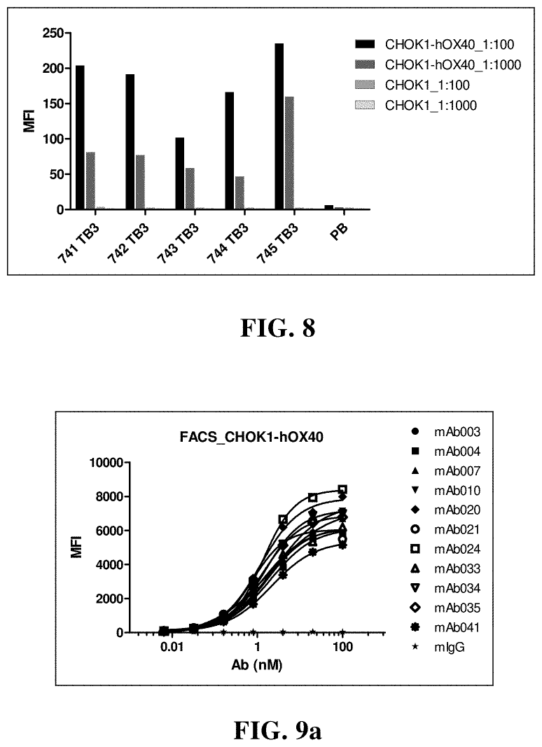

C. Immunogen C was used to immunize 6-8 weeks old Balb/c and SJL mice (purchased from Shanghai SLAC). After receiving the mice, they were raised under SPF conditions. All mice were immunized with Helios gene gun through the abdomen for 4 times, 4 shots each time, 1.0 μg cDNA per shot. The interval between the initial immunization and the first booster immunization is 2 weeks, and the interval between each subsequent booster immunization is 2 weeks. Blood was collected 1 week after each booster immunization, and the antibody titer in the serum was detected by FACS. The result is shown in FIG. 8. FIG. 8 shows that after the third booster immunization, the serum antibody titer detected by FACS can reach more than 1:1000. Among them, TB3 refers to the mice serum seven days after the third booster immunization, and PB refers to the mice serum before the first immunization. The data in the figure is the mean fluorescence intensity (MFI).

After steps A to C were completed, selecting mice with better serum antibody titer for fusion. For mice that were immune to immunogen A and immunogen C, 25 μg of immunogen A (OX40 ECD-hFc recombinant protein) was injected into the enterocoelia for booster immunization before fusion; Mice that have been immune to immunogen B were injected with immunogen B (0.5-1×10⁷ HEK293-hOX40 cells) into the enterocoelia before fusion for booster immunization. After 5 days, the mice were sacrificed, spleen cells were collected, and a spleen cell suspension was obtained. The cells were washed by centrifugation with DMEM basal medium for 3 times, and then mixed with mouse myeloma cells SP2/0 at a ratio of 5:1 in the number of viable cells, and the PEG method (see METHODS IN ENZYMOLOGY, VOL. 220) was used for cell fusion. The fused cells were diluted into DMEM medium containing 20% fetal bovine serum and 1×HAT (purchased from Invitrogen). Then adding 1×105/200 μL per well to a 96-well cell culture plate, and putting it in a 5% CO2, 37° C. incubator. After 10-14 days, using ELISA or Acumen (microplate cell detection method) to determine the binding activity of the cell fusion plate supernatant to OX40 protein, and selecting positive clones with higher OD450 nm value in ELISA or higher mean fluorescence intensity (MFI) in Acumen to expand into 24-well plate, and expanded culture in DMEM medium containing 10% fetal bovine serum and 1×HT (purchased from Invitrogen). After culturing for 3 days, taking the supernatant from the 24-well plate, using ELISA and FACS for secondary screening, determining the binding activity of the antibody in the supernatant to the OX40 protein, and the reporter gene experiment was used to detect the activation of NF-kB signaling pathway by antibodies in the supernatant. (see Example 3.2.2 for the method).

According to the results of the 24-well plate screening, the positive clones that have antigen-antibody binding activity and can activate the NF-kB signal pathway in ELISA and FACS experiments were selected, and subcloned in a 96-well plate by the limiting dilution method. Cultured in DMEM medium containing 10% FBS at 37° C. and 5% CO2. Seven days after subcloning, preliminary screening was carried out by ELISA or Acumen, and 4 positive monoclonals were selected and amplified to 24-well plates for further culture. After 3 days, the binding activity was determined by ELISA and FACS.

According to the test results of the 24-well plate samples, the optimal clone was selected, and the hybridoma cells of the present invention were obtained by expanding the culture in DMEM medium containing 10% FBS at 37° C. and 5% CO2, and cryopreserving in liquid nitrogen, and can be used for subsequent antibody production and purification.

### Example 2 Production and Purification of Lead Antibody

The antibody concentration produced by hybridoma cells is low, only about 0.1-10 μg/mL, and the concentration varies greatly. In addition, the various proteins produced by cell culture in the medium and the fetal bovine serum components contained in the medium interfere with many biological activity analysis methods to varying degrees, so that small-scale (1-5 mg) antibody production and purification is required.

The hybridoma cells obtained in Example 1 were inoculated into T-75 cell culture flasks, and using DMEM medium containing 10% FBS for expansion culture. When it grows well, inoculating it to the cell culture spinner flask and using a production medium containing 2.5% FBS (Low IgG) (Hybridoma serum free medium, purchased from Invitrogen). Adding 500 mL production medium to each 2 L culture spinner flask, and the inoculation cell density is 1.0×10⁵/mL. Tightly cap the bottle, and placing the spinner flask on the rotary machine in the 37° C. incubator at a speed of 3 revolutions per minute. After 14 days of continuous rotation culture, the cell culture solution was collected and filtered with a 0.22 μm filter membrane until the culture supernatant was clarified. The clarified culture supernatant can be purified immediately or frozen at −30° C.

The monoclonal antibody in the culture supernatant of the clarified hybridoma cells was purified with a protein A column (purchased from GE Healthcare). The protein A column was first equilibrated with PBS phosphate buffer, and then the clarified culture supernatant was loaded onto the protein A column. After loading the sample, washing the protein A column with equilibration buffer (0.1 M Tris-HCl, pH 8.0). Elution solution (0.1 M sodium citrate buffer, pH 4.5 for IgG1, pH 3.5 for other IgG subtypes) was used to elute the OX40 antibody bound to the protein A column. Collecting the eluted antibody, adding 1.0M Tris-HCl buffer to neutralize the pH, and then dialyzed against PBS phosphate buffer overnight. The OX40 antibody after dialysis was collected, sterile filtered with a 0.22 μm filter, subjected to sterile preservation to obtain purified OX40 antibody.

The purified OX40 antibody was tested and analyzed for protein concentration, purity, and endotoxin (Lonza kit). The results are shown in Table 4.

TABLE 4
Detection and analysis of purified OX40 antibody

| | | Concen- | | | |
|---|---|---|---|---|---|
| Antibody | | tration | SDS- | | Endotoxins |
| name | Subtype | (mg/ml) | PAGE | SEC | (EU/mg) |
| MAb003 | IgG1, k | 1.584 | >95% | 100% | 0.71 |
| MAb004 | IgG1, k | 0.866 | >95% | 100% | 1.32 |
| MAb007 | IgG2b, k | 0.637 | >95% | 100% | 2.00 |
| MAb010 | IgG1, k | 1.707 | >95% | 100% | 0.61 |
| MAb020 | IgG2c, k | 2.542 | >95% | 100% | 0.36 |
| MAb021 | IgG2b, k | 2.597 | >95% | 100% | 0.40 |
| MAb024 | IgG2c, k | 1.345 | >95% | 100% | 0.84 |
| MAb026 | IgG1, k | 1.906 | >95% | 97.97% | 0.37 |
| MAb033 | IgG1, k | 2.011 | >95% | 97.66% | 0.36 |
| MAb034 | IgG1, k | 1.07 | >95% | 100% | 1.10 |
| MAb035 | IgG1, k | 2.032 | >95% | 97.09% | 0.42 |
| MAb041 | IgG1, k | 1.635 | >95% | 100% | 0.53 |
| MAb047 | IgG2c, k | 0.842 | >95% | 100% | 1.89 |
| MAb049 | IgG1, k | 0.248 | >95% | 100% | 6.43 |
| MAb053 | IgG2c, k | 1.374 | >95% | 100% | 0.96 |
| MAb054 | IgG1, k | 1.243 | >95% | 100% | 1.13 |
| MAb055 | IgG1, k | 0.292 | >95% | 100% | 6.08 |
| MAb058 | IgG1, k | 1.616 | >95% | 100% | 0.08 |
| MAb064 | IgG2c, k | 1.768 | >95% | 100% | 0.07 |
| MAb069 | IgG1, k | 1.043 | >95% | 100% | 0.12 |

### Example 3 Detection of Lead Antibody

3.1 Flow Cytometry (FACS) to Detect the Binding of Antibodies to Cells Stably Expressing OX40 Protein

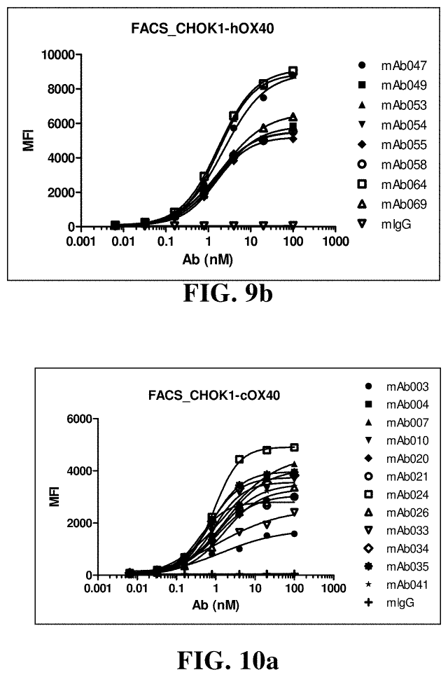

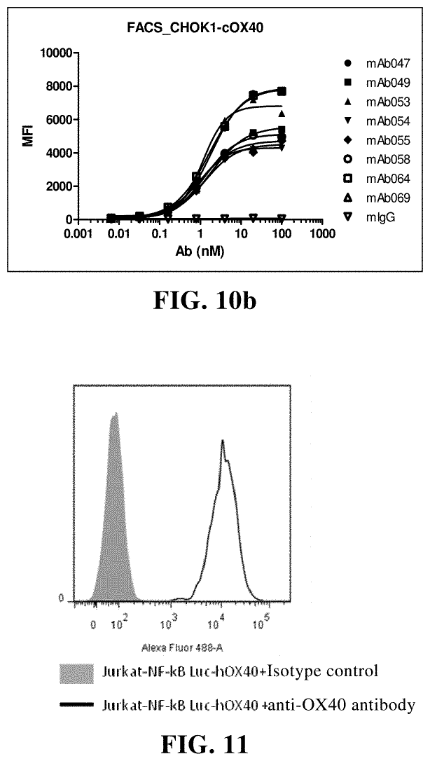

The CHOK1-hOX40 stable cell line and the CHOK1-cOX40 stable cell line constructed in Example 1.4 were expanded to 90% confluence in a cell culture dish, the medium was removed, washed with PBS phosphate buffer, and then the cells were treated and collected with cell digestion solution TrypLE (purchased from Invitrogen). Washing the cells with FACS buffer (PBS+2% FBS). After counting the cells, resuspending the cells in FACS buffer to 3×10⁶ cells/ml, adding 100 μL per well to a 96-well FACS reaction plate, and centrifuged at 300 g for 5 minutes and removing the supernatant, and a series of concentration gradients of 100 μL of the purified OX40 antibody obtained in Example 2 were added to each well, and incubated at 4° C. for 1 hour. Washed twice with FACS buffer by centrifugation, adding 100 μL fluorescent (Alexa 488) labeled secondary antibody (purchased from Invitrogen) per well, and incubated for 1 hour at 4° C. in the dark. Washing the cells by centrifugation with FACS buffer for 3 times, adding 100 μL of PBS buffer per well to suspend the cells, using FACS (FACS Canto II, purchased from BD) to detect and analyze the results. The results are shown in FIG. 9a, FIG. 9b and FIG. 10a, FIG. 10b. The antibody to be tested can bind to the OX40 protein on the cell surface. Among them, mIgG is the antibody subtype control mouse IgG, and the data MFI in the figure is the mean fluorescence intensity value of the measured cell population.

3.2 the NF-kB Reporter Gene Experiment Detects the Activation of OX40 Antibody on the NF-kB Signaling Pathway

3.2.1 Construction of Jurkat-NF-kB Luc-hOX40 Stable Cell Line

The pGL4.32[luc2P/NF-κB-RE/Hygro] plasmid (purchased from Promega) was transfected into Jurkat cells, cultured in RPMI1640 medium containing hygromycin and 10% fetal bovine serum, and Jurkat cell line stably expressing NF-kB RE-luciferase (herein referred to as Jurkat-NF-kB Luc) was screened and obtained.

The pLVX-hOX40-IRES plasmid in Example 1.2 was transfected into Jurkat-NF-kB Luc cells and cultured with RPMI1640 medium containing both hygromycin, puromycin and 10% fetal bovine serum, the Jurkat cell line stably expressing NF-kB RE-luciferase and OX40 was screened and obtained (herein referred to as Jurkat-NF-kB Luc-hOX40).

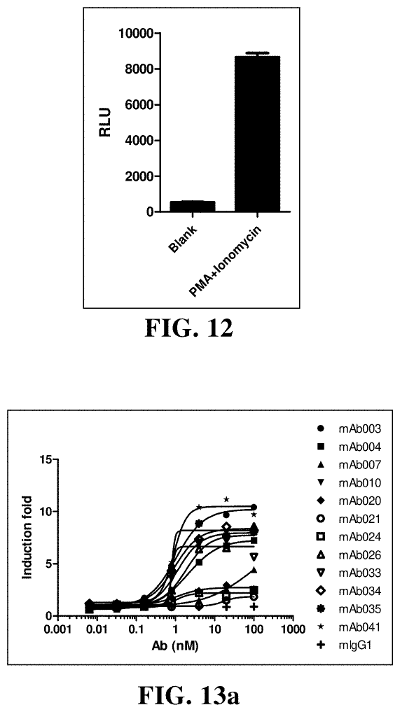

FIG. 11 shows the results of FACS detection, indicating that the screened and obtained Jurkat-NF-kB Luc-hOX40 cell line stably expresses hOX40. FIG. 12 shows the results of the NF-kB reporter gene experiment, indicating that the screened and obtained Jurkat-NF-kB Luc-hOX40 cell line stably expresses NF-kB RE-luciferase.

3.2.2 NF-kB Reporter Gene Experiment

Pre-coated a 96-well plate (purchased from Perkin Elmer) with 1 ug/ml anti-CD3 antibody, and incubated overnight at 4° C. Washed three times with PBS before use.

The Jurkat-NF-kB Luc-hOX40 cells screened in 3.2.1 were expanded and cultured. After collection, they were resuspended in RPMI1640 medium containing 1% FBS. Inoculating the cells in a 96-well plate coated with anti-CD3 antibody at a density of 1×10⁵/well. At the same time, 1×10⁵/well Raji cells were added as Fc gamma receptor expression cells. Then adding a series of concentration gradient OX40 antibody, putting it into the cell culture incubator and continuing to culture for 5h. The content of luciferase was determined with One-Glo Luciferase Detection System (purchased from Promega).

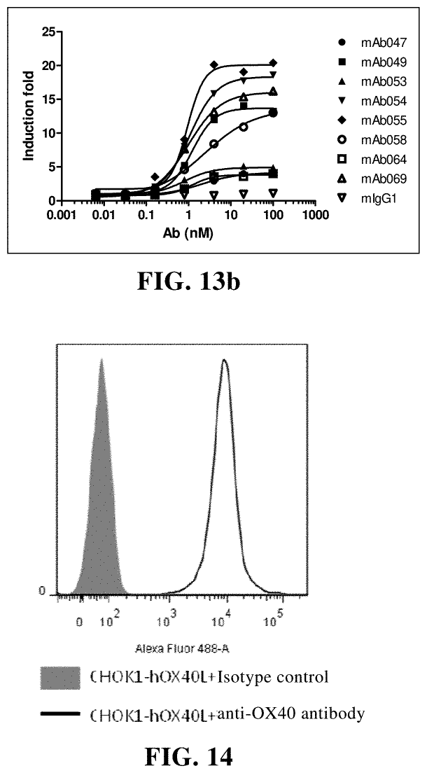

The results are shown in FIG. 13a and FIG. 13b, and the results show that the antibody can significantly activate the NF-kB signaling pathway.

3.3 T Cell Activation Test to Detect the Activation of T Cells by OX40 Antibody

3.3.1 Isolation of Peripheral Blood Mononuclear Lymphocytes PBMC from Human Whole Blood

The freshly obtained human whole blood was diluted with phosphate buffer PBS at a volume ratio of 1:1 to obtain the diluted whole blood, and the diluted whole blood was gently spread on the surface of Ficoll (purchased from GE Healthcare) with a sterile pipette, the volume ratio of Ficoll to diluted whole blood is 3:4. Avoiding shaking and mixing. Centrifuged at 400 g at room temperature and 20° C. gradient for 30 minutes. The centrifuge tube after centrifugation is divided into four layers, the second layer of milky white from top to bottom is mononuclear lymphocytes. Using a sterile pipette to gently absorb the cells in this layer, collecting them in a new centrifuge tube, diluting to three times the volume with PBS phosphate buffer, centrifuged at 200 g for 10 minutes at room temperature, and discarding the supernatant. The lymphocytes were resuspended to 10 mL in PBS phosphate buffer, and the previous steps were repeated and washed twice to obtain peripheral blood mononuclear lymphocytes PBMC.

3.3.2 Isolation of Human Memory CD4+ T Cells

Purified memory CD4+ T cells were isolated from the human peripheral blood mononuclear lymphocyte PBMC obtained in step 3.3.1 with the human memory CD4+ T cell extraction kit (purchased from Stem Cell, article number 19157). Refer to the kit instructions for the extraction method.

3.3.3 T Cell Activation Experiment

Pre-coated a 96-well plate with 1 ug/ml anti-CD3 antibody and 2.2 ug/ml AffiniPure F(ab′)2 Fragment Goat Anti-Mouse IgG (purchased from Jaskson Immunoresearch), and incubated overnight at 4° C. Washed three times with PBS before use.

Diluting the sample solution to be tested with multi-component RPMI1640 medium (purchased from Invitrogen) containing 10% FBS and adding 100 uL each well to the pre-coated 96-well plate. Then spreading the memory CD4+ T cells obtained in step 3.3.2 to a pre-coated 96-well plate with 1×10⁵ cells 100 uL per well. After the reaction plate was incubated in a 37° C., 5% CO2 incubator for 72 hours, the supernatant was collected and stored at −20° C. for the determination of IFN gamma content.

3.3.4 Detection of Cytokine IFN Gamma Content in Cell Supernatant

The detection of cytokine IFN gamma content in the cell supernatant uses the R&D system detection kit Human IFN-gamma DuoSet ELISA (DY285), and operates in accordance with the instructions.

Detecting the effect of OX40 antibody on the secretion of IFN gamma in the T cell activation experiment described in Example 3.3.2. The results are shown in Tables 5-9. In the T cell activation experiment, the antibody to be tested can enhance the secretion of IFN gamma from memory CD4+ T cells. Among them, mIgG1 is the antibody subtype control, and the data in the table is the IFN gamma value (pg/mL).

TABLE 5
The effect of OX40 antibody on IFN gamma secretion
in T cell activation experiments (PBMC donor-1)

| | IFN gamma (pg/ml) | | | | |
|---|---|---|---|---|---|
| | Antibody concentration(ug/ml) | | | | |
| Antibody name | 0.0016 | 0.008 | 0.04 | 0.2 | 1 |
| MAb007 | 2036.4 | 1480.8 | 1673.4 | 2161.2 | 2039.6 |
| MAb020 | 1809.3 | 2755.2 | 2437.3 | 2636.0 | 2373.2 |
| MAb021 | 1596.5 | 1427.1 | 1694.2 | 2613.3 | 2354.3 |
| MAb024 | 1343.8 | 1706.3 | 1945.9 | 1709.6 | 2103.7 |
| MIgG1 | 341.2 | 475.5 | 610.5 | 632.6 | 928.0 |

TABLE 6
The effect of OX40 antibody on IFN gamma secretion
in T cell activation experiments (PBMC donor-2)

| | IFN gamma (pg/ml) | | | | |
|---|---|---|---|---|---|
| | Antibody concentration(ug/ml) | | | | |
| Antibody name | 0.0016 | 0.008 | 0.04 | 0.2 | 1 |
| MAb026 | 1167.1 | 1662.4 | 2258.7 | 3751.4 | 5438.1 |
| MAb033 | 1461.7 | 3008.5 | 5224.1 | 4399.3 | 4150.6 |
| MAb034 | 1299.4 | 2898.1 | 3082.7 | 2686.0 | 3068.5 |
| MAb035 | 1426.5 | 2609.4 | 3569.3 | 4245.3 | 4233.9 |
| MIgG1 | 794.2 | 818.2 | 1079.1 | 967.3 | 896.6 |

TABLE 7
The effect of OX40 antibody on IFN gamma secretion
in T cell activation experiment (PBMC donor-3)

| | IFN gamma (pg/ml) | | | | |
|---|---|---|---|---|---|
| | Antibody concentration(ug/ml) | | | | |
| Antibody name | 0.0016 | 0.008 | 0.04 | 0.2 | 1 |
| MAb041 | 1895.0 | 2909.2 | 5449.0 | 7004.6 | 4930.4 |
| MAb047 | 1927.1 | 2197.8 | 4849.9 | 4775.0 | 5078.2 |
| MAb053 | 1063.7 | 1888.2 | 3226.6 | 3974.0 | 2598.6 |
| MAb064 | 905.8 | 1848.6 | 2631.4 | 2981.4 | 3259.2 |
| MIgG1 | 642.5 | 778.7 | 686.9 | 596.8 | 1515.9 |

TABLE 8
The effect of OX40 antibody on IFN gamma secretion
in T cell activation experiment (PBMC donor-4)

| | IFN gamma (pg/ml) | | | | |
|---|---|---|---|---|---|
| | Antibody concentration(ug/ml) | | | | |
| Antibody name | 0.0016 | 0.008 | 0.04 | 0.2 | 1 |
| MAb049 | 2150.0 | 2940.8 | 5250.2 | 5721.2 | 5438.3 |
| MAb054 | 2044.2 | 4887.3 | 5316.9 | 5220.4 | 6739.3 |
| MAb055 | 1623.4 | 3051.0 | 4368.1 | 3738.8 | 5062.9 |
| MAb058 | 2159.8 | 4575.3 | 5937.3 | 4121.1 | 3870.2 |
| MAb069 | 2116.8 | 3128.5 | 4109.4 | 4480.5 | 4800.2 |
| MIgG1 | 602.7 | 811.1 | 451.8 | 408.4 | 965.5 |

TABLE 9
The effect of OX40 antibody on IFN gamma secretion
in T cell activation experiment (PBMC donor-5)

| | IFN gamma (pg/ml) | | | | |
|---|---|---|---|---|---|
| | Antibody concentration(ug/ml) | | | | |
| Antibody name | 0.0016 | 0.008 | 0.04 | 0.2 | 1 |
| MAb003 | 3377.0 | 5217.5 | 6024.6 | 5636.0 | 6609.7 |
| MAb004 | 2709.0 | 3803.9 | 5892.4 | 4810.3 | 6788.2 |
| MAb010 | 2844.8 | 3993.7 | 4615.4 | 3955.6 | 3448.4 |
| MIgG1 | 1413.2 | 1710.2 | 1716.4 | 1116.1 | 1208.1 |

3.4 the Inhibitory Effect of OX40 Antibody on the Binding of OX40 and OX40L Proteins

3.4.1 Construction of a Cell Line Stably Expressing OX40L

The nucleotide sequence encoding the full-length amino acid sequence of human-derived OX40L (the protein sequence number of human-derived OX40L in the Uniprot database is P23510) was cloned into the pLVX-IRES-Puro vector and the plasmid (herein referred to as pLVX-hOX40L-IRES-Puro) was prepared. After plasmid transfection of the CHOK1 cell line, it was subcloned in a 96-well culture plate by the limiting dilution method and selectively cultured in F12K medium containing 10% fetal bovine serum containing 10 μg/ml puromycin, after about 2 weeks, selecting part of the monoclonal wells to be expanded into a 24-well plate, and about 3-4 days later to be expanded into a 6-well plate. The amplified clones were screened by flow cytometry with OX40L antibody (purchased from R&D). Choosing a cell line with better growth, higher fluorescence intensity, and monoclonal cell lines to continue to expand the culture and cryopreserve in liquid nitrogen to obtain a cell line stably expressing OX40L (herein referred to as CHOK1-hOX40L cells).

The FACS test results are shown in FIG. 14. The results show that the selected cell line can highly express hOX40L.

3.4.2 the Inhibitory Effect of OX40 Antibody on the Binding of OX40 and OX40L Protein

The CHOK1-hOX40L stable cell line constructed in Example 3.4.1 was expanded and cultured in a cell culture dish to 90% confluence, the medium was removed, washed with PBS, the cells were treated and collected with cell digestion solution TrypLE (purchased from Invitrogen). Washing the cells with FACS buffer (PBS+2% FBS). After counting the cells, resuspending the cells to 3×10⁶/mL in FACS buffer, adding 100 μL per well to a 96-well FACS reaction plate, and centrifuged at 300 g for 5 minutes and the supernatant was removed, a series of concentration gradients of 50 μL of the purified OX40 antibody obtained in Example 2 were added to each well, and at the same time, adding 50 μL of biotin-labeled OX40 ECD-Fc protein per well, and incubated at 4° C. for 1 hour. Washed twice with FACS buffer by centrifugation, adding 100 μL fluorescent (Alexa 488)-labeled streptavidin (purchased from Invitrogen) per well, and incubated for 1 hour at 4° C. in the dark. Washing the cells by centrifugation with FACS buffer for 3 times, adding 100 μL PBS buffer per well to suspend the cells, using FACS (FACS Canto II, purchased from BD) to detect and analyze the results.

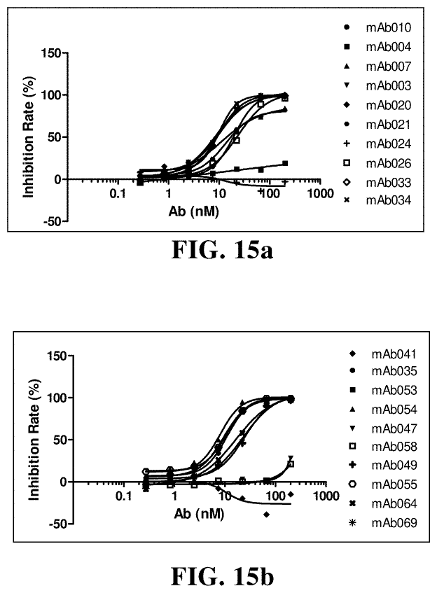

The results are shown in FIG. 15a and FIG. 15b. The results show that most of the antibodies of the present invention can inhibit the binding of OX40 and OX40L proteins.

### Example 4 Determination of the Amino Acid Sequence of the Variable Region of the Light and Heavy Chains

Isolation of total RNA: After the supernatant obtained from the subclonal culture in Example 1 was tested for antigen binding (that is, after the verification and activity determination in Example 3), 5×10⁷ hybridoma cells were collected by centrifugation, and 1 mL Trizol was added and mixed well and transferred to a 1.5 mL centrifuge tube, placed at room temperature for 5 minutes; adding 0.2 mL chloroform, shaken for 15 seconds, placed for 2 minutes, centrifuged at 12000 g at 4° C. for 5 minutes, taking the supernatant and transferred to a new 1.5 mL centrifuge tube; adding 0.5 mL isopropanol, gently mixing the liquid in the tube, placed at room temperature for 10 minutes, centrifuged at 4° C., 12000 g for 15 minutes, discarding the supernatant; adding 1 mL of 75% ethanol, gently washing the precipitate, 4° C., after centrifugation at 12000 g for 5 minutes, the supernatant was discarded, the precipitate was air-dried, and DEPC-treated H₂O was added to dissolve it (55° C. water bath to promote solvent for 10 minutes) to obtain total RNA.

Reverse transcription and PCR: Taking 1 μg of total RNA, configuring a 20 μL system, adding reverse transcriptase and reacted at 42° C. for 60 minutes, and at 7° C. for 10 minutes to terminate the reaction. Configuring 50 μL PCR system, including 1 μL cDNA, 25 pmol of each primer, 1 μL DNA polymerase and matching buffer system, 250 μmol dNTPs; setting up PCR program, 95° C. pre-denaturation for 3 minutes, 95° C. denaturation for 30 seconds, annealed at 55° C. for 30 seconds, extended at 72° C. for 35 seconds, and then extended at 72° C. for an additional 5 minutes after 35 cycles to obtain a PCR product. The kit used for reverse transcription is PrimeScript RT Master Mix, purchased from Takara, item number RR036; the kit used for PCR includes Q5 ultra-fidelity enzyme, purchased from NEB, item number M0492.

Cloning and sequencing: Taking 5 μl of PCR product for agarose gel electrophoresis detection, and using the column recovery kit to purify the test positive samples. The recovery kit is Gel&PCR Clean-up, purchased from MACHEREY-NAGEL, catalog number 740609. Carrying out ligation reaction: sample 50 ng, T vector 50 ng, ligase 0.5 μL, buffer 1 μL, reaction system 10 μL, reacted at 16° C. for half an hour to obtain the ligation product, the ligation kit is T4 DNA ligase, purchased from NEB, catalog number M0402; Taking 5 μl of the ligation product and adding to 100 μL of competent cells (Ecos 101 competent cells, purchased from Yeastern, Catalog No. FYE607), ice bath for 5 minutes, then heat shock in a water bath at 42° C. for 1 minute, putting it back on ice for 1 minute and adding 650 μL antibiotic-free SOC medium, resuscitated on a 37° C. shaker at 200 rpm for 30 minutes, taking out 200 μL and spreading it on LB solid medium containing antibiotics and incubated overnight at 37° C. incubator; the next day, using primers M13F and M13R on the T vector to configure a 30 μL PCR system for colony PCR. Using a pipette tip to dip the colony into the PCR reaction system and pipette, and aspirate 0.5 μL and place it on another LB solid petri dish containing 100 nM ampicillin to save the strain; after the PCR reaction was over, taking out 5 μL for agarose gel electrophoresis detection, and sequencing and analysis of positive samples [see Kabat, “Sequences of Proteins of Immunological Interest”, National Institutes of Health, Bethesda, Md. (1991)].

See the sequence information of the present invention in the appendix for the sequencing results.

### Example 5 Epitope Identification of OX40 Antibody

According to the sequence in the published patent, we prepared four control antibodies, namely control antibody 1 (MOXR0916), control antibody 2 (PF-04518600), control antibody 3 (MEDI0562), and control antibody 4 (BMS-986178). The OX40 antibody obtained in Example 1 and the four control antibodies were respectively subjected to competitive ELISA experiments. The results are shown in Table 10. The percentage in the table is the percentage of competition. The higher the percentage of competition, the closer the epitopes of the two antibodies are. Among them, mAb007, mAb021, mAb024, mAb041, and mAb058 have different epitopes from the four control antibodies.

TABLE 10
Competitive ELISA experiment

| | Compete | Compete | Competing | Compete |
|---|---|---|---|---|
| Antibody | with | with | with | with |
| name | MOXR0916 | PF-04518600 | MEDI0562 | BMS-986178 |
| MAb010 | 56% | −19% | 96% | −6% |
| MAb049 | 90% | −2% | 97% | 0% |
| MAb064 | 90% | 4% | 96% | 1% |
| MAb069 | 73% | −1% | 96% | 1% |
| MAb004 | −2% | −12% | 92% | −8% |
| MAb026 | 3% | −13% | 63% | 11% |
| MAb035 | 3% | −5% | 59% | 90% |
| MAb047 | −3% | −2% | 97% | 0% |
| MAb003 | −2% | 94% | −3% | 5% |
| MAb053 | −2% | 93% | 11% | 2% |
| MAb054 | −2% | 95% | 11% | 18% |
| MAb055 | −3% | 95% | 16% | 3% |
| MAb020 | −4% | −15% | −3% | 50% |
| MAb033 | −1% | −7% | 7% | 81% |
| MAb034 | 0% | 5% | 13% | 93% |
| MAb007 | −2% | 16% | −3% | −6% |
| MAb021 | −2% | −10% | 3% | 7% |
| MAb024 | −1% | −7% | 9% | −2% |
| MAb041 | 1% | −8% | 2% | −6% |
| MAb058 | −3% | 11% | 26% | −4% |

### Example 6 Preparation and Identification of OX40 Human-Mouse Chimeric Antibody

6.1 Plasmid Construction and Preparation

Purified OX40 antibody has been obtained from the culture supernatant of hybridoma cells in Example 2, and according to the sequencing results of Example 4, the heavy chain variable region and light chain variable region sequences of the OX40 antibody have been identified. The heavy chain variable region sequence of the OX40 antibody was recombined into an expression vector containing the signal peptide and the human-derived heavy chain antibody IgG1 constant region, and the light chain variable region sequence of the OX40 antibody was recombined to an expression vector containing signal peptide and human-derived antibody light chain kappa constant region, a recombinant plasmid was obtained and verified by sequencing (the sequencing method is the same as that in Example 4). Using an alkaline lysis kit (purchased from MACHEREY-NAGEL) to extract the recombinant plasmids with improved purity with a mass of 500 μg or more, and filtering it through a 0.22 μm filter membrane (purchased from Millipore) for transfection.

6.2 Cell Transfection

Culturing 293E cells (purchased from Invitrogen) in Freestyle 293 expression medium (purchased from Invitrogen). The shaker is set to 37° C., 130RPM, and 8% CO2 (v/v) concentration.

In Freestyle 293 expression medium, 10% (v/v) F68 (purchased from Invitrogen) was added during transfection to a final concentration of F68 at 0.1% (v/v) to obtain Freestyle 293 expression culture containing 0.1% (v/v) F68, that is, medium A.

Taking 5 mL medium A and 200 μg/mL PEI (purchased from Sigma) and mixed well to obtain medium B. Taking 5 mL of medium A and 100 μg/mL of the recombinant plasmid obtained in step 6.1 and mixed well to obtain medium C. After 5 minutes, the medium B and the medium C were combined and mixed, and the mixture was allowed to stand for 15 minutes to obtain a mixture D. Adding 10 mL mixture D slowly to 100 mL Freestyle 293 expression medium containing 293E cells until the cell density of 293E was 1.5×10⁶/mL, and shaking while adding to avoid excessive concentration of PEI. Placing it in a shaker for culture. Peptone was added the next day to a final concentration of 0.5% (w/v). On the 5th to 7th day, the antibody titer of the culture medium was measured. On the 6th to 7th day, the supernatant was collected by centrifugation (3500 rpm, 30 minutes), and filtered through a 0.22 μm filter membrane to obtain the filtered cell supernatant for purification.

6.3 Antibody Purification and Identification

For continuous production of endotoxin-free chromatography columns and Protein A packing (purchased from GE), using 0.1M NaOH for 30 minutes or 5 times the column volume of 0.5M NaOH for washing; for column materials and chromatography columns that have not been used for a long time, using 1M NaOH to soak for at least 1 hour, rinsed with non-endotoxic water to neutrality, and washing the column material with 10 times the column volume of 1% Triton x 100. Using 5 column volumes of PBS (PBS phosphate buffer, pH 7.2) for equilibration, putting the filtered cell supernatant obtained in step 6.2 on the column, and collecting the flow-through if necessary. After loading the column, washed with 5 times the column volume of PBS. Eluted with 5 column volumes of 0.1M Glycine-HCl pH3.0, collecting the eluate, and neutralized with 0.5 times column volume eluent of 1M Tris-HCl (1.5M NaCl) with pH 8.5, and harvesting OX40 human mouse chimeric antibody. Dialysis in 1×PBS for 4 hours to avoid endotoxin contamination. After dialysis, using spectrophotometry or a kit to determine the concentration, using HPLC-SEC to determine the purity of the antibody, and using an endotoxin detection kit (purchased from Lonza) to detect the content of antibody endotoxin. The characteristics of the obtained OX40 human-mouse chimeric antibody were identified (same as Example 3).

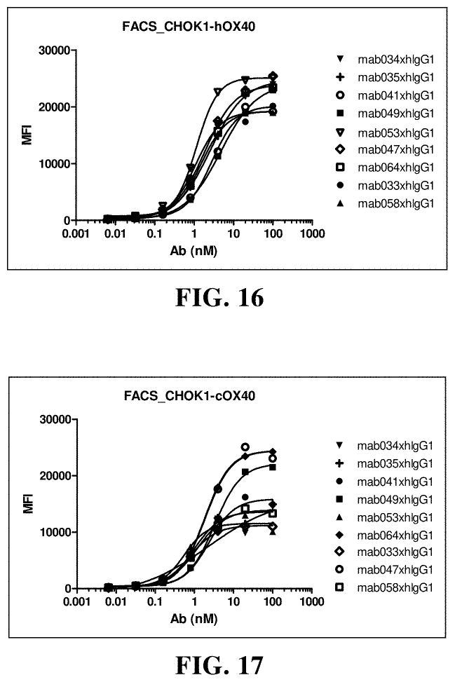

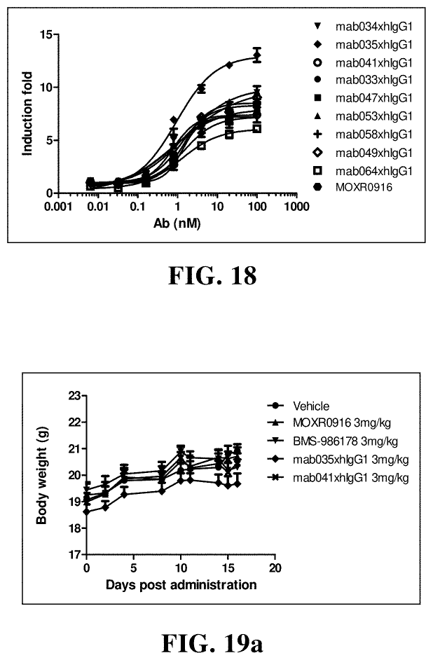

The test results are shown in FIG. 16, FIG. 17, FIG. 18 and Table 11. Wherein MOXR0916 is the positive control antibody, and hIgG1 is the antibody subtype control.

TABLE 11
Effect of OX40 human-mouse chimeric antibody on IFN
gamma secretion in T cell activation experiment

| | IFN gamma (pg/ml) | | | | | |
|---|---|---|---|---|---|---|
| | Antibody concentration (ug/ml) | | | | | |
| Antibody name | 1 | 0.2 | 0.04 | 0.008 | 0.0016 | 0 |
| MAb041 × hIgG1 | 4985.0 | 5071.4 | 6794.6 | 5352.7 | 2438.9 | 731.9 |
| MAb058 × hIgG1 | 3918.4 | 5054.8 | 5102.0 | 3992.2 | 3137.7 | 709.5 |
| MAb004 × hIgG1 | 4088.8 | 5074.6 | 4343.5 | 5612.0 | 3477.5 | 667.7 |
| MAb033 × hIgG1 | 4646.7 | 3502.5 | 5833.2 | 5032.4 | 2273.8 | 667.7 |
| MAb034 × hIgG1 | 4636.9 | 4673.1 | 4847.4 | 5191.5 | 3805.2 | 667.7 |
| MAb035 × hIgG1 | 5168.6 | 4993.0 | 4439.2 | 4737.1 | 4168.9 | 609.5 |
| MAb047 × hIgG1 | 5173.7 | 5704.0 | 6132.3 | 3252.5 | 1129.7 | 609.5 |
| MAb049 × hIgG1 | 3855.0 | 4722.6 | 4258.1 | 3943.8 | 1781.2 | 609.5 |
| MOXR0916 | 4469.0 | 3937.6 | 5007.3 | 2454.2 | 1418.5 | 709.5 |
| HIgG1 | 569.4 | 695.8 | 790.8 | 742.4 | 717.8 | 709.5 |

The results show that the obtained OX40 human-mouse chimeric antibody can bind well to both human OX40 and monkey OX40, can activate the NF-kB signaling pathway and can significantly increase the expression of IFN gamma in memory CD4+ T cells. Compared with the positive control antibody MOXR0916, mab035×hIgG1 has a better effect on activating T cells.

### Example 7 In Vivo Efficacy Experiment of OX40 Antibody

In order to study the anti-tumor effect of OX40 antibody in vivo, OX40 humanized mouse colon cancer MC-38 subcutaneous tumor model was established. OX40 humanized mice were purchased from Beijing Biocytogen Co., Ltd, female, 5-6 weeks old. 100 μL of cell suspension (cells suspended in base DMEM medium) containing 1×10⁶ MC-38 tumor cells was inoculated subcutaneously on the right side of the mouse. Animals with a tumor volume of 30 mm³-80 mm³ will be introduced into the experiment and randomly divided into different test groups according to the tumor volume. The animal experiment grouping and dosing schedule are shown in Table 12 below.

TABLE 12
Animal experiment grouping and dosing schedule

| | | Dose of | | | |
|---|---|---|---|---|---|
| | Name of | admin- | | Admin- | Admin- |
| | admin- | istration | Number | istration | istration |
| Group | istration | (mg/kg) | of mice | route | cycle |
| 1 | Control | / | 10 | Intra- | Twice a |
| | (Vehicle) | | | peritoneal | week for |
| 2 | MOXR0916 | 3 | 10 | injection | three weeks |
| 4 | BMS-986178 | 3 | 10 | | |
| 5 | Mab035 × | 3 | 10 | | |
| | hIgG1 | | | | |
| 6 | Mab041 × | 3 | 10 | | |
| | hIgG1 | | | | |

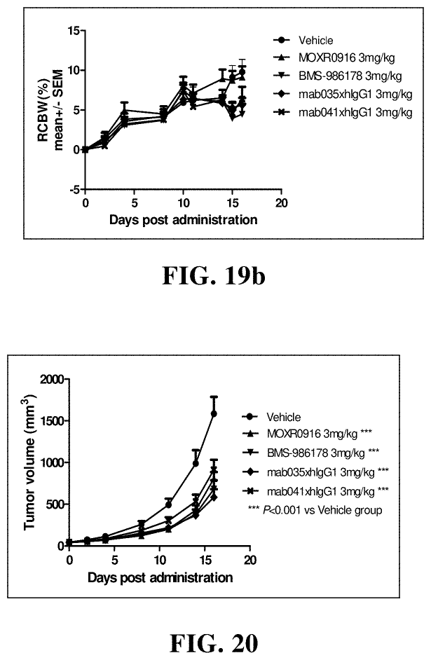

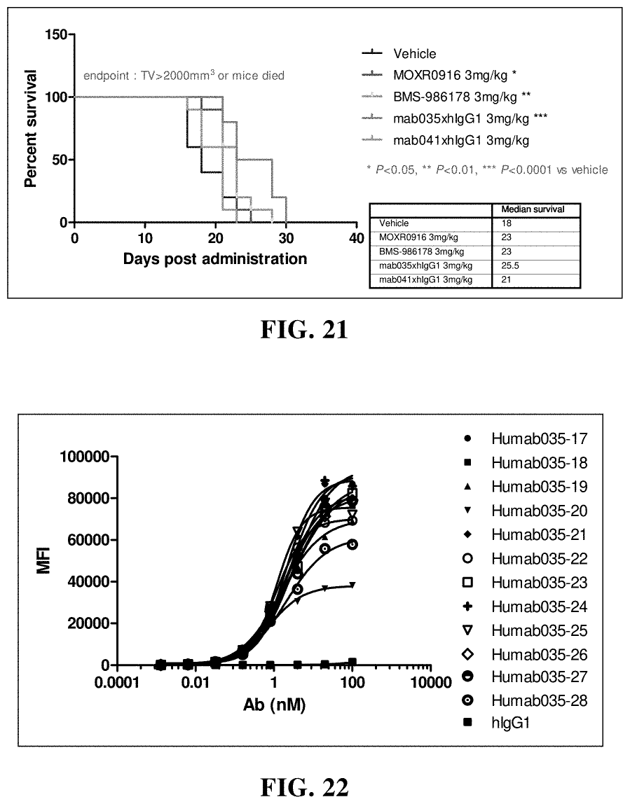

After the administration, the weight of the mice was recorded 2-3 times a week, and the tumor diameter was measured with a vernier caliper. The calculation formula of tumor volume is: V=a×b²/2, wherein a and b represent the long diameter and short diameter of the tumor, respectively. Each mouse reached the experimental endpoint (weight loss greater than 20%; or tumor volume greater than 2,000 mm³) and was euthanized and the survival time was recorded, Kaplan-Meier survival curve. Changes in body weight and tumor volume were analyzed by Two-way ANOVA, Kaplan-Meier survival curve was analyzed by Log-Rank, and P<0.05 was considered as a significant difference. The experimental results are shown in FIG. 19a, FIG. 19b, FIG. 20, and FIG. 21. Compared with the solvent control (vehicle) group, OX40 antibody can significantly inhibit tumor growth, and mab035×hIgG1 has the best effect on prolonging the life cycle of mice.

### Example 8 Preparation of Humanized Antibodies

After sequence analysis, the candidate antibody mAb035 has no important hotspots in the heavy chain variable region and the light chain variable region. Through sequence alignment (NCBI-Igblast), the germline gene sequence with the highest homology of the heavy chain variable region and the variable region of the light chain of the candidate antibody mAb035 is selected as the variable region transplantation framework: IGHV1-46*03 (61.2%) and IGKV1-12*01 (67.4%). After selecting the human antibody framework, through homology modeling, predicting the key amino acids that may determine the structure in the mouse anti-constant region, and the grafted framework region is designed for back mutation.

According to the above principles, designing 10 heavy chain variable region sequences (mab035VH.g0, mab035VH.g1, mab035VH.g2, mab035VH.g3, mab035VH.g4, mab035VH.g5, mab035VH.g6, mab035VH.g7, mab035VH.g8, mab035VH.g9) and 5 light chain variable region sequences (mab035VL.g0, mab035VL.g1, mab035VL.g2, mab035VL.g3, mab035VL.g4). Subsequently, cross-combination was performed for expression, and a total of the following 28 humanized antibodies were obtained, as shown in Table 13.

TABLE 13
humanize antibody expression combinations

| | Mab035VL.g0 | Mab035VL.g1 | Mab035VL.g2 | Mab035VL.g3 | Mab035VL.g4 |
|---|---|---|---|---|---|
| Mab035VH.g0 | Humab035-1 | Humab035-2 | Humab035-3 | Humab035-4 | |
| Mab035VH.g1 | Humab035-5 | Humab035-6 | Humab035-7 | Humab035-8 | |
| Mab035VH.g2 | Humab035-9 | Humab035-10 | Humab035-11 | Humab035-12 | |
| Mab035VH.g3 | Humab035-13 | Humab035-14 | Humab035-15 | Hu Mab035-16 | |
| Mab035VH.g4 | | Hu Mab035-17 | | | Hu Mab035-23 |
| Mab035VH.g5 | | Hu Mab035-18 | | | Hu Mab035-24 |
| Mab035VH.g6 | | Hu Mab035-19 | | | Hu Mab035-25 |
| Mab035VH.g7 | | Hu Mab035-20 | | | Hu Mab035-26 |
| Mab035VH.g8 | | Hu Mab035-21 | | | Hu Mab035-27 |
| Mab035VH.g9 | | Hu Mab035-22 | | | Hu Mab035-28 |

Vector construction: The amplification primers were synthesized by Genewiz, and then the variable regions of the light chain and the heavy chain were amplified separately by PCR method. Configuring 50 μL reaction system, including 50-100 ng heavy chain variable region, light chain variable region, 1 ul forward and reverse primers, 1 ul pfxD enzyme (purchased from invitrogen, 12344-012), 10*pfx buffer 5 ul (Same supplier as pfx enzyme) and adding water to make up to 50 μL. Setting up the PCR program, pre-denaturation at 95° C. for 5 minutes, denaturation at 95° C. for 30 seconds, annealing at 56° C. for 30 seconds, extension at 68° C. for 30 seconds, after 25 cycles, an additional extension at 68° C. for 10 minutes to obtain a PCR product. Taking 5 μL of the PCR product for agarose gel electrophoresis detection, and using the recovery kit to purify the test positive samples. The recovery kit is PureLink Quick Gel extraction kit, purchased from Qiagen, catalog number 28706.

Preparation of humanized antibody: Carrying out ligation reaction: inserting fragment 20-40 ng, digested expression vector 60-10Ong, recombinase Exnase (purchased from Vazyme, catalog number C112-01/02) 1 μL, buffer 2 μL, the reaction system is 10 μL and reacted at 37° C. for half an hour to obtain the ligation product, which is the constructed recombinant vector. The buffer is the buffer used for the purchase of the recombinase; the heavy chain variable region is directional cloned into the expression vector containing the signal peptide and the human antibody heavy chain IgG4 (S228P) constant region (wherein the expression vector is purchased from Invitrogen, the recombination step is a normal step), the light chain variable region is directional cloned into an expression vector containing a signal peptide and a human antibody light chain kappa constant region (wherein the expression vector is purchased from Invitrogen, and the recombination step is a conventional step). Adding 10 μL of the ligation product to 100 μL of competent cells (Ecos 101 competent cells, purchased from Yeastern, Catalog No. FYE607), heat shock in a 42° C. water bath for 60 seconds, putting it back on ice for 3 minutes, taking out 80 μL and spreading it on the LB solid medium containing ampicillin, incubated overnight at 37° C. in an incubator. The next day, using the primers pEF1A and pSV40 on the expression vector to configure a 30 μL PCR system for colony PCR. The colony PCR system is: 1 μL for each primer, 15 μL of PCR master mix (purchased from Novoprotein), making up to 30 μL. Using a pipette tip to dip the colony into the PCR reaction system and pipette, and aspirating 0.5 μL onto another LB solid petri dish containing 100 μg/mL ampicillin to preserve the strain. After the PCR reaction, 4.5 μL was taken out for agarose gel electrophoresis detection, and the positive samples were sequenced.

The recombinant antibody heavy and light chain expression vectors with the correct sequence were amplified, and then transiently transfected into FreeStyle™ 293-F cells (purchased from Invitrogen) to produce antibodies. During transfection, the density of 293-F cells should be 1-1.2×10⁶ cells/mL, and for 100 mL of cells, 100 μg of the constructed recombinant vector and 200 μg of the transfection reagent polyethyleneimine (PEI) were required. The recombinant vector and PEI were added to 5 mL of culture medium, and the mixture was allowed to stand at room temperature for 5 minutes. After filtration with 0.22 μm filter membrane, the mixture of recombinant vector and PEI was allowed to stand at room temperature for 15 minutes. Then the above mixture was slowly added to the cells, and cultured in a 37° C., 8% (v/v) CO2 incubator at 130 rpm. The culture supernatant and cell pellet are taken every day to detect the expression of antibodies. After 5 days, the cell culture solution was centrifuged at 3000 g for 30 minutes, the supernatant was collected, and filtered with a 0.22 μm filter. Purifying the monoclonal antibody in 200 mL of clear supernatant with 1 mL MabSelect™ SuRe™ column (purchased from GE Healthcare). MabSelect™ SuRe™ column was first equilibrated with equilibration buffer (PBS phosphate buffer, pH7.2). After loading the sample, washing the MabSelect™ SuRe™ column with equilibration buffer. The volume of the equilibration buffer is 5 times the volume of the protein A column bed. The monoclonal antibody bound to the MabSelect™ SuRe™ column was eluted with an eluent (0.1 M glycine hydrochloride buffer, pH 3.0). Collecting the eluted antibodies and adding 10% (v/v) 1.0 M Tris-HCl buffer to neutralize the pH. Then immediately dialyzed with PBS phosphate buffer overnight. Collecting the dialyzed monoclonal antibodies, filtering them aseptically with a 0.22 μm filter, and storing them aseptically to obtain purified humanized OX40 antibodies. The obtained antibody was tested and analyzed for protein concentration and purity.

The results are shown in Table 14 below. The results show that the yield and purity analysis of humanized antibody are normal.

TABLE 14
Humanized antibody expression and purification results

| | | | Concen- | | |
|---|---|---|---|---|---|
| Antibody | Volume | batch | tration | Yield | Purity |
| name | (mL) | number | (mg/ml) | (mg) | (% SEC) |
| Humab035-1 | 1.8 | 2018071901 | 1.18 | 2.124 | 99.018 |
| Humab035-2 | 1.8 | 2018071902 | 1.261 | 2.270 | 99.367 |
| Humab035-3 | 1.9 | 2018072503 | 0.504 | 0.958 | 100 |
| Humab035-4 | 1.8 | 2018071904 | 1.51 | 2.718 | 99.592 |
| Humab035-5 | 1.7 | 2018071905 | 1.288 | 2.190 | 99.557 |
| Humab035-6 | 1.7 | 2018071906 | 1.012 | 1.720 | 99.696 |
| Humab035-7 | 1.8 | 2018071907 | 1.169 | 2.104 | 99.609 |
| Humab035-8 | 1.8 | 2018071908 | 1.186 | 2.135 | 99.872 |
| Humab035-9 | 1.8 | 2018071909 | 1.078 | 1.940 | 99.400 |
| Humab035-10 | 1.8 | 2018071910 | 1.171 | 2.108 | 99.358 |
| Humab035-11 | 1.8 | 2018071911 | 0.989 | 1.780 | 99.421 |
| Humab035-12 | 1.8 | 2018071912 | 1.206 | 2.171 | 99.718 |
| Humab035-13 | 1.8 | 2018071913 | 1.176 | 2.117 | 97.173 |
| Humab035-14 | 1.8 | 2018071914 | 0.813 | 1.463 | 95.878 |
| Humab035-15 | 1.8 | 2018072515 | 0.902 | 1.6236 | 96.390 |
| Humab035-16 | 1.8 | 2018071916 | 1.241 | 2.234 | 98.147 |
| Humab035-17 | 1.7 | 2018100901 | 2.569 | 4.367 | 97.88 |
| Humab035-18 | 1.7 | 2018100902 | 2.425 | 4.123 | 97.87 |
| Humab035-19 | 1.7 | 2018100903 | 2.868 | 4.876 | 97.36 |
| Humab035-20 | 1.7 | 2018100904 | 2.074 | 3.526 | 97.84 |
| Humab035-21 | 1.7 | 2018100905 | 2.456 | 4.175 | 97.68 |
| Humab035-22 | 1.7 | 2018100906 | 2.407 | 4.092 | 98.10 |
| Humab035-23 | 1.7 | 2018100907 | 2.279 | 3.874 | 98.56 |
| Humab035-24 | 1.7 | 2018100908 | 1.731 | 2.943 | 98.90 |
| Humab035-25 | 1.7 | 2018100909 | 2.232 | 3.794 | 98.66 |
| Humab035-26 | 1.7 | 2018100910 | 2.151 | 3.657 | 98.61 |
| Humab035-27 | 1.7 | 2018100911 | 2.306 | 3.920 | 98.58 |
| Humab035-28 | 1.7 | 2018100912 | 1.952 | 3.318 | 100.00 |

Activity identification of humanized antibody (the method is the same as in Example 3)

A. Flow cytometry (FACS) detects the binding of antibodies to cells expressing human OX40. The results are shown in FIG. 22. The antibodies obtained can all bind to the human-derived OX40 on the cell surface. The isotype control is human IgG1, and the data in the figure is the mean fluorescence intensity (MFI) of the measured cell population.

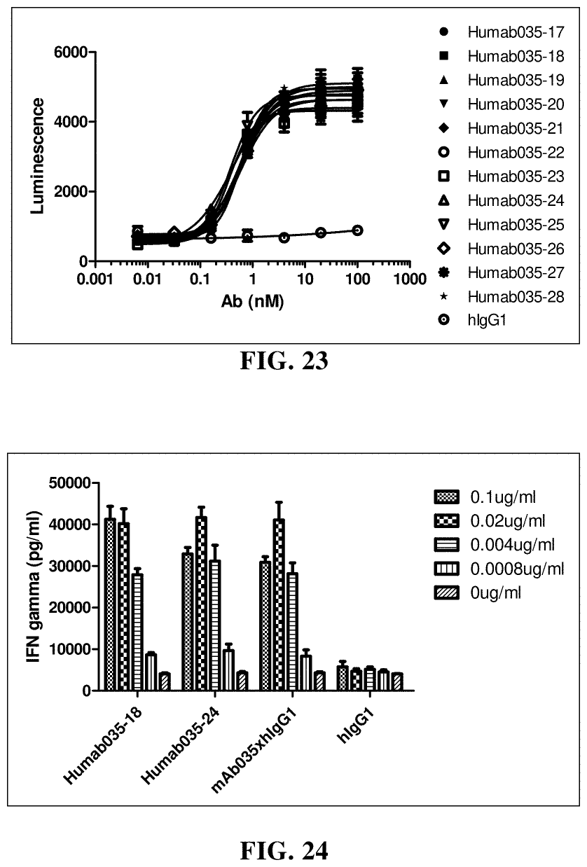

B. The NF-kB reporter gene experiment detects the activation of the antibody on the NF-kB signaling pathway. The results are shown in FIG. 23. The detection antibody can activate the NF-kB signaling pathway. The isotype control is human IgG1, and the data in the figure is luminous intensity (Luminescence).

C. The T cell activation test detects the activation effect of antibodies on T cells. The results are shown in FIG. 24. The detection antibody can significantly increase the expression of IFN gamma in memory CD4+ T cells and activate T cells. The isotype control is human IgG1, and the data in the figure is the content of IFN gamma in the supernatant.

All documents mentioned in the present invention are incorporated by reference herein as if each document were incorporated separately by reference. Furthermore, it should be understood that after reading the foregoing teachings of the invention, various changes or modifications may be made to the invention by those skilled in the art and that these equivalents are equally within the scope of the claims appended to this application.

### APPENDIX SEQUENCE INFORMATION OF THE PRESENT INVENTION

MAb035-H
SEQ ID NO. 1
Amino Acid Sequence (116aa)
QVQLQQSGAELVRPGASVTLSCKASDYTFTDYEMHWVYQTPVHGLEWIGA
IDPETGGTAYNQKFKDKAILTADKSSNTAYMELRSLTSEDSAVYFCTKTG
YQFDYWGQGTTLTVSS
>3626-mAb035-13-68A8G10-H (348 bp)
SEQ ID NO. 2
CAGGTTCAACTGCAGCAGTCTGGGCTGAGCTGGGGAGGCCTGGGCTTCAG
TGACGCTGCCTGCAAGGCTTCGGGCTTCCTGCAAGGCTTCGGACTGACTG
ACTATGAAATGCACTGGAAATGCACTGGCCTGGGAATGGAATGGCCTGGG
AATGGGAATTGCCTGGGAATGGCCTGGGAATTGGCCTGGGAATTGCCTGG
GAATTGCCTGGGAATTGCCTGGGGGGACATTGCCTGGGGGACATTGCCTG
GGCCGCAGCCTGGGACATGATTGCCTGGGGACATTGCCTGGGACATTGCC
TGGGGACATTGCCGCAGCCTGACTGACTGAGGACTCTGCCGTCTATTTCT
GTACAAAAACTGGGTACCAGTTTGGACTACTGGGGCCAAGGCACCACTCT
CTCCTCA

| | | | Sequence | |
|---|---|---|---|---|
| | | Position in | Number | |
| region | Sequence fragment | SEQ ID NO. 1 | SEQ ID NO. | Length |
| HFR1 | Amino acids 1-30 in SEQ ID NO. 1 | 1-30 | | 30 |
| CDR-H1 | DYEMH | 31-35 | 3 | 5 |
| HFR2 | Amino acids 36-39 in SEQ ID NO. 1 | 36-49 | | 14 |
| CDR-H2 | AIDPETGGTAYNQKFKD | 50-66 | 4 | 17 |
| HFR3 | Amino acids 67-98 in SEQ ID NO. 1 | 67-98 | | 32 |
| CDR-H3 | TGYQFDY | 99-105 | 5 | 7 |
| HFR4 | Amino acids 67-98 in SEQ ID NO. 1 | 106-116 | | 11 |

MAb035-L
Amino Acid Sequence: (107aa)
SEQ ID NO. 6
DIVMTQSQKFMSTSVGDRVSVTCKASQNVGSNVAWYQQKPGQSPKALIY
SASYRYSGVPDRFAGSGSGTDFTLTISNVQSEDLADYFCQQYNSHPYTF
GGGTKLEIK
>3626-mAb035-13-68A8G10-L (321 bp)
SEQ ID NO. 7
GACATTGTGATGACCCAGTCTCAAAATTCATGTCCACATCAGTAGGAGAC
AGGGTCAAGGCAAGGGTCAAGGCAAGGCCAGTAGTAGTAGTAGTAGTAGT
AGTAGTAGTAGATCTCCTAAGATTGATTTCGGATCTGGATCTGGATCTGG
ACAGATTGATTGTCAGTGATTGTCAGATTGTCAGATTGTCAGATTGTCAG
ATTGTCAGATTGTCAGATTGTCAGATTGTCAGATTGTCAGATTGTCAGAT
GTCAGATGTCAGATGTCAGATGTCAGATGTCAGATGTCAGATGTCAGATG
TCAGATGTCAGATGTCAGATGTCAGATGTCAGATGTCAGATGTCAGATGT
CAGCAATACAACAGCCATCCGTACACGTTCGGAGGGGGACCAACTGGAAA
TAAAA

| | | | Sequence | |
|---|---|---|---|---|
| | | Position in | Number | |
| region | Sequence fragment | SEQ ID NO. 6 | SEQ ID NO. | Length |
| LFR1 | Amino acids 1-23 in SEQ ID NO. 6 | 1-23 | | 23 |
| CDR-L1 | KASQNVGSNVA | 24-34 | 8 | 11 |
| LFR2 | Amino acids 35-49 in SEQ ID NO. 6 | 35-49 | | 15 |
| CDR-L2 | SASYRYS | 50-56 | 9 | 7 |
| LFR3 | Amino acids 57-88 in SEQ ID NO. 6 | 57-88 | | 32 |
| CDR-L3 | QQYNSHPYT | 89-97 | 10 | 9 |
| LFR4 | Amino acids 98-107 in SEQ ID NO. 6 | 98-107 | | 10 |

MAb053-H
Amino Acid Sequence: (112aa)
SEQ ID NO. 11
QVQLKQSGPGLVQPSQSLSIICTVSGFSLSDYGVMWVRQSPGKGLEWL
GVIWSGGSTDYNAAFTSRLSISRDNSKRQVIFKMNSLQAEDTAIHYCV
RETMDYWGQGTSVTVS
>3626-mAb053-22-82G5B7-H (339 bp)
SEQ ID NO. 12
CAGGTGCAGCTGAAGCAGTCAGGACCTGCCTAGTGCAGCCTAGTGCAG
CACAGACCTGTCCTCCCTCACAGACCTGTTCTCTCATTAAGTGACTAT
GGGGTTCTGTAATGTGTGTGTGGATTGGGGTGGGTGGGTGGGTGGGGT
GGGGTGGGGTGGGGTGGGGTGGGGTGGGGTGGGGTGGGGTGGGGTGGG
GGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGCAG
CCATACATTACTGTGTCAGAGAAACGATGGGACTACTGGGGTCAAGGA
ACCTCAGTCGTCTCCTCA

| | | | Sequence | |
|---|---|---|---|---|
| | | Position | Number | |
| | | in SEQ ID | SEQ ID | |
| region | Sequence fragment | NO. 11 | NO. | Length |
| HFR1 | Amino acids 1-30 in | 1-30 | | 30 |
| | SEQ ID NO. 11 | | | |
| CDR-H1 | DYGVM | 31-35 | 13 | 5 |
| HFR2 | Amino acids 36-49 in | 36-49 | | 14 |
| | SEQ ID NO. 11 | | | |
| CDR-H2 | VIWSGGSTDYNAAFTS | 50-65 | 14 | 16 |
| HFR3 | Amino acids 66-97 in | 66-97 | | 32 |
| | SEQ ID NO. 11 | | | |
| CDR-H3 | ETMDY | 98-102 | 15 | 5 |
| HFR4 | Amino acids 103-112 | 103-112 | | 10 |
| | in SEQ ID NO. 11 | | | |

MAb053-L
Amino Acid Sequence: (107aa)
SEQ ID NO. 16
DIQMTQTTSSLSASLGDRVTISCRASQDISNYLNWYQQRPDGTVKRLI
YYTSRLHSGVPSRFSGSGSGTDYSLTISSLEQEDIATYFCQQAHTLPW
TFGGGTKLEIK
>3626-mAb053-22-82G5B7-L (321 bp)
SEQ ID NO. 17
GATATCCAGATGACACAGACTACATCCCCTGTCTGCCTCTCTGGGAGA
CAGAGTCACCATCAGTTGCAGGCAAGTGCAAGTGCAAGTGCAAGATTG
TAAACTGGTAACTGGTAACTGGTAACTGGTAACTGGTAACTGGTAGGG
GTGTGGCAGTGGGTCTGGAACATTAGGGGGTGTGGCACACTGGGGTGT
GGCACACTGGGGGGGGGGGGGGGGGGGGGGGGGGGGGGGGGGGGGGGG
GGGGGGGGGGGGGGGGGGGGGGGGGGGGGGGGGGGGGGGGGGGGGGGG
GGGGGGGGGGGGGGGCTCATACGCTTCCGTGGACGTTCGGGGAGGCAC
CAAGTTGGAGATCAAA

| | | | Sequence | |
|---|---|---|---|---|
| | | Position | Number | |
| | | in SEQ ID | SEQ ID | |
| region | Sequence fragment | NO. 16 | NO. | Length |
| LFR1 | Amino acids 1-23 in | 1-23 | | 23 |
| | SEQ ID NO. 16 | | | |
| CDR-L1 | RASQDISNYLN | 24-34 | 18 | 11 |
| LFR2 | Amino acids 35-49 | 35-49 | | 15 |
| | in SEQ ID NO. 16 | | | |
| CDR-L2 | YTSRLHS | 50-56 | 19 | 7 |
| LFR3 | Amino acids 57-88 in | 57-88 | | 32 |
| | SEQ ID NO. 16 | | | |
| CDR-L3 | QQAHTLPWT | 89-97 | 20 | 9 |
| LFR4 | Amino acids 98-107 | 98-107 | | 10 |
| | in SEQ ID NO. 16 | | | |

MAb041-H
Amino Acid Sequence: (112aa)
SEQ ID NO. 21
EVQLVESGGGLVKPGGSLKLSCAVSGFTFSNSAMSWVRQTPEKRLEWV
ATITDGGDYTYYSDNVKGRFTISRDNAKNNLYLQMSHLKSEDTAMYYC
VRGISYWGQGTLVTVS
>3626-mAb041-17-45E5G11-H (339 bp)
SEQ ID NO. 22
GAAGTGCAGCTGGTGGAGTCTGGGAGGTAGTGAAGCCTGGAGGGCCTG
GAGGGCCTGGAGGGCCTGCAGTCTCTGGATTCTCTGGATTCTGGATTC
TGCCATGGACTCTGCCATGGGGGTTCTGCCATGGAAGAGGCTGCCAGA
CTCTGCCATGGGGGGTTCGCCAGACTCTGCCATGGGGGGTTCGCCAGA
CTCTGCCGGGGGGTTCGCCAGACTCTGCCGGGGGTGGGGTGGGGTCTG
CCAGACTCTGCCGGGGGTGGGGGGGGGGGGGGGGGGGGGGGGGGGGGG
GGGGGGGGGGGGGGGGGGGGGGAGGACACAGCCATGTATTACTGTGTA
AGAGGGATTTCTTACTGGGGCCAAGGACTCTGGTCTGGCCAAGGACTC
TGGTCTCTGCA

| | | | Sequence | |
|---|---|---|---|---|
| | | Position | Number | |
| | | in SEQ ID | SEQ ID | |
| region | Sequence fragment | NO. 21 | NO. | Length |
| HFR1 | Amino acids 1-30 in | 1-30 | | 30 |
| | SEQ ID NO. 21 | | | |
| CDR-H1 | NSAMS | 31-35 | 23 | 5 |
| HFR2 | Amino acids 36-49 | 36-49 | | 14 |
| | in SEQ ID NO. 21 | | | |
| CDR-H2 | TITDGGDYTYYSDNVKG | 50-66 | 24 | 17 |
| HFR3 | Amino acids 67-98 in | 67-98 | | 32 |
| | SEQ ID NO. 21 | | | |
| CDR-H3 | GISY | 99-102 | 25 | 4 |
| HFR4 | Amino acids 103-112 | 103-112 | | 10 |
| | in SEQ ID NO. 21 | | | |

MAb041-L
Amino Acid Sequence: (111aa)
SEQ ID NO. 26
DIVLTQSPASLAVSLGQRATISCRASKSVSTSSDSYMHWYQQKPGQPP
KLLIKYASNLESGVPVRFSGSGSGTDFTLNIHPVEEEDAATYYCQHSR
EFPWTFGGGTKLEIK
>3626-mAb041-17-45E5G11-L (333 bp)
SEQ ID NO. 27
GACATTGTGCTGACACAGTCTCCTGCTTCCTTAGCTGTATCTCTGGGC
AGAGGGCCACAGAGGGCCAGCAGAAAGTGTCAGTACATCTAGCACTGC
ACTGCCCAAACAGGACAGGACAGGACAGGACAGGACAGCCGCCCAAAC
TGCCCAAACTGGGGGGGGGGGGGGGGGGGGGGGGGGGGGGGGGGGGGG
GGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGG
GGGGGGTATTGTCAGCACAGTAGGGAGTTTCCGTGGACGTTCGGGGAG
GCACCAAGCTGGAAATCAAA

| | | | Sequence | |
|---|---|---|---|---|
| | | Position | Number | |
| | | in SEQ ID | SEQ ID | |
| region | Sequence fragment | NO. 26 | NO. | Length |
| LFR1 | Amino acids 1-23 in | 1-23 | | 23 |
| | SEQ ID NO. 26 | | | |
| CDR-L1 | RASKSVSTSSDSYMH | 24-38 | 28 | 15 |
| LFR2 | Amino acids 39-53 | 39-53 | | 15 |
| | in SEQ ID NO. 26 | | | |
| CDR-L2 | YASNLES | 54-60 | 29 | 7 |
| LFR3 | Amino acids 61-92 in | 61-92 | | 32 |
| | SEQ ID NO. 26 | | | |
| CDR-L3 | QHSREFPWT | 93-101 | 30 | 9 |
| LFR4 | Amino acids 102-111 | 102-111 | | 10 |
| | in SEQ ID NO. 26 | | | |

MAb058-H
Amino Acid Sequence: (116aa)
SEQ ID NO. 31
EVQLQQSGPVLVKPGAAVKMSCKASGYIFTDYYIHWVKLSRGESLEWI
GLINPYNGGTNYNQQFTGKATLTVDKSSSTAYVELNSLTSGSSAVYYC
VRRTGYFDYWGQGTTLTVSS
>3626-mAb058-25-71F5C6-H (348 bp)
SEQ ID NO. 32
GAGGTCCAGCTGCAACAGTCTGGACCTGTAGGGAAGCCTGGGGCTGCA
GTGAAGATGTCCTGTAAGATTATTCACTGGGGTGATACATATACACAC
TGGGGGGTGGAAGAGCCTTGGGGGGGGGGGGGGGGGGGGAGAGGCCTT
GGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGACTCT
GCAGTCTATTATTGTGTACGGAGGACTGGATACTTTGGACTGGACTAC
TGGGGCCAAGGCACCACTCTCTCTCCTCCA

| | | | Sequence | |
|---|---|---|---|---|
| | | Position | Number | |
| | | in SEQ ID | SEQ ID | |
| region | Sequence fragment | NO. 31 | NO. | Length |
| HFR1 | Amino acids 1-30 in | 1-30 | | 30 |
| | SEQ ID NO. 31 | | | |
| CDR-H1 | DYYIH | 31-35 | 33 | 5 |
| HFR2 | Amino acids 36-49 | 36-49 | | 14 |
| | in SEQ ID NO. 31 | | | |
| CDR-H2 | LINPYNGGTNYNQQFTG | 50-66 | 34 | 17 |
| HFR3 | Amino acids 67-98 in | 67-98 | | 32 |
| | SEQ ID NO. 31 | | | |
| CDR-H3 | RTGYFDY | 99-105 | 35 | 7 |
| HFR4 | Amino acids 106-116 | 106-116 | | 11 |
| | in SEQ ID NO. 31 | | | |

MAb058-L
Amino Acid Sequence: (112aa)
SEQ ID NO. 36
DVVMTQTPLSLPVSLGDQASISCRSSQSLVHSNGNTYLHWYLQKPGQS
PKLLIYKVSNRFSGVPDRFSGSGSGTDFTLKISRVEAEDLGVYFCSQT
THVPYTFGGGTKLEIK
>3626-mAb058-25-71F5C6-L (336 bp)
SEQ ID NO. 37
GATGTTGGATGACCCAAACTCCACACACTCTCTCCTGCCTGTCAGTCT
TGGAGATCAAGCCTTGCAGATCTAGTCAGAGTCCTTGTAGACACAGTA
GATGGTAGTGTAGATTGTAGTGTAGTGTAGTGTAGTGTAGTGCCGACA
GTGTGGCAGTGATGGGACAGATTTTCGGGGGGGGGGGGGGGGGGGGGG
GGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGG
GGGGGGGGGGTTCTGCTCTCAAACTACACATGTTCCGTACACGTTCGG
AGGGGACCAAGCTGGAAATAAAA

| | | | Sequence | |
|---|---|---|---|---|
| | | Position | Number | |
| | | in SEQ ID | SEQ ID | |
| region | Sequence fragment | NO. 36 | NO. | Length |
| LFR1 | Amino acids 1-23 in | 1-23 | | 23 |
| | SEQ ID NO. 36 | | | |
| CDR-L1 | RSSQSLVHSNGNTYLH | 24-39 | 38 | 16 |
| LFR2 | Amino acids 40-54 | 40-54 | | 15 |
| | in SEQ ID NO. 36 | | | |
| CDR-L2 | KVSNRFS | 55-61 | 39 | 7 |
| LFR3 | Amino acids 62-93 in | 62-93 | | 32 |
| | SEQ ID NO. 36 | | | |
| CDR-L3 | SQTTHVPYT | 94-102 | 40 | 9 |
| LFR4 | Amino acids 103-112 | 103-112 | | 10 |
| | in SEQ ID NO. 36 | | | |

MAb034-H
Amino Acid Sequence (119aa)
SEQ ID NO. 41
EVQLQQSGPVLVKPGASVKMSCKASGYTFTDFYMNWMRQSHGKSLEWI
GVIDPYNGNTSYNQKFKGKATLTVDKSSSTAYMELNSLTSEDSAVYYC
ARGNYYYYAMDYWGQGTSVTVSS
>3626-mAb034-12-68E3A9-H (357 bp)
SEQ ID NO. 42
GAGGTCCAGCTGCAACAGTCTGGACCTGGCTTGGGAAGCCTGGGCTTC
AGTGAAGATGTCCTGTAAGGCTTCTGGAAGATGCCTGTAAGTCTGGAT
GATGGAACTGGATGGAGTGGATGGAGTGGAGTGATGGAGTGCCTTACA
ACGGAGTGATTGGGAGTGATTGGAGTGATTGCCTTACAACGGAGTGAT
TGCCTTAGAGTGATTGCCTTAGAGTGATTGCCTTAGAGTGATTGCCTT
AGAGTGATTGCCTTAGAGTGATTGCCTTAGAGGGGGGGGGGGGGGGGG
GGGGGGGGGGGGGGGGGGGGGTCTGCAGTCTATTACTGTGCAAGAGGT
AACTATTATTACTGGTAACTATTATTACTATGCTATGGACTACTGGGG
GTCAAGGAACTTCAGTCACGGTCTCTCCTCA

| | | | Sequence | |
|---|---|---|---|---|
| | | Position | Number | |
| | | in SEQ ID | SEQ ID | |
| region | Sequence fragment | NO. 41 | NO. | Length |
| HFR1 | Amino acids 1-30 in | 1-30 | | 30 |
| | SEQ ID NO. 41 | | | |
| CDR-H1 | DFYMN | 31-35 | 43 | 5 |
| HFR2 | Amino acids 36-49 | 36-49 | | 14 |
| | in SEQ ID NO. 41 | | | |
| CDR-H2 | VIDPYNGNTSYNQKFKG | 50-66 | 44 | 17 |
| HFR3 | Amino acids 67-98 in | 67-98 | | 32 |
| | SEQ ID NO. 41 | | | |
| CDR-H3 | GNYYYYAMDY | 99-108 | 45 | 10 |
| HFR4 | Amino acids 109-119 | 109-119 | | 11 |
| | in SEQ ID NO. 41 | | | |

MAb034-L
Amino Acid Sequence (111aa)
SEQ ID NO. 46
DIVLTQSPASLAVSLGQRATISCRASESVSIHGTHLMYWYQQKPGQPP
KLLIYAASNLESGVPARFSGSGSETDFTLNIHPVEEEDAATFFCQQSI
EDPPTFGGGTKLEIK
>3626-mAb034-12-68E3A9-L (333 bp)
SEQ ID NO. 47
GACATTGGCTGACCCAATCTCCAGCTTCTTTGGCTGTCTCTAGGGCAG
AGGGCCAGAGGGCCACTCATTCCTGCAGGAAAGTGTCAGTATTCATGG
TAGTGTAGTATGTAGTGTAGTGTAGTAGATGTAGTAGTAGTAGTAGTA
GTAGTAGTAGATGATGGGAGTGGCATTGGCATTGGCATTGGCATTGGC
ATTGGCATTGGCATTGGGTGTGGTGTGGTGGTGGTGGGGGGGGGGGGG
GGGGGGGGGGGGGGGGGGGGGGGGGGGGGGGGGGGGGGGGGGGGGGGG
GGGGGGGGGGGGGGGGGGGTTCTGTCAGCAAAGTATTGAGGATCCTCC
CACGTTCGGAGGGGGACCAAGCTGGAAATAAA

| | | | Sequence | |
|---|---|---|---|---|
| | | Position in | Number | |
| region | Sequence fragment | SEQ ID NO. 46 | SEQ ID NO. | Length |
| LFR1 | Amino acids 1-23 in SEQ ID NO. 46 | 1-23 | | 23 |
| CDR-L1 | RASESVSIHGTHLMY | 24-38 | 48 | 15 |
| LFR2 | Amino acids 39-53 in SEQ ID NO. 46 | 39-53 | | 15 |
| CDR-L2 | AASNLES | 54-60 | 49 | 7 |
| LFR3 | Amino acids 61-92 in SEQ ID NO. 46 | 61-92 | | 32 |
| CDR-L3 | QQSIEDPPT | 93-101 | 50 | 9 |
| LFR4 | Amino acids 102-111 in SEQ ID NO. 46 | 102-111 | | 10 |

MAb049-H
Amino Acid Sequence (117aa)
SEQ ID NO. 51
EVQLQQSGPELVKPGASVKISCKASGHTLTDYYMNWVKQSHGKSPEWIGD
INPNNDGIDYNQKFRGKAILTVDKSSSSFYMELRSLTSEDSAVYYCVGFV
RGYFVSWGQGTTLTVSS
>3626-mAb049-20-80C8B1-H (351 bp)
SEQ ID NO. 52
GAGGTCCAGCTGCAACAATCTGGACCTGAGCTGGTGAAGCCTGGGCTTCA
GTGAAGATTTCCTGTAAGGCTTCTGTAAGATTGGGGGAAGAGCCATGGGG
ATTGGATTGGATTGGATTGGAAGATTAGCCTAATAGATTGGATTGGATTG
GATTGGATTGGATTGGATTGATTGCCATATTGACATTGACATTGACATTG
GAACTGAGGGGGGGACATTGACATTGACATTGACATGGGGGGGGGGGGGG
GGGGGGGGGGGGGGGGGGGGGGGGGGGGGGGGGGGGGGGGGGGGGGGGGG
GGGGGGGGGGTCTGCAGTCTATTATTGTGGGGGTTCGTACGAGGCTACTT
TTGTCTCCTGGGCCAAGCACCACTCTCTCCTCA

| | | | Sequence | |
|---|---|---|---|---|
| | | Position in | Number | |
| region | Sequence fragment | SEQ ID NO. 51 | SEQ ID NO. | Length |
| HFR1 | Amino acids 1-30 in SEQ ID NO. 51 | 1-30 | | 30 |
| CDR-H1 | DYYMN | 31-35 | 53 | 5 |
| HFR2 | Amino acids 36-49 in SEQ ID NO. 51 | 36-49 | | 14 |
| CDR-H2 | DINPNNDGIDYNQKFRG | 50-66 | 54 | 17 |
| HFR3 | Amino acids 67-98 in SEQ ID NO. 51 | 67-98 | | 32 |
| CDR-H3 | FVRGYFVS | 99-106 | 55 | 8 |
| HFR4 | Amino acids 107-117 in SEQ ID NO. 51 | 107-117 | | 11 |

MAb049-L
Amino Acid Sequence (106aa)
SEQ ID NO. 56
DIQMTQTTSSLSASLGDRVTINCRASQDISNYLNWYQQKPDGTVKLLIYY
TSRLRSGVPSRFSGSGSGTDYSLTINNLDQEDFATYFCQQGNTLPWTFGG
GTKLEI
>3626-mAb049-20-80C8B1-L (321 bp)
SEQ ID NO. 57
GATATCCAGATGACACAGACTACATCCCCCTGTCTGCCTCTCTGGGAGAC
AGAGTCACATTGCAGGCAAGTGCAAGTAATTGGTATTGTATCAGCAGAAA
CCAGATGGGAACTGGTATTGTAAACTGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGTAATACGCT
TCCGTGGACGTTCGGTGGAGGCACCAAGCTGGAAATCAAA

| | | | Sequence | |
|---|---|---|---|---|
| | | Position in | Number | |
| region | Sequence fragment | SEQ ID NO. 56 | SEQ ID NO. | Length |
| LFR1 | Amino acids 1-23 in SEQ ID NO. 56 | 1-23 | | 23 |
| CDR-L1 | RASQDISNYLN | 24-34 | 58 | 11 |
| LFR2 | Amino acids 35-49 in SEQ ID NO. 56 | 35-49 | | 15 |
| CDR-L2 | YTSRLRS | 50-56 | 59 | 7 |
| LFR3 | Amino acids 57-88 in SEQ ID NO. 56 | 57-88 | | 32 |
| CDR-L3 | QQGNTLPWT | 89-97 | 60 | 9 |
| LFR4 | Amino acids 98-106 in SEQ ID NO. 56 | 98-106 | | 9 |

MAb064-H
Amino Acid Sequence (116aa)
SEQ ID NO. 61
QVQLKESGPGLVAPSQTLSITCSVSGFSLTNYAVSWVRQPPGKGLEWLGV
MWGDGNTNYHSTLISRLSISKDNSKSQVFLKLNSLQTDDTATYYCARSSG
WYFDVWGTGTTVTVSS
>3626-mAb064-27-80E5G2-H (348 bp)
SEQ ID NO. 62
CAGGTGCAGCTGAAGGAGTCAGGACCTGCCTGGGCCTGGGCCCTCACAGA
CCCCTGTTCTCAGGTTCTCTCTAACCAACTATGCTGTAAAGGGGGGTTCG
CCAGGTGTTCGCCAGGAAGGTGTGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGCACAGCCACGTACCTGTG
CCCGTAGTAGCGCTGGTAGTAGCGCTGGTACTTCGATGTCTGGGGCACAG
GACCACGGGCACCCGTCTCCTCTCTCCTCA

| | | | Sequence | |
|---|---|---|---|---|
| | | Position in | Number | |
| region | Sequence fragment | SEQ ID NO. 61 | SEQ ID NO. | Length |
| HFR1 | Amino acids 1-30 in SEQ ID NO. 61 | 1-30 | | 30 |
| CDR-H1 | NYAVS | 31-35 | 63 | 5 |
| HFR2 | Amino acids 36-49 in SEQ ID NO. 61 | 36-49 | | 14 |
| CDR-H2 | VMWGDGNTNYHSTLIS | 50-65 | 64 | 16 |
| HFR3 | Amino acids 66-97 in SEQ ID NO. 61 | 66-97 | | 32 |
| CDR-H3 | SSGWYFDV | 98-105 | 65 | 8 |
| HFR4 | Amino acids 106-116 in SEQ ID NO. 61 | 106-116 | | 11 |

MA6064-L
Amino Acid Sequence (107aa)
SEQ ID NO. 66
DIQMTQTTPSLSVTLGDRVTIRCRASQDINNYLNWYQQKPDGTVKLLIYY
TSESRSGVPSRFSGSGSGTDYSLTISNLEQEDIATYFCQQGYALPPAFGG
GTKLEIK
>3626-mAb064-27-80E5G2-L (321 bp)
SEQ ID NO. 67
GATATCCAGATGACACACAGACTACACCTCCCTTGACTCTGGACTCTGGG
AGACAGAGTCACTCTGGGCAAGTGCAAGTGCAAGTGCAAGTGGACATTAA
CAATTATTTAAATTGGTATCAGAGAAGATTGGTATCAGAAGATGGGAACT
GGGAACTGGGAACTGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
TTATGCGCTTCCTCCGGCGTTCGGTGGAGGCACCAAGCTGGAAATAAAA

| | | | Sequence | |
|---|---|---|---|---|
| | | Position in | Number | |
| Region | Sequence fragment | SEQ ID NO. 66 | SEQ ID NO. | Length |
| LFR1 | Amino acids 1-23 in SEQ ID NO. 66 | 1-23 | | 23 |
| CDR-L1 | RASQDINNYLN | 24-34 | 68 | 11 |
| LFR2 | Amino acids 35-49 in SEQ ID NO. 66 | 35-49 | | 15 |
| CDR-L2 | YTSESRS | 50-56 | 69 | 7 |
| LFR3 | Amino acids 57-88 in SEQ ID NO. 66 | 57-88 | | 32 |
| CDR-L3 | QQGYALPPA | 89-97 | 70 | 9 |
| LFR4 | Amino acids 98-107 in SEQ ID NO. 66 | 98-107 | | 10 |

MAb033-H
Amino Acid Sequence: (118aa)
SEQ ID NO. 71
QVQLQQSGAELVRPGTAVRVSCKASGYAFTNYFIEWVKQRPGQGLEWIGV
INPGSGDTYYSEKFQGKATLTADKSSSTAYMQLSSLTSEDSAVYFCARED
DYSWFTYWGQGTLVTVSA
>3626-mAb033-11-67G9A3-H (354 bp)
SEQ ID NO. 72
CAGGTCCAGCTGCAGCAGTCTGGAGCTGAGTTGTAAGGCCTGGACTGCAG
TGGATGGATACGCCTTCACTAATTATATATATATATATATATATAGTGGA
TTGGATTGGATTGGATTAGTGATTAGTGATTAGATTAGTCCTGGATTGAT
TAGATTAGTCCTGCAAGGACTGACTGACTGACTGCCTAGGACAGTCCTAG
GACTGACTGCCTAGCACTGACTGCCTAGCACTGCCTAGCACTGCCTAGCA
CTGCCTAGCACTGCCTAGCACTGCCTAGCACTGCCTAGCACTGCCTAGCA
CTGCCTACATGCCTAGTGACTGACTGACTGACTGACTGACTGACTGACTG
ACTGACTGAGGACTCTGCGGTCTATTTCTGTGCAAGAGAGGATGATTACT
CCTGGTTTACTTACTGGGCCAAGGACTCTGGTCTCTGTCTCTGCA

| | | | Sequence | |
|---|---|---|---|---|
| | | Position in | Number | |
| Region | Sequence fragment | SEQ ID NO. 111 | SEQ ID NO. | Length |
| HFR1 | Amino acids 1-30 in SEQ ID NO. 111 | 1-30 | | 30 |
| CDR-H1 | NYFIE | 31-35 | 73 | 5 |
| HFR2 | Amino acids 36-49 in SEQ ID NO. 111 | 36-49 | | 14 |
| CDR-H2 | VINPGSGDTYYSEKFQG | 50-66 | 74 | 17 |
| HFR3 | Amino acids 67-98 in SEQ ID NO. 111 | 67-98 | | 32 |
| CDR-H3 | EDDYSWFTY | 99-107 | 75 | 9 |
| HFR4 | Amino acids 108-118 in SEQ ID NO. 111 | 108-118 | | 11 |

MAb033-L
Amino Acid Sequence: (111aa)
SEQ ID NO. 76
DIVLTQSPASLAVSLGQRATISCRASENVDNYGISFMHWYRQKPGQPPKL
LIYRASNLESGIPARFSGSGSRTDFTLTINPVETDDVATYYCQQSNKDPY
TFGGGTKLEIK
>3626-mAb033-11-67G9A3-L (333 bp)
SEQ ID NO. 77
GACATTGGCTGACCCAATCTCCAGCTTCTTTGGCTGTGTCTCTAGGGCAG
AGGCCAGAGGGCAGAGATTGGCATTAGTTTATGGCATTAGTTTATGGCAT
TAGTTTATGGCATTAGTTTATGGCACTGGTAGGCAGAAGTTATGCACTGG
GATCCGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGCTATTACT
GTCAGCAAAGTAATAAGGATCCGTACACGTTCGGAGGGGACCAAACTGGA
AATAAAA

| | | | Sequence | |
|---|---|---|---|---|
| | | Position in | Number | |
| Region | Sequence fragment | SEQ ID NO. 116 | SEQ ID NO. | Length |
| LFR1 | Amino acids 1-23 in SEQ ID NO. 116 | 1-23 | | 23 |
| CDR-L1 | RASENVDNYGISFMH | 24-38 | 78 | 15 |
| LFR2 | Amino acids 39-53 in SEQ ID NO. 116 | 39-53 | | 15 |
| CDR-L2 | RASNLES | 54-60 | 79 | 7 |
| LFR3 | Amino acids 61-92 in SEQ ID NO. 116 | 61-92 | | 32 |
| CDR-L3 | QQSNKDPYT | 93-101 | 80 | 9 |
| LFR4 | Amino acids 102-111 in SEQ ID NO. 116 | 102-111 | | 10 |

MAb047-H
Amino Acid Sequence: (117aa)
SEQ ID NO. 81
EVQLVESGGGLVKPGGSLKLSCAASGFTFSDYGMYWIRQAPEKGLEWVAY
ISNGGSSIYYADTVKGRFTVSRDNAKNTLFLQMTSLRSEDTAIYYCARGG
ELGFAYWGQGTLVTVSA
>3626-mAb047-19-76C9F6-H (351 bp)
SEQ ID NO. 82
GAGGTGCAGCTGGTGGAGTCTGGGAGGTAGTGAAGCCTGGAGGCCTGGAG
GGCCTGGAGGGCCTGGAGACTCTCCTGGATTCTTGATTCTGATTCTGATT
CTGATTGACTGGATTGGATGGACTGGATTGGATTGGATGGATGGGGGGAT
GGATGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGCTGAGGACACGGCCA
TATTACTGTGCAAGGGGAGAGAACTCGGGTTTGCTTACTGGGCCAAGGAC
TCTGGTCTCTGGTCTCTGCA

| | | | Sequence | |
|---|---|---|---|---|
| | | Position in | Number | |
| Region | Sequence fragment | SEQ ID NO. 121 | SEQ ID NO. | Length |
| HFR1 | Amino acids 1-30 in SEQ ID NO. 121 | 1-30 | | 30 |
| CDR-H1 | DYGMY | 31-35 | 83 | 5 |
| HFR2 | Amino acids 36-49 in SEQ ID NO. 121 | 36-49 | | 14 |
| CDR-H2 | YISNGGSSIYYADTVKG | 50-66 | 84 | 17 |
| HFR3 | Amino acids 67-98 in SEQ ID NO. 121 | 67-98 | | 32 |
| CDR-H3 | GGELGFAY | 99-106 | 85 | 8 |
| HFR4 | Amino acids 107-117 in SEQ ID NO. 121 | 107-117 | | 11 |

MAb047-L
Amino Acid Sequence: (105aa)
SEQ ID NO. 86
DIQMTQSPASLSVSVGETVTITCRASENIYSNLAWYQQKQGKSPQLLVYA
AINLVEGVPSRFSGSGSGTQYSLKINSLQSEDFGSYFCQHFWGLPYTFGG
GTKLE
>3626-mAb047-19-76C9F6-L (321 bp)
SEQ ID NO. 87
GACATTCAGATGACTCAGTCTCCAGCCCCCTATCTGTATCTGTATCTGGG
AGAAACTGTCACCATGTGTCGAGAAAACTGTCAAAGTGTGGAAAGTGGTA
TCAGTAGAAAATTGGGTATCAGTAGATGGGAAAATCTCCTCAGTGGCATC
AGATGGTGTGGCATCAGATGGATGTGGCATCAGATGGATGTGGCATCAGA
TGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGACATTTTTGGGTCTTCCGTACACGTTCGGAGGGGACCAAGCTGG
AAAAT

| | | | Sequence | |
|---|---|---|---|---|
| | | Position in | Number | |
| Region | Sequence fragment | SEQ ID NO. 126 | SEQ ID NO. | Length |
| LFR1 | Amino acids 1-23 in SEQ ID NO. 126 | 1-23 | | 23 |
| CDR-L1 | RASENIYSNLA | 24-34 | 88 | 11 |
| LFR2 | Amino acids 35-49 in SEQ ID NO. 126 | 35-49 | | 15 |
| CDR-L2 | AAINLVE | 50-56 | 89 | 7 |
| LFR3 | Amino acids 57-88 in SEQ ID NO. 126 | 57-88 | | 32 |
| CDR-L3 | QHFWGLPYT | 89-97 | 90 | 9 |
| LFR4 | Amino acids 98-105 in SEQ ID NO. 126 | 98-105 | | 8 |

MAb055-H
Amino Acid Sequence: (112aa)
SEQ ID NO. 91
QVQLKQSGPGLLQPSQSLSITCTVSGFSLDEYGVHWFRQSPGKGLEWLGV
IWSGGSTDYNAAFISRLSIRKDNSKSQIFFNMNSLQADDTAIYYCVREEW
EYWGQGTTLTVS
>3626-mAb055-24-83D3H2-H (339 bp)
SEQ ID NO. 92
CAGGTGCAGCTGAAGCAGTCAGGACCTGCCTACTGCAGCCTACTGCACAG
AGCCTGTCCTTCACGTCTCTGCACAGTCTCTGTTAGATGAGTATGGTTTC
TGTAGATGAGTGTGTGTGTGTGTGTGTGTGGAGTGGAGTGGAGTGGAGTG
GAGTGGAGTGGAGTGGAGTGGAGTGGAGTGGAGTGGAGTGGAGTGGGAGT
GGAGTGGAGTGGAGTGCACAGACAGGACAATTCCAAGAGACAATTCCAAA
TTTCTTTAATATGACAGATGATGATGATGATGATGATGATGATGATGATG
ATGATGATGATGATCACAGCCATATTACTGTGTCAGAGAGGAATGGGAAT
ATTGGGGCCAAGGCACACTCTCCACAGTCTCCTCA

| | | | Sequence | |
|---|---|---|---|---|
| | | Position in | Number | |
| Region | Sequence fragment | SEQ ID NO. 141 | SEQ ID NO. | Length |
| HFR1 | Amino acids 1-30 in SEQ ID NO. 141 | 1-30 | | 30 |
| CDR-H1 | EYGVH | 31-35 | 93 | 5 |
| HFR2 | Amino acids 36-49 in SEQ ID NO. 141 | 36-49 | | 14 |
| CDR-H2 | VIWSGGSTDYNAAFIS | 50-65 | 94 | 16 |
| HFR3 | Amino acids 66-97 in SEQ ID NO. 141 | 66-97 | | 32 |
| CDR-H3 | EEWEY | 98-102 | 95 | 5 |
| HFR4 | Amino acids 103-112 in SEQ ID NO. 141 | 103-112 | | 10 |

MA11055-L
Amino Acid Sequence: (107aa)
SEQ ID NO. 96
DIQMTQTTSSLSASLGDRVTISCRPSQDIDNYLNWFQQRPDGTIKLLIYY
TSRLHSGVPSRFSGSGSGTDYSLTINNLEQEDIATYFCQQAHTLPYTFGG
GTKLEIK
DNA sequence:
SEQ ID NO. 97
GATATCCAGATGACACAGACTACATCCCCCTGTCTGCCTCTCTGGGAGAC
AGAGTCACAGTTGCAGCCAAGTTGCAGGACAGTATTTAAACTTGGTTTCA
GCAGAGAGAAACTGGTTGTTTCAGCAGAGAGACAGATTCCTGGCATGATG
ATGATGGGTGTGGCATGATGGGTGTGTGTGTGTGTGTGTGTGTGATGTGT
GTGATGTGTGTGATGTGTGTGATGTGTGTGGCACTGATTGTGTGATGTGT
GATGTGTGATGTGTGATGTGTGATGTGTGTGATGTGTGTGTGTGTGTGTG
TGTGTGTGCTCATACGCTTCCGTACACGTTCGGAGGGGGGACCAACTGGA
AATAAAA

| | | | Sequence | |
|---|---|---|---|---|
| | | Position in | Number | |
| Region | Sequence fragment | SEQ ID NO. 146 | SEQ ID NO. | Length |
| LFR1 | Amino acids 1-23 in SEQ ID NO. 146 | 1-23 | | 23 |
| CDR-L1 | RPSQDIDNYLN | 24-34 | 98 | 11 |
| LFR2 | Amino acids 35-49 in SEQ ID NO. 146 | 35-49 | | 15 |
| CDR-L2 | | 50-56 | 99 | 7 |
| LFR3 | Amino acids 57-88 in SEQ ID NO. 146 | 57-88 | | 32 |
| CDR-L3 | | 89-97 | 100 | 9 |
| LFR4 | Amino acids 98-107 in SEQ ID NO. 146 | 98-107 | | 10 |

Humab035-24-VH
Amino Acid Sequence (116aa)
SEQ ID NO. 101
QVQLVQSGAEVKKPGASVKVSCKASDYTFTDYEMHWVRQAPGQGLEWIGA
IDPETGGTAYNQKFKDRVTMTADTSTSTVYMELSSLRSEDTAVYYCAKTG
YQFDYWGQGTTVTVSS
DNA sequence (348 bp)
SEQ ID NO. 102
CAGGTGCAGCTGGTGCAGTCAGGAGAGAGAGAAGCCAGGCGCAAGCGGAA
GGTCTTGCAAGGCCAGCGACTAGAGATGCATTGCCGGGACTAGAGATGCA
TTGGGGATTGGGATTGGGATTGCACGGGATTGGGATTGACCGAGGAGAGA
GAGAGAGAGAGAGAGAGAGAGAGAGAGAGAGAGAGAGAGAGAGAGAGAGA
GAGAGAGAGAGAGAGAGAGAGAGAGAGAGAGAGAGAGAGAGAGAGAGAGA
GAGAGAGAGAGAGAGAGAGAGAGAGAGAGAGCGAGGACACCGCCGTGTAC
TATTGCGCCAAGACCGGCTACCAGGGCTACCAGTTCGACTATTGGGGGCC
AGGAACCAGTGACAGTGTCCAGC
Humab035-24-VL
Amino Acid Sequence (107aa)
SEQ ID NO. 103
DIQMTQSPSSVSASVGDRVTITCKASQNVGSNVAWYQQKPGKAPKALIYS
ASYRYSGVPSRFSGSGSGTDFTLTISSLQPEDFATYYCQQYNSHPYTFGQ
GTKLEIK
DNA sequence (321 bp)
SEQ ID NO. 104
GACATTCACAGATGACCCAGACCCTAGACCCTAGCAGCGTGCCGCTAGCG
GGGAGAGTGACCGCGCGGGAGAGTGCCAGCCAGGGCTTGGGCAGCAGGGC
TTGGTATCAGCAGAGGCCTAGGGAGGCCTAGGGAGGCCTAGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGGGGGGGGGGGGGGGGG
GGGGGGGGGGGGGGGGGGGGGGGGGGGGGGGGGGTACAACAGCCACCCCC
TACACTTCGGACAGGGCACAGCTGGAGATCAAG

1. A heavy chain variable region of an antibody having a complementarity determining region CDR selected from the group consisting of:
a VH-CDR1 as shown in SEQ ID NO. 10n+3,
a VH-CDR2 as shown in SEQ ID NO. 10n+4, and
a VH-CDR3 as shown in SEQ ID NO. 10n+5;
wherein each n is independently 0, 1, 2, 3, 4, 5, 6, 7, 8 or 9;
wherein any one of the above amino acid sequences further includes a derivative sequence that is optionally added, deleted, modified, and/or substituted for at least one amino acid and can retain the binding affinity of OX40.

2-4. (canceled)

5. An antibody having:
(1) a heavy chain variable region of claim 1; and/or
(2) a light chain variable region having a complementarity determining region CDR selected from the group consisting of:
a VL-CDR1 as shown in SEQ ID NO. 10n+8,
a VL-CDR2 as shown in SEQ ID NO. 10n+9, and
a VL-CDR3 as shown in SEQ ID NO. 10n+10;
wherein each n is independently 0, 1, 2, 3, 4, 5, 6, 7, 8 or 9;
wherein any one of the above amino acid sequences further includes a derivative sequence that is optionally added, deleted, modified, and/or substituted for at least one amino acid and can retain the binding affinity of OX40.

6. The antibody of claim 5, wherein the antibody has a heavy chain variable region and a light chain variable region of claim 3;
wherein the heavy chain variable region and the light chain variable region comprise a CDR selected from the group consisting of:

| VH-CDR1 | VH-CDR2 | VH-CDR3 | VL-CDR1 | VL-CDR2 | VL-CDR3 |
|---|---|---|---|---|---|
| Sequence | Sequence | Sequence | Sequence | Sequence | Sequence |
| Number | Number | Number | Number | Number | Number |
| 3 | 4 | 5 | 8 | 9 | 10 |
| 13 | 14 | 15 | 18 | 19 | 20 |
| 23 | 24 | 25 | 28 | 29 | 30 |
| 33 | 34 | 35 | 38 | 39 | 40 |
| 43 | 44 | 45 | 48 | 49 | 50 |
| 53 | 54 | 55 | 58 | 59 | 60 |
| 63 | 64 | 65 | 68 | 69 | 70 |
| 73 | 74 | 75 | 78 | 79 | 80 |
| 83 | 84 | 85 | 88 | 89 | 90 |
| 93 | 94 | 95 | 98 | 99 | 100 |

wherein any one of the above amino acid sequences also includes a derivative sequence that is optionally added, deleted, modified, and/or substituted for at least one amino acid and can retain the binding affinity of OX40.

7. The antibody of claim 5, wherein the heavy chain variable region of the antibody contains the amino acid sequence as shown in SEQ ID NO. 101, 1, 11, or 21; and/or the light chain variable region of the antibody contains the amino acid sequence as shown in SEQ ID NO. 103, 6, 16 or 26.

8. The antibody of claim 6, wherein the antibody is selected from the group consisting of:

| Antibody | | VH sequence | VL Sequence |
|---|---|---|---|
| number | Clone | Number | Number |
| 1 | Humab035-24 | 101 | 103 |
| 2 | Mab035 | 1 | 6 |
| 3 | Mab053 | 11 | 16 |
| 4 | Mab041 | 21 | 26 |
| 5 | Mab058 | 31 | 36 |
| 6 | Mab034 | 41 | 46 |
| 7 | Mab049 | 51 | 56 |
| 8 | Mab064 | 61 | 66 |
| 9 | Mab033 | 71 | 76 |
| 10 | Mab047 | 81 | 86 |
| 11 | Mab055 | 91 | 96. |

9. A recombinant protein comprising:
(i) an antibody of claim 5; and
(ii) an optional tag sequence to assist in expression and/or purification.

10. A polynucleotide encoding a polypeptide of
the recombinant protein of claim 9.

11. The polynucleotide of claim 10, wherein the polynucleotide encoding the variable region of the heavy chain is shown in SEQ ID NO. 102, 2, 12, 22, 32, 42, 52, 62, 72, 82, or 92; and/or, the polynucleotide encoding the variable region of the light chain is shown in SEQ ID NO. 104, 7, 17, 27, 37, 47, 57, 67, 77, 87 or 97.

12. The polynucleotide of claim 11, wherein the polynucleotide encoding the heavy chain variable region sequence and the polynucleotide encoding the light chain variable region sequence are selected from the group consisting of:

| | Sequence numbering | Sequence numbering |
|---|---|---|
| | of polynucleotide | of polynucleotide |
| Clone | encoding VH | encoding VL |
| Humab035-24 | 102 | 104 |
| Mab035 | 2 | 7 |
| Mab053 | 12 | 17 |
| Mab041 | 22 | 27 |
| Mab058 | 32 | 37 |
| Mab034 | 42 | 47 |
| Mab049 | 52 | 57 |
| Mab064 | 62 | 67 |
| Mab033 | 72 | 77 |
| Mab047 | 82 | 87 |
| Mab055 | 92 | 97. |

13. A vector comprising the polynucleotide of claim 10.

14. A genetically engineered host cell comprising the vector of claim 13.

15. An antibody conjugate comprising:
(a) an antibody portion, which is selected from the group consisting of an antibody of claim 5; and
(b) a coupling portion coupled to the antibody portion, the coupling portion is selected from the group consisting of a detectable marker, drug, toxin, cytokine, radionuclide, enzyme, and a combination thereof.

16. An immune cell that expresses or is exposed outside the cell membrane with the antibody of claim 5.

17. A pharmaceutical composition comprising:
(i) an active ingredient, the active ingredient is selected from the group consisting of: the antibody of claim 5, a recombinant protein comprising the antibody, the antibody conjugate comprising the antibody, the immune cell expressing the antibody, and a combination thereof; and
(ii) a pharmaceutically acceptable carrier.

18. A method of preventing and/or treating diseases related to abnormal expression or function of OX40, which comprises: administering the antibody of claim 5, a recombinant protein comprising the antibody, the antibody conjugate comprising the antibody, the immune cell expressing the antibody and a combination thereof to a subject in need of.

19. The method of claim 18, wherein the diseases related to the abnormal expression or function of OX40 are selected from the group consisting of cancer and autoimmune diseases.

20. The method of claim 18, wherein the cancer is selected from the group consisting of: breast cancer, melanoma, B-cell lymphoma, head and neck cancer, colon cancer, prostate cancer, lung cancer.

21. A reagent kit, which comprises:
(1) a first container containing the antibody of claim 5; and/or
(2) a second container containing a second antibody against the antibody of claim 5.

22. A pharmaceutical combination comprising:
(I) a first active ingredient comprising the antibody of claim 5, a recombinant protein comprising the antibody, the antibody conjugate comprising the antibody, the immune cell expressing the antibody, or the pharmaceutical composition comprising the antibody, and a combination thereof;
(ii) a second active ingredient comprising a second antibody, or a chemotherapeutic agent.

23. The pharmaceutical combination of claim 22, wherein the second antibody is selected from the group consisting of: CTLA4 antibody, PD-1 antibody, PD-L1 antibody, 4-1BB antibody.

24. The pharmaceutical combination of claim 22, wherein the chemotherapeutic agent is selected from the group consisting of docetaxel, carboplatin, and a combination thereof.
